(19) United States
(12) Patent Application Publication
(10) Pub. No.: US 2022/0213055 A1
(43) Pub. Date: Jul. 7, 2022

(54) SUBSTITUTED (PIPERIDIN-1-YL)ARYL ANALOGUES FOR MODULATING AVIL ACTIVITY

(71) Applicant: University of Virginia Patent Foundation, Chorlottesville, VA (US)

(72) Inventors: Hui Li, Charlottesville, VA (US); Zhongqiu Xie, Charlottesville, VA (US); Pawel Lukasz Janczyk, Charlottesville, VA (US); Michael Kenneth Hilinski, Charlottesville, VA (US); Philip Lee Hahn, Charlottesville, VA (US); Johnathon Daniel Dooley, Charlottesville, VA (US)

(21) Appl. No.: 17/607,577

(22) PCT Filed: May 2, 2020
(86) PCT No.: PCT/US2020/031212

§ 371 Date: Oct. 29, 2021

Related U.S. Application Data
(60) Provisional application No. 62/842,307, filed on May 2, 2019.

(51) Int. Cl. C07D 401/04; C07D 409/06; A61K 31/44; A61K 31/444; A61K 31/506; A61K 31/53; A61K 31/497; A61K 45/06

(52) U.S. Cl. CPC C07D 401/04; C07D 409/06; A61K 31/44; A61K 45/06; A61K 31/506; A61K 31/53; A61K 31/497; A61K 31/444

(57) ABSTRACT

In one aspect, the disclosure relates to compounds useful to regulate, limit, or inhibit the expression of AVIL (advillin), methods of making same, pharmaceutical compositions comprising same, and methods of treating disorders associated with AVIL dysregulation using same. In aspects, the disclosed compounds, compositions and methods are useful for treating disorders or diseases in which the regulation, limitation, or inhibition of the expression of AVIL can be clinically useful, such as, for example, the treatment of cancer. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 62/842,307, filed on May 2, 2019, which is incorporated herein by reference in its entirety.

### BACKGROUND

Understanding the molecular mechanisms involved genesis and maintenance of cancer, as well as developing therapies which may help prevent or manage cancerous growth continue to be active areas of research. Work over the last decade is providing evidence that at least certain kinds of cancers may depend on a single oncogene or oncogenic pathway for growth, proliferation and survival. Oncogene addiction describes a phenomenon according to which tumor cells become reliant on the activity of a particular oncogene and die once this activity is inhibited. (Vivanco, 2014; Weinstein, 2002; Weinstein and Joe, 2006). Many recent targeted cancer therapies exploit this concept (Lord and Ashworth, 2013; Luo et al., 2009). The challenge is to find such key oncogenes.

Despite advances in cancer research, better treatment options, and identification of novel therapeutic targets are needed as is identification of novel small molecule modulators of such targets. These needs and other needs are satisfied by the present disclosure.

### BRIEF DESCRIPTION OF THE FIGURES

The accompanying figures, which are incorporated in and constitute a part of this specification, illustrate several aspects and together with the description serve to explain the principles of the disclosure.

FIG. 1 is a graph illustrating IC₅₀ data for the compound PLH1252 in GMB cell line U87 vs astrocytes.

FIGS. 2A and 2B show representative data demonstrating disruption of AVIL protein binding to actin by five test compounds: PLH1252, PLH1295, PLH2056, and PLH2069 (DMSO control). 293T cells were first transfected with MYC tagged AVIL. After 24 hrs, the cells were treated with the IC₅₀ dose of the five compounds for 24 hrs. Immunoprecipitation was performed with MYC antibody, and Western blot was performed with β-actin antibody.

FIG. 3 is a graph illustrating down-regulation of LIN28B gene expression by 5 test compounds, PLH1252, PLH1295, PLH2056, and PLH2069, as measured by qRT-PCR

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects of the present disclosure will employ, unless otherwise indicated, techniques of molecular biology, microbiology, organic chemistry, biochemistry, physiology, cell biology, blood vessel biology, and the like, which are within the skill of the art. Such techniques are explained fully in the literature.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “advillin” and “AVIL” can be used interchangeably, and refer to a protein encoded by a gene in humans with a cytogenetic location of 12q14.1 and a molecular location of base pairs 57,797,376 to 57,818,704 on chromosome 12 (Homo sapiens Annotation Release 109, GRCh38.p12). The protein encoded by this gene is a member of the gelsolin/villin family of actin-regulatory proteins. AVIL has also been referred to as Actin-binding protein DOC6, DOC6, P92, ADVIL.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof. Thus, the subject can be a human, non-human primate, horse, pig, rabbit, dog, sheep, goat, cow, cat, guinea pig or a rodent. The term does not denote a particular age or sex. Moreover, adult and newborn subjects, as well as fetuses, whether male or female, are intended to be included. A “patient” refers to a subject afflicted with a clinical condition, disease or disorder.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer, or a disease or disorder associated with increased, aberrant, or dysfunctional levels of AVIL. The effect can be therapeutic in terms of a partial or complete cure of a disease, condition, symptom or adverse effect attributed to the disease, disorder, or condition. The term “treatment” as used herein can include any treatment of cancer, or a disease or disorder associated with increased, aberrant, or dysfunctional levels of AVIL, in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay. For example, an IC₅₀ for the activity of a compound disclosed herein can be determined in an in vitro or cell-based assay system using the methods described herein. Frequently, an assay, including suitable assays for AVIL, can make use of a suitable cell-line, e.g. a cell line that either expresses endogenously a target of interest, or has been transfected with a suitable expression vector that directs expression of a recombinant form of the target such as AVIL. For example, the IC₅₀ for the compounds disclosed herein can be determined using appropriate cells lines, e.g., glioblastoma cells (U87) and astrocyte cells (non-cancer control).

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

The terms “alkoxy” and “alkoxyl” as used herein to refer to an alkyl or cycloalkyl group bonded through an ether linkage; that is, an “alkoxy” group can be defined as -OA¹ where A¹ is alkyl or cycloalkyl as defined above. “Alkoxy” also includes polymers of alkoxy groups as just described; that is, an alkoxy can be a polyether such as -OA¹-OA² or -OA¹-(OA²)ₐ-OA³, where “a” is an integer of from 1 to 200 and A¹, A², and A³ are alkyl and/or cycloalkyl groups.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The terms “halo,” “halogen” or “halide,” as used herein can be used interchangeably and refer to F, Cl, Br, or I.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The term “hydroxyl” or “hydroxy” as used herein is represented by the formula —OH.

The term “nitro” as used herein is represented by the formula —NO₂.

The term “nitrile” or “cyano” as used herein is represented by the formula —CN.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

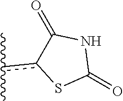

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

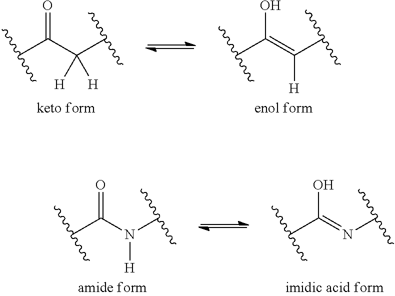 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

It is understood, that unless otherwise specified, temperatures referred to herein are based on atmospheric pressure (i.e. one atmosphere).

In various aspects, the present disclosure pertains to certain compounds and methods that are useful to regulate, limit, or inhibit the expression of AVIL (advillin) in tissue of a mammal, particularly where there are increased, aberrant, or dysfunctional levels of AVIL. It has been found that AVIL expression, or over-expression, is associated with the genesis and growth of certain forms of cancerous tumors. For example, it has been found that AVIL is overexpressed in the vast majority, if not all of human glioblastomas (GBMs). It has been found that GBM cells depend on the overexpression of AVIL for increased survival and migration. Silencing AVIL induced GBM cell death in vitro, and prevented GBM xenograft formation and growth in animal models. Silencing AVIL also dramatically changed cell morphology, and reduced cell migration/invasion ability. In contrast, normal astrocytes express very low levels of AVIL, and silencing AVIL had no obvious effect on cell growth or morphology of normal astrocytes. Clinically, higher expression of AVIL has been correlated with worse patient outcome in GBMs as well as in lower-grade gliomas. In addition to gliomas, lung cancer, bladder cancer, and renal cancer patients with high level of AVIL expression also had worse prognosis. Therefore, in various aspects of the present disclosure, certain compounds are provided that are effective to regulate, limit, or inhibit the expression of AVIL. The present disclosure also provides methods of using the compounds of the present disclosure for targeting AVIL as an oncogene and as a therapeutic target and for treating a disease associated with increased, aberrant, or dysfunctional levels of AVIL, such as cancer. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Compounds.

In various aspects, the present disclosure pertains to certain compounds that are useful to regulate, limit, or inhibit the expression of AVIL (advillin) in cells of a mammal having increased, aberrant, or dysfunctional levels of AVIL, which will have use as therapeutic agents in a variety of clinical conditions such as cancer.

In aspects, the compound of the present disclosure comprises a compound represented by a formula:

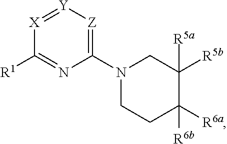

wherein X is selected from N and CR³⁰; and wherein R³⁰ is selected from hydrogen, halogen, amino, hydroxy, cyano, nitro, —SF⁵, aryl, C1-C3 alkyl, C1-C3 alkoxy, C1-C3 aminoalkyl, C1-C3 hydroxyalkyl, and C1-C3 haloalkyl; wherein Y is selected from N and CR⁴⁰; wherein R⁴⁰ is selected from hydrogen, halogen, amino, hydroxy, cyano, nitro, —SF⁵, aryl, C1-C3 alkyl, C1-C3 alkoxy, C1-C3 aminoalkyl, C1-C3 hydroxyalkyl, and C1-C3 haloalkyl; or wherein R³⁰ and R⁴⁰ are covalently bonded and, together with the intermediate carbons, comprise an optionally substituted fused ring selected from 5- to 7-membered heteroaryl and 6-membered aryl; wherein Z is selected from N and CR⁵⁰; wherein R⁵⁰ is selected from hydrogen, halogen, amino, hydroxy, cyano, nitro, —SF⁵, aryl, C1-C3 alkyl, C1-C3 alkoxy, C1-C3 aminoalkyl, C1-C3 hydroxyalkyl, and C1-C3 haloalkyl; or wherein R⁴⁰ and R⁵⁰ are covalently bonded and, together with the intermediate carbons, comprise an optionally substituted fused ring selected from 5- to 7-membered heteroaryl and 6-membered aryl; wherein R¹ is selected from hydrogen, halogen, amino, hydroxy, cyano, nitro, —SF⁵, C1-C3 alkyl, C1-C3 alkoxy, C1-C3 aminoalkyl, C1-C3 hydroxyalkyl, C1-C3 haloalkyl; wherein each of R^{5a} and R^{6a} is independently selected from hydrogen, C1-C6 hydoxyalkyl, C1-C6 aminoalkyl, and C1-C6 haloalkyl; and wherein each of R^{5b} and R^{6b} is independently selected from hydrogen, —(C1-C8 alkanediyl)-aryl, —(C1-C8 alkanediyl)-heteroaryl, —(C1-C4 alkanediyl)-O—(C1-C4 alkanediyl)-aryl, and —(C1-C4 alkanediyl)-O—(C1-C4 alkanediyl)-heteroaryl provided that at least one one of R^{5b} and R^{6b} is not hydrogen; wherein the aryl is optionally substituted with 1, 2, or 3 groups selected from halogen, C1-C3 alkyl, and C1-C3 haloalkyl; and wherein the heteroaryl is optionally substituted with 1, 2, or 3 groups selected from halogen, C1-C3 alkyl, and C1-C3 haloalkyl; or a pharmaceutically acceptable salt thereof.

In a further aspect, the disclosed compounds have a structure represented by a formula:

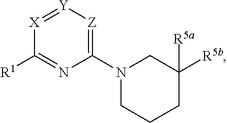

wherein X is selected from N and CR³⁰; and wherein R³⁰ is selected from hydrogen, halogen, amino, hydroxy, cyano, nitro, —SF⁵, aryl, C1-C3 alkyl, C1-C3 alkoxy, C1-C3 aminoalkyl, C1-C3 hydroxyalkyl, and C1-C3 haloalkyl; wherein Y is selected from N and CR⁴⁰; wherein R⁴⁰ is selected from hydrogen, halogen, amino, hydroxy, cyano, nitro, —SF⁵, aryl, C1-C3 alkyl, C1-C3 alkoxy, C1-C3 aminoalkyl, C1-C3 hydroxyalkyl, and C1-C3 haloalkyl; or wherein R³⁰ and R⁴⁰ are covalently bonded and, together with the intermediate carbons, comprise an optionally substituted fused ring selected from 5- to 7-membered heteroaryl and 6-membered aryl; wherein Z is selected from N and CR⁵⁰; wherein R⁵⁰ is selected from hydrogen, halogen, amino, hydroxy, cyano, nitro, —SF⁵, aryl, C1-C3 alkyl, C1-C3 alkoxy, C1-C3 aminoalkyl, C1-C3 hydroxyalkyl, and C1-C3 haloalkyl; or wherein R⁴⁰ and R⁵⁰ are covalently bonded and, together with the intermediate carbons, comprise an optionally substituted fused ring selected from 5- to 7-membered heteroaryl and 6-membered aryl; wherein R¹ is selected from hydrogen, halogen, amino, hydroxy, cyano, nitro, —SF⁵, C1-C3 alkyl, C1-C3 alkoxy, C1-C3 aminoalkyl, C1-C3 hydroxyalkyl, C1-C3 haloalkyl; wherein R^{5a} is selected from hydrogen, C1-C6 hydoxyalkyl, C1-C6 aminoalkyl, and C1-C6 haloalkyl; and wherein R^{5b} is selected from hydrogen, —(C1-C8 alkanediyl)-aryl, —(C1-C8 alkanediyl)-heteroaryl, —(C1-C4 alkanediyl)-O—(C1-C4 alkanediyl)-aryl, and —(C1-C4 alkanediyl)-O—(C1-C4 alkanediyl)-heteroaryl; wherein the aryl is optionally substituted with 1, 2, or 3 groups selected from halogen, C1-C3 alkyl, and C1-C3 haloalkyl; and wherein the heteroaryl is optionally substituted with 1, 2, or 3 groups selected from halogen, C1-C3 alkyl, and C1-C3 haloalkyl; or a pharmaceutically acceptable salt thereof.

In a further aspect, the disclosed compounds have a structure represented by a formula:

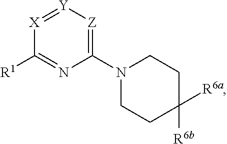

wherein X is selected from N and CR³⁰; and wherein R³⁰ is selected from hydrogen, halogen, amino, hydroxy, cyano, nitro, —SF⁵, aryl, C1-C3 alkyl, C1-C3 alkoxy, C1-C3 aminoalkyl, C1-C3 hydroxyalkyl, and C1-C3 haloalkyl; wherein Y is selected from N and CR⁴⁰; wherein R⁴⁰ is selected from hydrogen, halogen, amino, hydroxy, cyano, nitro, —SF⁵, aryl, C1-C3 alkyl, C1-C3 alkoxy, C1-C3 aminoalkyl, C1-C3 hydroxyalkyl, and C1-C3 haloalkyl; or wherein R³⁰ and R⁴⁰ are covalently bonded and, together with the intermediate carbons, comprise an optionally substituted fused ring selected from 5- to 7-membered heteroaryl and 6-membered aryl; wherein Z is selected from N and CR⁵⁰; wherein R⁵⁰ is selected from hydrogen, halogen, amino, hydroxy, cyano, nitro, —SF⁵, aryl, C1-C3 alkyl, C1-C3 alkoxy, C1-C3 aminoalkyl, C1-C3 hydroxyalkyl, and C1-C3 haloalkyl; or wherein R⁴⁰ and R⁵⁰ are covalently bonded and, together with the intermediate carbons, comprise an optionally substituted fused ring selected from 5- to 7-membered heteroaryl and 6-membered aryl; wherein R¹ is selected from hydrogen, halogen, amino, hydroxy, cyano, nitro, —SF⁵, C1-C3 alkyl, C1-C3 alkoxy, C1-C3 aminoalkyl, C1-C3 hydroxyalkyl, C1-C3 haloalkyl; wherein R^{6a} is selected from hydrogen, C1-C6 hydoxyalkyl, C1-C6 aminoalkyl, and C1-C6 haloalkyl; and wherein R^{6b} is selected from hydrogen, —(C1-C8 alkanediyl)-aryl, —(C1-C8 alkanediyl)-heteroaryl, —(C1-C4 alkanediyl)-O—(C1-C4 alkanediyl)-aryl, and —(C1-C4 alkanediyl)-O—(C1-C4 alkanediyl)-heteroaryl; wherein the aryl is optionally substituted with 1, 2, or 3 groups selected from halogen, C1-C3 alkyl, and C1-C3 haloalkyl; and wherein the heteroaryl is optionally substituted with 1, 2, or 3 groups selected from halogen, C1-C3 alkyl, and C1-C3 haloalkyl; or a pharmaceutically acceptable salt thereof.

Further aspects of the disclosed compounds are as disclosed in the claims of the present disclosure.

It has been found that compounds of the present disclosure interact with the AVIL protein. In cellular assays using glioblastoma (GBM) cell lines and immortalized astrocytes, it was found that representative compounds of the present disclosure provide significantly different IC₅₀ in GBM lines, e.g., a U87 cell-line, as compared to astrocytes. In various aspects, the disclosed compounds can inhibit the interaction of AVIL and F-actin, thereby affecting F-actin dynamics. Moreover, the disclosed compounds can modulate the activity and function of FOXM1 and LIN28B which are downstream proteins affected by the disclosed compounds via AVIL interactions.

In various aspects, it is contemplated herein that the disclosed compounds further comprise their bioisosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prog. Drug Res. 1991, 37, 287-371; (iii) Burger A, “Isosterism and bioanalogy in drug design”, Med. Chem. Res. 1994, 4, 89-92; (iv) Clark R D, Ferguson A M, Cramer R D, “Bioisosterism and molecular diversity”, Perspect. Drug Discovery Des. 1998, 9/10/11, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thornber C W, “Isosterism and molecular modification in drug design”, Chem. Soc. Rev. 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one embodiment, the ester, amide or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, it is contemplated herein that the disclosed compounds further comprise their isotopically-labelled or isotopically-substituted variants, i.e., compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quatemized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### Pharmaceutical Compositions.

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example Eudragit® RS), copolymerizates of acrylic and methacrylic acid esters and trimethyl ammonium methacrylate (for example Eudragit® RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require regulation, limitation, or inhibition of AVIL activity an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for regulating, limiting or inhibiting AVIL activity (e.g., treatment of one or more disorders associated with AVIL dysfunction) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, a polymorph thereof, and a therapeutic agent that can be used to treat disorders or diseases. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional therapeutic agent, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

### Methods of Using the Compounds.

In a further aspect, the present disclosure provides methods of treatment comprising administration of a therapeutically effective amount of a disclosed compound or pharmaceutical composition as disclosed herein above to a subject in need thereof. In particular, the disclosed compounds and disclosed pharmaceutical compositions can be used in methods of treating a disease or disorder that is associated with increased, aberrant, or dysfunctional levels of advillin (AVIL) activity in a cell, tissue, or organism. That is, the disclosed compounds and disclosed pharmaceutical compositions can be used to regulate, limit, or inhibit AVIL activity in a cell, tissue, or organism to provide a clinical or therapeutic benefit to a subject which has been determined to or been diagnosed to have with increased, aberrant, or dysfunctional levels of AVIL activity.

Adult cancers often have complex genomic landscapes, making it challenging to identify key cancer-driving events. As disclosed in International Patent application No. PCT/US2018/057697, which is hereby incorporated herein in its entirety, a pediatric rhabdomyosarcoma was identified as having a gene fusion, AVIL fused to a house-keeping gene, MARS. In adults, AVIL was overexpressed in all of the glioblastomas tested herein below. Tumors were addicted to AVIL dysregulation: silencing the MARS-AVIL fusion in rhabdomyosarcoma, or silencing AVIL in glioblastoma nearly eradicated the cells in culture, and dramatically inhibited in vivo xenografts in mice. Conversely, overexpressing AVIL promoted tumorigenesis. GBM and lower-grade glioma patients with increased AVIL expression had worse prognosis. The effect of AVIL was partly mediated by LIN28B, whose expression also correlated with clinical prognosis. High levels of AVIL expression were also associated with poor patient outcomes in several other cancers. High throughput small molecule screening yielded initial compounds having a phenotypic effect similar to inhibition with siAVIL. As described herein, the present disclosure provides new compounds effective to regulate, limit, or inhibit AVIL activity. That is, in various aspects, the disclosed compounds can inhibit the interaction of AVIL and F-actin, thereby affecting F-actin dynamics. Moreover, the disclosed compounds can modulate the activity and function of FOXM1 and LIN28B which are downstream proteins affected by the disclosed compounds via AVIL interactions.

Oncogene addiction describes a phenomenon according to which tumor cells become reliant on the activity of a particular oncogene and die once this activity is inhibited. (Vivanco, 2014; Weinstein, 2002; Weinstein and Joe, 2006). Many of the targeted cancer therapies exploit this concept (Lord and Ashworth, 2013; Luo et al., 2009). It is perhaps best exemplified by the successful use of imatinib in the therapy of chronic myelogenous leukemia (CML) (Druker et al., 2001). In CML, the major driver of tumorigenesis is the BCR-ABL fusion oncogene; imatinib inhibits the constitutively active BCR-ABL protein kinase, to which leukemic cells become addicted. Other successful examples include trastuzumab targeting ERBB2 addiction (Paik et al., 2008), and vemurafenib targeting BRAF addiction (Bollag et al., 2010; Chapman et al., 2011; Davies et al., 2002). The challenge is to find such key oncogenes. Even though large sets of genome and transcriptome data are available to facilitate the identification of driver mutations in cancer, true signals are often buried in a large number of passenger events.

Glioblastoma (GBM) is the most common primary brain tumor and among the deadliest of human cancers. Despite advances in surgery, radiation and chemotherapy, survival of patients affected by GBM remains dismal (˜15 months after diagnosis). (Prados and Levin, 2000; Castro et al., 2003; King et al., 2005; Stupp et al., 2005). Clearly, better treatment options, and identification of novel therapeutic targets are urgently needed.

Tumor cells use multiple “tricks” to dysregulate some oncogenes, which at the same time give credence to the genes as key players in tumorigenesis and malignancy. However, this knowledge is usually accumulated over a long period of time and often involves different laboratories examining various types of cancer. This concept can be used proactively to find key oncogenes that are dysregulated by multiple mechanisms in different types of cancer. Most adult solid tumors have a complex landscape of genetic lesions, impeding analysis. In contrast, pediatric tumors tend to have fewer point mutations and structural changes. As described in PCT/US2018/057697, our study was initiated in the pediatric tumor, rhabdomyosarcoma. As disclosed therein, a gene fusion was described that results in the juxtaposition of a house-keeping gene next to the AVIL gene, in particular, it was discovered that a subset of GBMs retain AVIL amplification. Interestingly, at RNA and protein levels, all of the GBM cases in our collection overexpress AVIL. Loss-of-function experiments proved the dependency of tumor growth on AVIL dysregulation, yet no effect on non-cancer astrocytes was observed. Consistently, forced overexpression of AVIL resulted in enhanced tumorigenesis. Clinically, higher expression of AVIL correlates with worse patient outcome in GBMs as well as in lower-grade gliomas. The oncogenic effect is at least partly mediated by LIN28B in gliomas. In addition to gliomas, lung cancer, bladder cancer, and renal cancer patients with high level of AVIL expression also had worse prognosis. GBM cells treated with the disclosed compounds have a phenotype similar to the siAVIL transfected cells. One compound was also tested effective in xenografts. AVIL, it was surprisingly discovered, was an oncogene and an effective therapeutic target.

Glioblastoma (GBM), WHO classification Grade IV Astrocytoma, is the most common, and most aggressive malignant primary brain tumor in humans (Dunn et al., 2012). Survival of patients affected by GBM has remained low, despite advances in surgery, radiation, and chemotherapy (Castro et al., 2003; King et al., 2005; Prados and Levin, 2000; Stupp et al., 2005). About 50% of patients diagnosed with GBM die within one year, and 90% die within three years (American Brain Tumor Association, 2014). Our previous studies showed that AVIL is overexpressed in the vast majority, if not all of human glioblastomas. Moreover, it was found that GBM cells depend on the overexpression of AVIL for increased survival and migration. Silencing AVIL induced GBM cell death in vitro, and prevented GBM xenograft formation and growth in animal models. Silencing AVIL also dramatically changed cell morphology, and reduced cell migration/invasion ability. In contrast, normal astrocytes express very low levels of AVIL, and silencing AVIL had no obvious effect on cell growth, or morphology. This demonstrated that AVIL is a new and promising selective therapeutic target, inhibition of which may effectively suppress GBM growth and invasion, yet spare normal brain cells. Indeed, the previous lead compounds had significantly different IC₅₀ values in GBMs versus in astrocytes. In vivo, with only three injections for compound A, smaller tumors were observed as compared to the solvent control. No obvious side effects were observed in the animals. Discovery and optimization of new chemicals that are more potent, stable, water soluble, and capable of crossing the blood-brain barrier led to discovery of the compounds of the present disclosure.

In some aspects, the method includes diagnosis of the subject's need for treatment prior to administration of the compound or pharmaceutical composition. In some aspects, the subject has been diagnosed with a disorder treatable by regulation, limitation, or inhibition of AVIL prior to administering. In some aspects, the subject has been diagnosed with a cancer. In some aspects, the method includes identifying a subject's need for treatment prior to the administering step.

The disclosed compounds can be used as single agents or in combination with one or more other drugs in the treatment, prevention, control, amelioration or reduction of risk of the aforementioned diseases, disorders and conditions for which compounds or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

AVIL is known as a member of the vilin/gelsolin family, which regulates actin filament reorganization (Marks et al., 1998). In encodes a protein also called advillin, which is known to affect cell movement and has been reported to be involved in the formation of filopedia-like structures in fibroblasts, as well as a role in ciliogenesis (Morin et al., 2010). AVIL is overexpressed in in many, if not all, glioblastomas. GBM cells depend on the overexpression of AVIL for increased survival and migration. Silencing AVIL induced GBM cell death in vitro, and can prevent GBM xenograft formation and growth in animal models. Silencing AVIL can also change cell morphology in GBM cells, and reduce cell migration/invasion ability. In contrast, normal astrocytes express very low levels of AVIL, and silencing AVIL had no obvious effect on cell growth, or morphology.

Therefore, compounds that regulate, limit, or inhibit AVIL expression show improved prognosis in the treatment of cancers in which there is an inverse correlation between AVIL expression and patient prognosis, such as, but not limited to, brain cancer and cancerous tumors such as glioblastomas, rhabdosarcomas, gliomas, lung cancer, bladder cancer including bladder urothelial carcinoma, and renal cancer including kidney clear cell carcinoma.

Accordingly, in various aspects, the present disclosure pertains to methods of targeting AVIL with the disclosed compounds. The disclosed compounds or disclosed pharmaceutical compositions can act as regulators of AVIL expression in cells having increased, aberrant, or dysfunctional levels of AVIL, and accordingly can be useful in the treatment of cancer (including but not limited to those types mentioned herein).

The compositions can be administered alone or combination with a chemotherapeutic drug. Combination therapy can present advantages over single-agent therapies: lower treatment failure rate, lower case-fatality ratios, slower development of resistance and consequently, less money needed for the development of new drugs. Chemotherapeutic drugs include conventional chemotherapeutic reagents such as alkylating agents, anti-metabolites, anti-mitototics, plant alkaloids, antibiotics, and miscellaneous compounds. Examples of these drugs include CDDP, methotrexate, vincristine, adriamycin, bleomycin, carmustine, hydroxyurea, hydrazine, nitrosoureas, triazenes such as dacarabzine and temozolomide, nitrogen mustards such as chlorambucil, cyclophosphamide, isofamide, mechlorethamine, melphalan, uracil mustard; aziridine such as thiotepa; methanesulphonate esters such as busulfan; platinum complexes such as cisplatin, carboplatin; bioreductive alkylators, such as mitomycin and and altretemine. Chemotherapeutic drugs also include proteasome inhibitors such as salinosporamides, bortezomib, PS-519, and omuralide. The disclosed compounds can also be administered in combination with surgery. For example, the disclosed compounds can be administered prior to, during or after surgery or radiotherapy. Administration during surgery can be as a bathing solution for the operation site. The resected tumor can also be bathed in the disclosed compounds.

In further aspects, the disclosed compounds can be utilized in combination with one or more chemotherapeutic agents. For example, the one or more chemotherapeutic agent can be a chemotherapeutic agent selected from alkylating agents, antimetabolites, platinating agents, toxoids, EGFR inhibitors, anti-hormonal agents, topoisomerase inhibitors, tubulin agents, signaling inhibitors (e.g., kinase inhibitors), and other chemotherapeutic agents.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1l and calicheamicin omegal1 (see, e.g., Agnew, Chem Intl. Ed. Engl., 33:183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin, and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovovin; novantrone; edatrexate; daunomycin; aminopterin; ibandronate; topoisomerase inhibitor RFS 2000; difluorometlhylornithine (DMFO); retinoids such as retinoic acid, including bexarotene (TARGRETIN®); bisphosphonates such as clodronate (for example, BONEFOSO® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (Pfizer); perifosine, COX-2 inhibitor (e.g., celecoxib or etoricoxib), proteosome inhibitor (e.g., PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Herein, chemotherapeutic agents include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

Herein, a “taxoid” is a chemotherapeutic agent that functions to inhibit microtubule depolymerization. Examples include paclitaxel (TAXOL®), albumin-engineered nanoparticle formulation of paclitaxel (ABRAXANE™), and docetaxel (TAXOTERE®). The preferred taxoid is paclitaxel.

As used herein, the term “EGFR inhibitor” refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al., Eur. J. Cancer, 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3 and E7.6.3 and described in U.S. Pat. No. 6,235,883; MDX-447 (Medarex Inc); and mAb 806 or humanized mAb 806 (Johns et al., J. Biol. Chem., 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)pr opoxy]-6-quinazolinyl]-, dihydrochloride, Pfizer Inc.); ZD1839, gefitinib (IRESSA J) 4-(3′-Chloro-4′-fluoroanilino)-7-methoxy-6-(3-morpholino propoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-qu inolinyl]-4-(dimethylamino)-2-butenamide) (Wyeth); AG1478 (Sugen); AG1571 (SU 5271; Sugen); dual EGFR/HER2 tyrosine kinase inhibitors such as lapatinib (GW 572016 or N-[3-chloro-4-[(3fluorophenyl)methoxy]phenyl]6[5[[[2methylsulfonyl) ethyl]amino]methyl]-2-furanyl]-4-quinazolinamine; Glaxo-SmithKline).

A “tyrosine kinase inhibitor” is a molecule which inhibits tyrosine kinase activity of a tyrosine kinase such as a HER receptor. Examples of such inhibitors include the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GW572016; available from Glaxo-SmithKline) an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibits Raf-1 signaling; non-HER targeted TK inhibitors such as Imatinib mesylate (GLEEVAC J) available from Glaxo; MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); Imatinib mesylate (Gleevac; Novartis); PKI 166 (Novartis); GW2016 (Glaxo SmithKline); CI-1033 (Pfizer); EKB-569 (Wyeth); Semaxinib (Sugen); ZD6474 (AstraZeneca); PTK-787 (Novartis/Schering AG); INC-1C11 (Imclone); or as described in any of the following patent publications: U.S. Pat. No. 5,804,396; WO99/09016 (American Cyanamid); WO98/43960 (American Cyanamid); WO97/38983 (Warner Lambert); WO99/06378 (Warner Lambert); WO99/06396 (Warner Lambert); WO96/30347 (Pfizer, Inc); WO96/33978 (Zeneca); WO96/3397 (Zeneca); and WO96/33980 (Zeneca).

Thus in various further aspects, the disclosed compounds can be administered to subjects in combination with one or more chemotherapeutic drugs. For example, treating a subject with a glioma can be effected by a method comprising administering to the subject a disclosed compound or a pharmaceutically acceptable salt or hydrate thereof in combination with one or more chemotherapeutic drugs. For example, inhibiting intracranial metastasis of gliomal cancer cells in a subject can be effected by a method comprising administering to the subject a disclosed compound or a pharmaceutically acceptable salt or hydrate thereof in combination with one or more chemotherapeutic drugs. For example, preventing relapse of glioma in a subject can be effected by a method comprising administering to the subject a disclosed compound or a pharmaceutically acceptable salt or hydrate thereof in combination with one or more chemotherapeutic drugs.

It is contemplated that the disclosed compounds can be administered before, simultaneously, or after the administration of one or more chemotherapeutic drugs. While not wishing to be bound by theory, it is believed that the disclosed compounds, in combination with one or more chemotherapeutic drugs, can have an augmented or synergistic effect on the subject. Further, disclosed compounds, in combination with one or more chemotherapeutic drugs, can be individually given in dosages lower than the one or more chemotherapeutic drugs would be typically administered as single-agent therapies.

In further aspects, the invention relates to administration of the disclosed compounds to subjects in combination with temozolomide (4-methyl-5-oxo-2,3,4,6,8-pentazabicyclo [4.3.0] nona-2,7,9-triene-9-carboxamide). For example, treating a subject with a glioma can be effected by a method comprising administering to the subject a disclosed compound or a pharmaceutically acceptable salt or hydrate thereof in combination with temozolomide. For example, inhibiting intracranial metastasis of gliomal cancer cells in a subject can be effected by a method comprising administering to the subject a disclosed compound or a pharmaceutically acceptable salt or hydrate thereof in combination with temozolomide. For example, preventing relapse of glioma in a subject can be effected by a method comprising administering to the subject a disclosed compound or a pharmaceutically acceptable salt or hydrate thereof in combination with Temozolomide.

It is also understood that the disclosed compounds, when administered to subjects in combination with one or more chemotherapeutic drugs, can also be employed in connection with radiation therapy and/or surgical therapy.

Radiation therapy (Radiotherapy), including brachytherapy, can be used to treat gliomas. In one aspect, the invention relates to the administration of the disclosed compounds to subjects in connection with radiation therapy. It is contemplated that the disclosed compounds can be administered before, during, or after the radiation therapy. For example, treating a subject with a glioma can be effected by a method comprising administering to the subject a disclosed compound or a pharmaceutically acceptable salt or hydrate thereof in connection with radiation therapy. For example, inhibiting intracranial metastasis of gliomal cancer cells in a subject can be effected by a method comprising administering to the subject a disclosed compound or a pharmaceutically acceptable salt or hydrate thereof in connection with radiation therapy. For example, preventing relapse of glioma in a subject can be effected by a method comprising administering to the subject a disclosed compound or a pharmaceutically acceptable salt or hydrate thereof in connection with radiation therapy.

While not wishing to be bound by theory, it is believed that the disclosed compounds, in combination with radiotherapy, can have an augmented or synergistic effect in a subject. Further, the disclosed compounds, when used in combination with radiotherapy, can lower a subject's need for radiotherapy (e.g., less radiation need be used) and/or can lower a subject's need for disclosed compounds (e.g., disclosed compounds can be given in dosages lower than would be typically administered as single-agent therapies).

It is also understood that the disclosed compounds, when administered to subjects in connection with radiation therapy, can also be employed in combination with one or more chemotherapeutic drugs and/or in connection surgical therapy.

Surgery can be used to treat gliomas. In one aspect, the invention relates to the administration of the disclosed compounds to subjects in connection with surgical treatment. For example, treating a subject with a glioma can be effected by a method comprising administering to the subject a disclosed compound or a pharmaceutically acceptable salt or hydrate thereof in connection with surgery. For example, inhibiting intracranial metastasis of gliomal cancer cells in a subject can be effected by a method comprising administering to the subject a disclosed compound or a pharmaceutically acceptable salt or hydrate thereof in connection with surgery. For example, preventing relapse of glioma in a subject can be effected by a method comprising administering to the subject a disclosed compound or a pharmaceutically acceptable salt or hydrate thereof in connection with surgery.

It is contemplated that the disclosed compounds can be administered before, during, or after surgical treatment. While not wishing to be bound by theory, it is believed that the disclosed compounds, in combination with surgery, can have an augmented or synergistic effect on the subject. Further, disclosed compounds, when used in combination with surgery, can lower a subject's need for surgery (e.g., less tissue need be removed) and/or can lower a subject's need for disclosed compounds (e.g., disclosed compounds can be given in dosages lower than would be typically administered as single-agent therapies).

It is also understood that the disclosed compounds, when administered to subjects in connection with surgical therapy, can also be employed in connection with radiation therapy and/or surgical therapy.

The disclosed compositions can also be employed to prevent relapse in a subject previously treated for a glioma. In one aspect, such a method comprises administering to the subject a prophylactically effective amount of a disclosed compound or a pharmaceutically acceptable salt or hydrate thereof. It is understood that the dosage needed to prevent relapse (i.e. maintenance dose) may be less (e.g., half) of the dosage needed to effect treatment of a glioma. Thus, in maintenance, a suitable dosage of the disclosed compound or a pharmaceutically acceptable salt or hydrate thereof can be from 0.5 to about 250 mg/kg of the subject, can be administered at a dosage of from 5 to about 100 mg/kg of the subject, can be administered at a dosage of from 5 to about 50 mg/kg of the subject, or can be administered at a dosage of from 10 to about 250 mg/kg of the subject.

It is also understood that when using the disclosed compounds for preventing of relapse of glioma, in either single agent therapy or in combination therapy, can be also administered to subjects in connection with surgical therapy and/or surgical therapy.

In a further aspect, the present disclosure pertains to methods of inhibiting AVIL activity in a subject, comprising administering to the subject a disclosed compound, or a pharmaceutically acceptable salt thereof, wherein the disclosed compound comprises one or more disclosed compounds.

In a further aspect, the present disclosure pertains to methods of treating a subject with a glioma, comprising administering to the subject a disclosed compound, or a pharmaceutically acceptable salt thereof, wherein the disclosed compound comprises one or more disclosed compounds.

In a further aspect, the present disclosure pertains to methods of preventing relapse in a subject previously treated for a glioma, comprising administering to the subject a disclosed compound, or a pharmaceutically acceptable salt thereof, wherein the disclosed compound comprises one or more disclosed compounds.

In a further aspect, the cancer is a cancer of the brain. In a still further aspect, the cancer is selected from acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor cell.

In a further aspect, the cancer is a glioma. In a still further aspect, the glioma is glioblastoma multiforme. In a yet further aspect, the glioma is selected from is selected from a ependymoma, astrocytoma, oligodendroglioma, and oligoastrocytoma. In a yet further aspect, the glioma is selected from a juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, paraganglioma, and ganglioglioma cell.

Cancers that may be treated by a disclosed compound include, but are not limited to: cardiac; sarcoma (angiosarcoma, fibrosarcoma, rhabdomyosarcoma, liposarcoma); myxoma; rhabdomyoma; fibroma; lipoma and teratoma; lung, e.g., non-small cell lung, bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal, e.g., esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), colon, colorectal, rectal; genitourinary tract, e.g., kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, e.g., hepatoma (hepatocellular carcinoma), cholangiocarcinoma, hepatoblastoma, angiosarcoma, hepatocellular adenoma, hemangioma; bone, e.g., osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, e.g., skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); gynecological, e.g., uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); hematologic, e.g., blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; skin, e.g., malignant melanoma, basal cell carcinoma, squamous cell carcinoma, Karposi's sarcoma, moles dysplastic nevi, lipoma, angioma, dermatofibroma, keloids, psoriasis; and adrenal glands, e.g., a neuroblastoma. Thus, the term “cancerous cell” as provided herein, includes a cell afflicted by any one of the above-identified conditions.

Cancers that may be treated by a disclosed compound include, but are not limited to, any of the following cancers in which it has been determined that the cancer is associated with aberrant expression of AVIL: cardiac; sarcoma (angiosarcoma, fibrosarcoma, rhabdomyosarcoma, liposarcoma); myxoma; rhabdomyoma; fibroma; lipoma and teratoma; lung, e.g., non-small cell lung, bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal, e.g., esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), colon, colorectal, rectal; genitourinary tract, e.g., kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, e.g., hepatoma (hepatocellular carcinoma), cholangiocarcinoma, hepatoblastoma, angiosarcoma, hepatocellular adenoma, hemangioma; bone, e.g., osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, e.g., skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); gynecological, e.g., uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); hematologic, e.g., blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; skin, e.g., malignant melanoma, basal cell carcinoma, squamous cell carcinoma, Karposi's sarcoma, moles dysplastic nevi, lipoma, angioma, dermatofibroma, keloids, psoriasis; and adrenal glands, e.g., a neuroblastoma. Thus, the term “cancerous cell” as provided herein, includes a cell afflicted by any one of the above-identified conditions.

### Kits.

In a further aspect, the present disclosure relates to kits comprising at least one disclosed compound, or a pharmaceutically acceptable salt, hydrate, solvate, or polymorph thereof, and one or more of: (a) at least one agent known to treat a cancer; (b) instructions for treating a cancer and/or instructions for administering the compound in connection with the treatment of cancer. The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

### Research Tools.

The disclosed compounds and pharmaceutical compositions have activity as inhibitors of AVIL expression. As such, the disclosed compounds are also useful as research tools. Accordingly, one aspect of the present disclosure relates to a method of using a compound of the invention as a research tool, the method comprising conducting a biological assay using a compound of the invention. Compounds of the invention can also be used to evaluate new chemical compounds. Thus another aspect of the invention relates to a method of evaluating a test compound in a biological assay, comprising: (a) conducting a biological assay with a test compound to provide a first assay value; (b) conducting the biological assay with a compound of the invention to provide a second assay value; wherein step (a) is conducted either before, after or concurrently with step (b); and (c) comparing the first assay value from step (a) with the second assay value from step (b). Exemplary biological assays include an assay that can be conducted in vitro or in a cell culture system. Still another aspect of the invention relates to a method of studying a biological system, e.g., a model animal for a clinical condition, or biological sample having increased, aberrant, or dysfunctional levels of AVIL, the method comprising: (a) contacting the biological system or sample with a compound of the invention; and (b) determining the effects caused by the compound on the biological system or sample.

### Aspects.

The following listing of exemplary aspects supports and is supported by the disclosure provided herein.

Aspect 1. A compound having a structure represented by a formula:

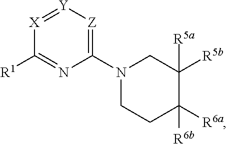

wherein X is selected from N and CR³⁰; and wherein R³⁰ is selected from hydrogen, halogen, amino, hydroxy, cyano, nitro, —SF₅, aryl, C1-C3 alkyl, C1-C3 alkoxy, C1-C3 aminoalkyl, C1-C3 hydroxyalkyl, and C1-C3 haloalkyl; wherein Y is selected from N and CR⁴⁰; wherein R⁴⁰ is selected from hydrogen, halogen, amino, hydroxy, cyano, nitro, —SF₅, aryl, C1-C3 alkyl, C1-C3 alkoxy, C1-C3 aminoalkyl, C1-C3 hydroxyalkyl, and C1-C3 haloalkyl; or wherein R³⁰ and R⁴⁰ are covalently bonded and, together with the intermediate carbons, comprise an optionally substituted fused ring selected from 5- to 7-membered heteroaryl and 6-membered aryl; wherein Z is selected from N and CR⁵⁰; wherein R⁵⁰ is selected from hydrogen, halogen, amino, hydroxy, cyano, nitro, —SF₅, aryl, C1-C3 alkyl, C1-C3 alkoxy, C1-C3 aminoalkyl, C1-C3 hydroxyalkyl, and C1-C3 haloalkyl; or wherein R⁴⁰ and R⁵⁰ are covalently bonded and, together with the intermediate carbons, comprise an optionally substituted fused ring selected from 5- to 7-membered heteroaryl and 6-membered aryl; wherein R¹ is selected from hydrogen, halogen, amino, hydroxy, cyano, nitro, —SF₅, C1-C3 alkyl, C1-C3 alkoxy, C1-C3 aminoalkyl, C1-C3 hydroxyalkyl, C1-C3 haloalkyl; wherein each of R^{5a} and R^{6a} is independently selected from hydrogen, C1-C6 hydoxyalkyl, C1-C6 aminoalkyl, and C1-C6 haloalkyl; and wherein each of R^{5b} and R^{6b} is independently selected from hydrogen, —(C1-C8 alkanediyl)-aryl, —(C1-C8 alkanediyl)-heteroaryl, —(C1-C4 alkanediyl)-O—(C1-C4 alkanediyl)-aryl, and —(C1-C4 alkanediyl)-O—(C1-C4 alkanediyl)-heteroaryl provided that at least one one of R^{5b} and R^{6b} is not hydrogen; wherein the aryl is optionally substituted with 1, 2, or 3 groups selected from halogen, C1-C3 alkyl, and C1-C3 haloalkyl; and wherein the heteroaryl is optionally substituted with 1, 2, or 3 groups selected from halogen, C1-C3 alkyl, and C1-C3 haloalkyl; or a pharmaceutically acceptable salt thereof.

Aspect 2. The compound of Aspect 1, wherein X is N.

Aspect 3. The compound of Aspect 2, wherein Y is CR⁴⁰; and wherein Z is CR⁵⁰.

Aspect 4. The compound of Aspect 2, wherein each of Y and Z is CH.

Aspect 5. The compound of Aspect 2, wherein Y is CR⁴⁰; and wherein Z is CH.

Aspect 6. The compound of Aspect 2, wherein Y is CH; and wherein Z is CR⁵⁰.

Aspect 7. The compound of Aspect 1, wherein X is N; and wherein Y is N.

Aspect 8. The compound of Aspect 7, wherein Z is CR⁵⁰.

Aspect 9. The compound of Aspect 7, wherein Z is CH.

Aspect 10. The compound of Aspect 1, wherein X is N; and wherein Z is N.

Aspect 11. The compound of Aspect 7, wherein Y is CR⁵⁰.

Aspect 12. The compound of Aspect 7, wherein Y is CH.

Aspect 13. The compound of Aspect 1, wherein R³⁰ and R⁴⁰ are covalently bonded and, together with the intermediate carbons, comprise an optionally substituted fused ring selected from 5- to 7-membered heteroaryl and 6-membered aryl.

Aspect 14. The compound of Aspect 13, wherein the fused ring is a 6-membered aryl ring.

Aspect 15. The compound of Aspect 1, wherein R⁴⁰ and R⁵⁰ are covalently bonded and, together with the intermediate carbons, comprise an optionally substituted fused ring selected from 5- to 7-membered heteroaryl and 6-membered aryl.

Aspect 16. The compound of Aspect 15, wherein the fused ring is a 6-membered aryl ring.

Aspect 17. The compound of any one of Aspects 1-16, wherein R¹ is selected from hydrogen, halogen, amino, hydroxy, cyano, nitro, —SF₅, methyl, —NHCH3, —N(CH3)2, —OCH3, —CH2OH, and C1 haloalkyl.

Aspect 18. The compound of Aspect 10, wherein R¹ is selected from halogen, amino, hydroxy, cyano, nitro, —SF₅, methyl, —NHCH3, —N(CH3)2, —OCH3, —CH2OH, and C1 haloalkyl.

Aspect 19. The compound of Aspect 10, wherein R¹ is selected from fluoro, chloro, amino, hydroxy, cyano, nitro, —SF₅, methyl, —NHCH3, —N(CH3)2, —OCH3, —CH2OH, —CH2F, and —CH2Cl.

Aspect 20. The compound of Aspect 10, wherein R¹ is selected from amino, —NHCH3, and —N(CH3)2.

Aspect 21. The compound of any one of Aspects 1-20, wherein each of R^{5a}, R^{5b} and R^{6a} are hydrogen; and wherein R^{6b} is selected from —(C1-C3 alkanediyl)-O—(C1-C3 alkanediyl)-aryl and —(C1-C3 alkanediyl)-O—(C1-C3 alkanediyl)-heteroaryl.

Aspect 22. The compound of Aspect 25, wherein the aryl is unsubstituted; or wherein the heteroaryl is unsubstituted.

Aspect 23. The compound of Aspect 25 or 26, wherein the aryl is phenyl; or wherein the heteroaryl is pydrinyl, pyrimidinyl, pyrazinyl, or triazinyl.

Aspect 24. The compound of any one of Aspects 25-31, wherein R^{6b} is selected from —(C3 alkanediyl)-O—(C3 alkanediyl)-aryl and —(C1-C3 alkanediyl)-O—(C1-C3 alkanediyl)-heteroaryl.

Aspect 25. The compound of any one of Aspects 1-20, wherein each of R^{5a} and R^{5b}are hydrogen; wherein R^{6a} is selected from hydrogen, C1-C3 hydoxyalkyl, C1-C3 aminoalkyl, and C1-C3 haloalkyl; and wherein R^{6b} is selected from —(C1-C6 alkanediyl)-aryl, —(C1-C6 alkanediyl)-heteroaryl, —(C1-C3 alkanediyl)-O—(C1-C3 alkanediyl)-aryl, and —(C1-C3 alkanediyl)-O—(C1-C3 alkanediyl)-heteroaryl.

Aspect 26. The compound of Aspect 25, wherein the aryl is unsubstituted; or wherein the heteroaryl is unsubstituted.

Aspect 27. The compound of Aspect 25 or 26, wherein the aryl is phenyl; or wherein the heteroaryl is pydrinyl, pyrimidinyl, pyrazinyl, or triazinyl.

Aspect 28. The compound of any one of Aspects 25-27, wherein R^{6a} is selected from methyl, ethyl, —CH₂F, —CH₂Cl, —CH₂Br, —CH₂I, —CH₂CH₂F, —CH₂CH₂Cl, —CH₂CH₂Br, —CH₂CH₂I, —CHF₂, —CF₃, —CHCl₂, —CCl₃, —CHBr₂, —CBr₃, —CHI₂, —Cl₃, —CH₂CHF₂, —CH₂CF₃, —CH₂CHCl₂, —CH₂CCl₃, —CH₂CHBr₂, —CH₂CBr₃, —CH₂CHI₂, —CH₂OH, —(CH₂)₂OH, —CH(OH)CH₃, —CH₂NH₂, —CH₂NHCH₃, —CH₂N(CH₃), —(CH₂)₂NH2, —(CH₂)₂NHCH₃, ahd —(CH₂)₂N(CH₃).

Aspect 29. The compound of Aspect 28, wherein R^{6a} is selected from methyl, —CH₂F, —CH₂Cl, —CHF₂, —CF₃, —CHCl₂, —CCl₃, —CH₂OH, —CH(OH)CH₃, —CH₂NH₂, —CH₂NHCH₃, and —CH₂N(CH₃).

Aspect 30. The compound of Aspect 28, wherein R^{6a} is selected from —CH₂OH and —CH(OH)CH₃.

Aspect 31. The compound of Aspect 28, wherein R^{6a} is —CH₂OH.

Aspect 32. The compound of any one of Aspects 25-31, wherein R^{6b} is selected from —(C3-C6 alkanediyl)-aryl, —(C3-C6 alkanediyl)-heteroaryl, —(C3 alkanediyl)-O—(C3 alkanediyl)-aryl, and —(C1-C3 alkanediyl)-O—(C1-C3 alkanediyl)-heteroaryl.

Aspect 33. The compound of Aspect 32, wherein R^{6b} is selected from —(C3-C6 alkanediyl)-phenyl, and —(C3 alkanediyl)-O—(C3 alkanediyl)-phenyl.

Aspect 34. The compound of any one of Aspects 1-20, wherein each of R^{6a} and R^{6b}are hydrogen; wherein R^{5a} is selected from hydrogen, C1-C3 hydoxyalkyl, C1-C3 aminoalkyl, and C1-C3 haloalkyl; and wherein R^{5b} is selected from —(C1-C6 alkanediyl)-aryl, —(C1-C6 alkanediyl)-heteroaryl, —(C1-C3 alkanediyl)-O—(C1-C3 alkanediyl)-aryl, and —(C1-C3 alkanediyl)-O—(C1-C3 alkanediyl)-heteroaryl.

Aspect 35. The compound of Aspect 34, wherein the aryl is unsubstituted; or wherein the heteroaryl is unsubstituted.

Aspect 36. The compound of Aspect 34 or 35, wherein the aryl is phenyl; or wherein the heteroaryl is pydrinyl, pyrimidinyl, pyrazinyl, or triazinyl.

Aspect 37. The compound of any one of Aspects 34-36, wherein R^{5a} is selected from methyl, ethyl, —CH₂F, —CH₂Cl, —CH₂Br, —CH₂I, —CH₂CH₂F, —CH₂CH₂Cl, —CH₂CH₂Br, —CH₂CH₂I, —CHF₂, —CF₃, —CHCl₂, —CCl₃, —CHBr₂, —CBr₃, —CHI₂, —Cl₃, —CH₂CHF₂, —CH₂CF₃, —CH₂CHCl₂, —CH₂CCl₃, —CH₂CHBr₂, —CH₂CBr₃, —CH₂CHI₂, —CH₂OH, —(CH₂)₂OH, —CH(OH)CH₃, —CH₂NH₂, —CH₂NHCH₃, —CH₂N(CH₃), —(CH₂)₂NH2, —(CH₂)₂NHCH₃, ahd —(CH₂)₂N(CH₃).

Aspect 38. The compound of Aspect 37, wherein R^{5a} is selected from methyl, —CH₂F, —CH₂Cl, —CHF₂, —CF₃, —CHCl₂, —CCl₃, —CH₂OH, —CH(OH)CH₃, —CH₂NH₂, —CH₂NHCH₃, and —CH₂N(CH₃).

Aspect 39. The compound of Aspect 37, wherein R^{5a} is selected from —CH₂OH and —CH(OH)CH₃.

Aspect 40. The compound of Aspect 37, wherein R^{5a} is —CH₂OH.

Aspect 41. The compound of any one of Aspects 34-40, wherein R^{5b} is selected from —(C3-C6 alkanediyl)-aryl, —(C3-C6 alkanediyl)-heteroaryl, —(C3 alkanediyl)-O—(C3 alkanediyl)-aryl, and —(C1-C3 alkanediyl)-O—(C1-C3 alkanediyl)-heteroaryl.

Aspect 42. The compound of Aspect 42, wherein R^{5b} is selected from —(C3-C6 alkanediyl)-phenyl, and —(C3 alkanediyl)-O—(C3 alkanediyl)-phenyl.

Aspect 43. The compound of any one of Aspects 1-42, wherein R³⁰ is selected from hydrogen, halogen, amino, C1-C3 alkyl, and C1-C3 haloalkyl.

Aspect 44. The compound of Aspect 43, wherein R³⁰ is selected from methyl, ethyl and C1-C2 haloalkyl.

Aspect 45. The compound of any one of Aspects 1-42, wherein R⁴⁰ is selected from halogen, amino, C1-C3 alkyl, and C1-C3 haloalkyl.

Aspect 46. The compound of Aspect 45, wherein R⁴⁰ is selected from methyl, ethyl and C1-C2 haloalkyl.

Aspect 47. The compound of any one of Aspects 1-42, wherein R⁵⁰ is selected from hydrogen, halogen, amino, C1-C3 alkyl, and C1-C3 haloalkyl.

Aspect 48. The compound of Aspect 46, wherein R⁵⁰ is selected from fluoro, methyl, —CH₂F, —CH₂Cl, —CH₂Br, —CH₂I, —CHF₂, —CF₃, —CHCl₂, —CCl₃, —CHBr₂, —CBr₃, —CHI₂, and —Cl₃.

Aspect 49. The compound of any one of Aspects 1-48, having a structure represented by a formula:

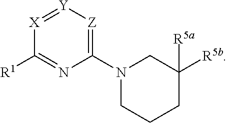

Aspect 50. The compound of Aspect 49, wherein the compound is not present as:

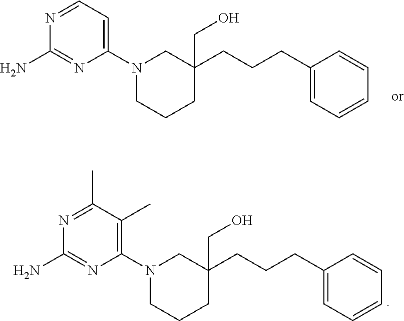 [SMILES: Nc1nccc(N2CCCC(CO)(CCCc3ccccc3)C2)n1 ; Cc1nc(N)nc(N2CCCC(CO)(CCCc3ccccc3)C2)c1C]

Aspect 51. The compound of Aspect 49, having a structure represented by a formula:

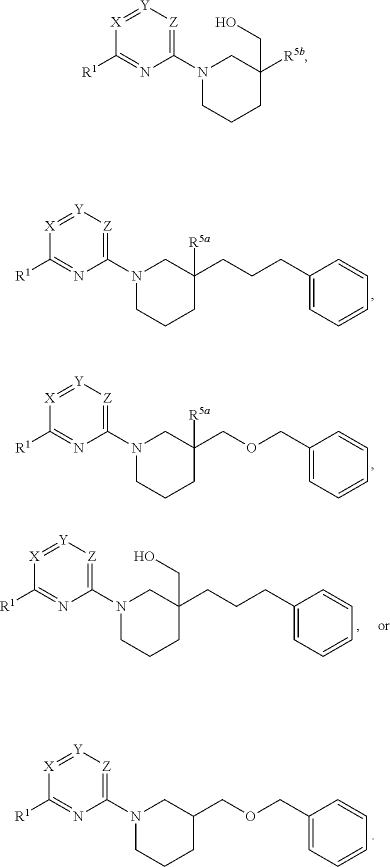

Aspect 52. The compound of Aspect 49, having a structure represented by a formula:

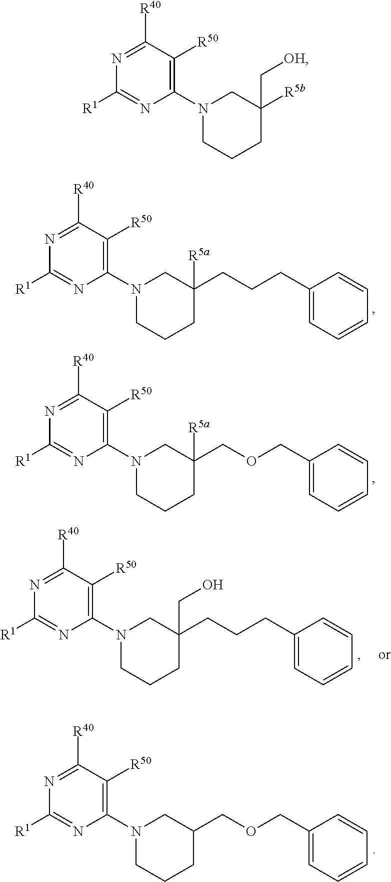

Aspect 53. The compound of Aspect 49, having a structure represented by a formula:

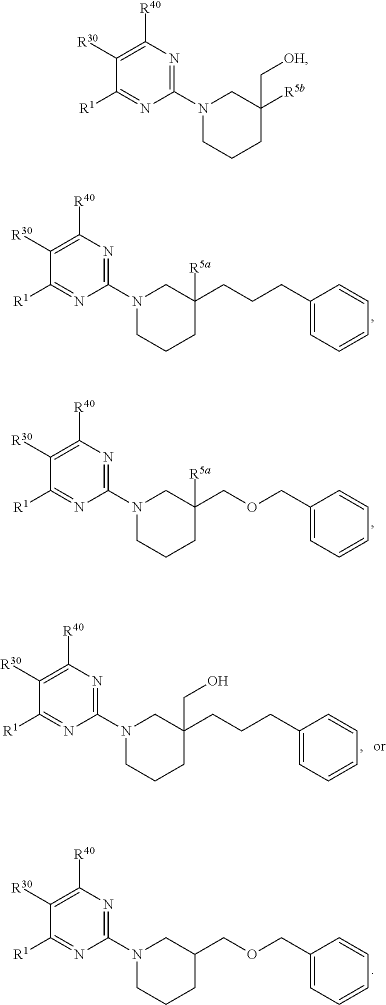

Aspect 54. The compound of Aspect 49, having a structure represented by a formula:

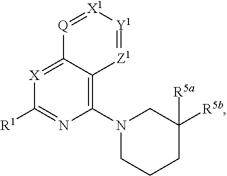

wherein Q is selected from N and CR²⁰; and wherein R²⁰ is selected from hydrogen, halogen, amino, hydroxy, cyano, nitro, —SF₅, C1-C3 alkyl, C1-C3 alkoxy, C1-C3 aminoalkyl, C1-C3 hydroxyalkyl, C1-C3 haloalkyl; wherein X¹ is selected from N and CR³¹; and wherein R³¹ is selected from hydrogen, halogen, amino, hydroxy, cyano, nitro, —SF₅, C1-C3 alkyl, C1-C3 alkoxy, C1-C3 aminoalkyl, C1-C3 hydroxyalkyl, C1-C3 haloalkyl; wherein Y¹ is selected from N and CR⁴¹; and wherein R⁴¹ is selected from hydrogen, halogen, amino, hydroxy, cyano, nitro, —SF₅, C1-C3 alkyl, C1-C3 alkoxy, C1-C3 aminoalkyl, C1-C3 hydroxyalkyl, C1-C3 haloalkyl; and wherein Z¹ is selected from N and CR⁵¹; and wherein R⁵¹ is selected from hydrogen, halogen, amino, hydroxy, cyano, nitro, —SF₅, C1-C3 alkyl, C1-C3 alkoxy, C1-C3 aminoalkyl, C1-C3 hydroxyalkyl, C1-C3 haloalkyl.

Aspect 55. The compound of Aspect 54, wherein Q is N; wherein X¹ is CR³¹; wherein Y¹ is CR⁴¹; and wherein Z¹ is CR⁵¹.

Aspect 56. The compound of Aspect 55, wherein each of X¹, Y¹, and Z¹ is CH.

Aspect 57. The compound of Aspect 54, wherein Q is CR²⁰; wherein X¹ is CR³¹; wherein Y¹ is CR⁴¹; and wherein Z¹ is CR⁵¹.

Aspect 58. The compound of Aspect 57, wherein each of Q, X¹, Y¹, and Z¹ is CH.

Aspect 59. The compound of Aspect 54, having a structure represented by a formula:

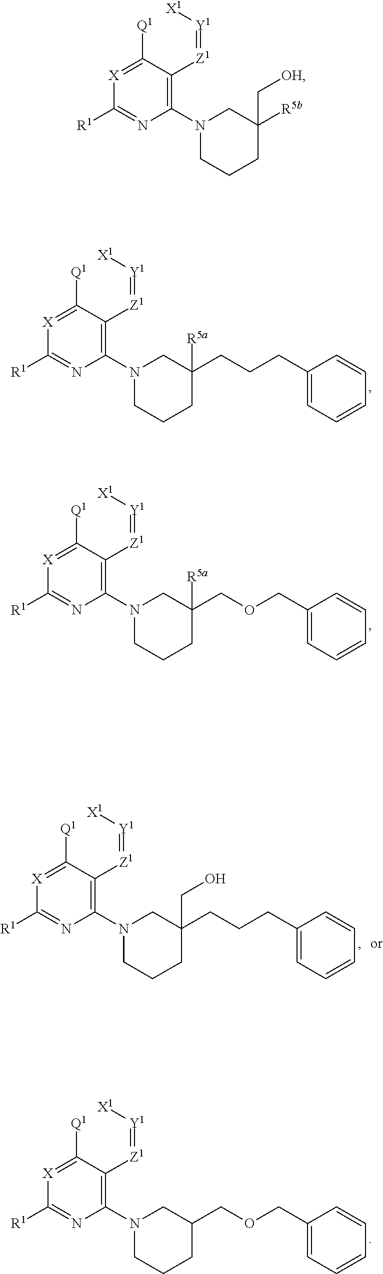

Aspect 60. The compound of any one of Aspects 1-48, having a structure represented by a formula:

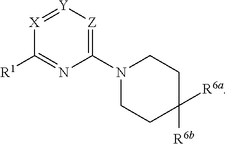

Aspect 61. The compound of Aspect 60, wherein the compound is not present as:

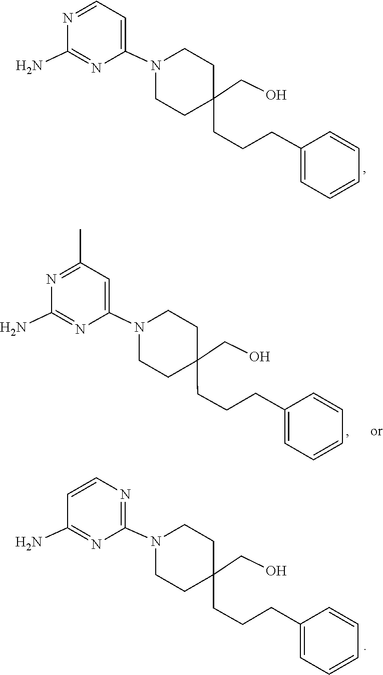 [SMILES: Nc1nccc(N2CCC(CO)(CCCc3ccccc3)CC2)n1 ; Cc1cc(N2CCC(CO)(CCCc3ccccc3)CC2)nc(N)n1 ; Nc1ccnc(N2CCC(CO)(CCCc3ccccc3)CC2)n1]

Aspect 62. The compound of Aspect 60, having a structure represented by a formula:

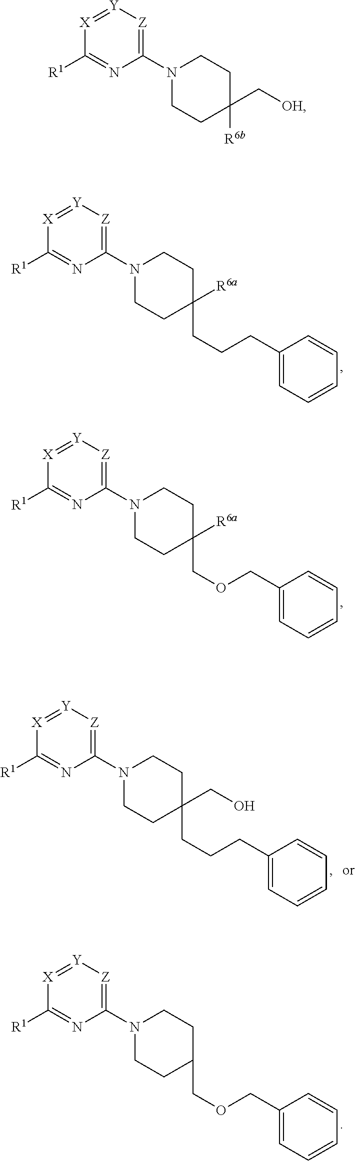

Aspect 63. The compound of Aspect 60, having a structure represented by a formula:

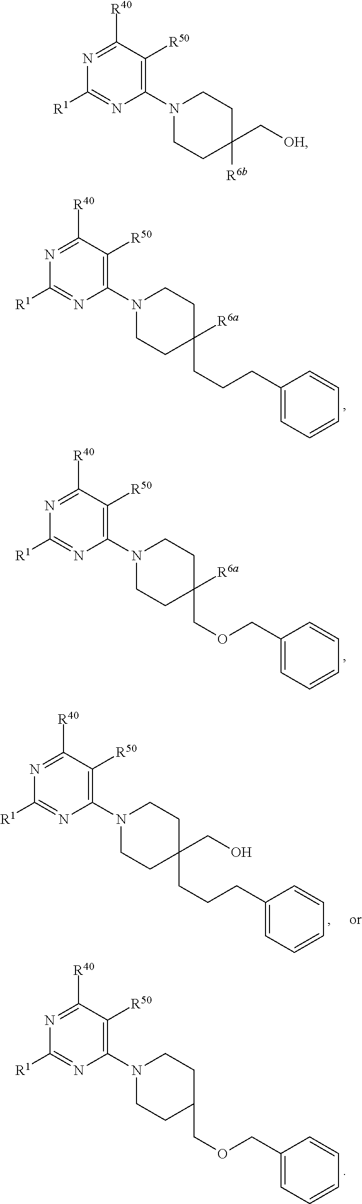

Aspect 64. The compound of Aspect 60, having a structure represented by a formula:

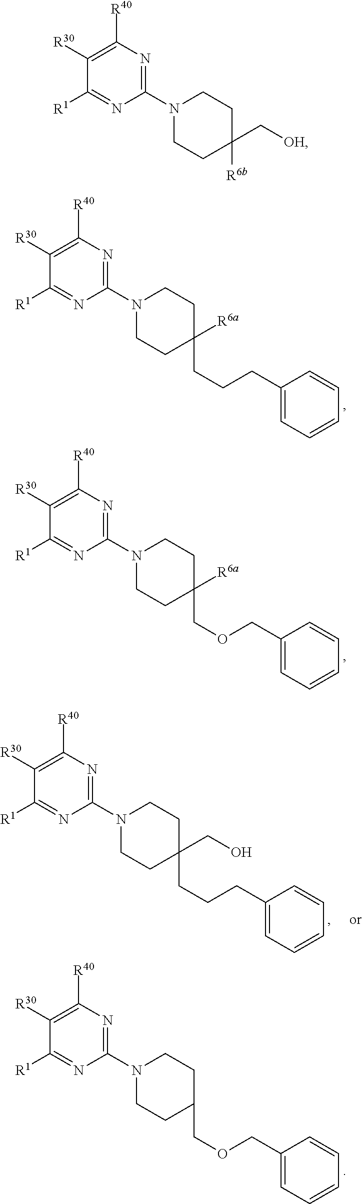

Aspect 65. The compound of Aspect 60, having a structure represented by a formula:

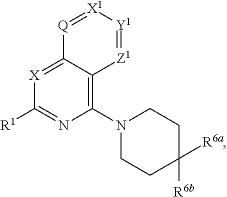

wherein Q is selected from N and CR²⁰; and wherein R²⁰ is selected from hydrogen, halogen, amino, hydroxy, cyano, nitro, —SF₅, C1-C3 alkyl, C1-C3 alkoxy, C1-C3 aminoalkyl, C1-C3 hydroxyalkyl, C1-C3 haloalkyl; wherein X¹ is selected from N and CR³¹; and wherein R³¹ is selected from hydrogen, halogen, amino, hydroxy, cyano, nitro, —SF₅, C1-C3 alkyl, C1-C3 alkoxy, C1-C3 aminoalkyl, C1-C3 hydroxyalkyl, C1-C3 haloalkyl; wherein Y¹ is selected from N and CR⁴¹; and wherein R⁴¹ is selected from hydrogen, halogen, amino, hydroxy, cyano, nitro, —SF₅, C1-C3 alkyl, C1-C3 alkoxy, C1-C3 aminoalkyl, C1-C3 hydroxyalkyl, C1-C3 haloalkyl; and wherein Z¹ is selected from N and CR⁵¹; and wherein R⁵¹ is selected from hydrogen, halogen, amino, hydroxy, cyano, nitro, —SF₅, C1-C3 alkyl, C1-C3 alkoxy, C1-C3 aminoalkyl, C1-C3 hydroxyalkyl, C1-C3 haloalkyl.

Aspect 66. The compound of Aspect 65, wherein Q is N; wherein X¹ is CR³¹; wherein Y¹ is CR⁴¹; and wherein Z¹ is CR⁵¹.

Aspect 67. The compound of Aspect 66, wherein each of X¹, Y¹, and Z¹ is CH.

Aspect 68. The compound of Aspect 65, wherein Q is CR²⁰; wherein X¹ is CR³¹; wherein Y¹ is CR⁴¹; and wherein Z¹ is CR⁵¹.

Aspect 69. The compound of Aspect 68, wherein each of Q, X¹, Y¹, and Z¹ is CH.

Aspect 70. The compound of Aspect 65, having a structure represented by a formula:

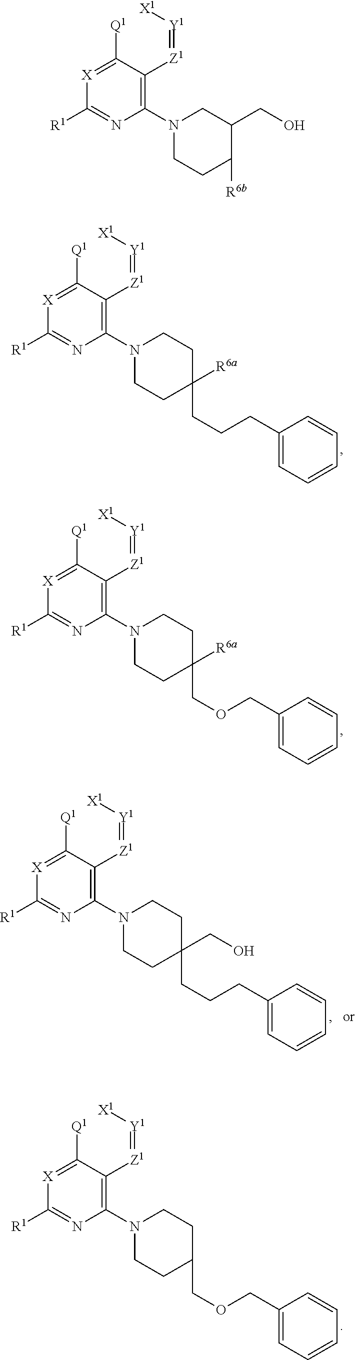

Aspect 71. The compound of Aspect 1, present as:

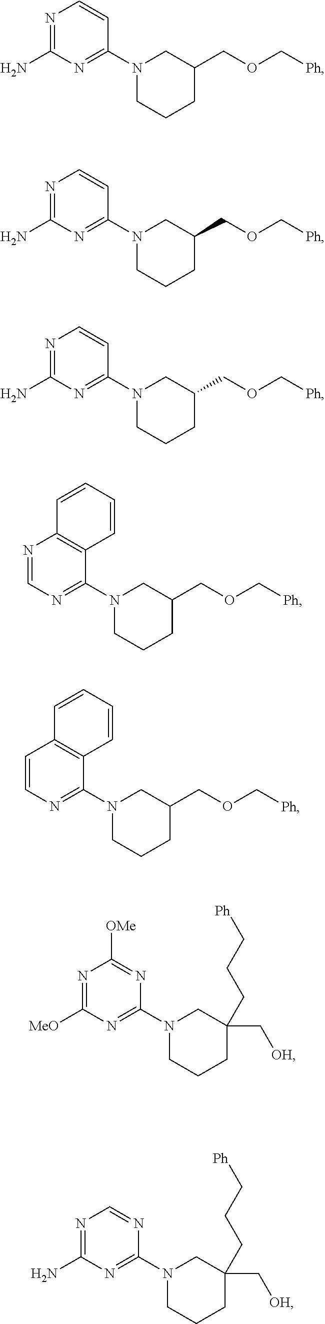

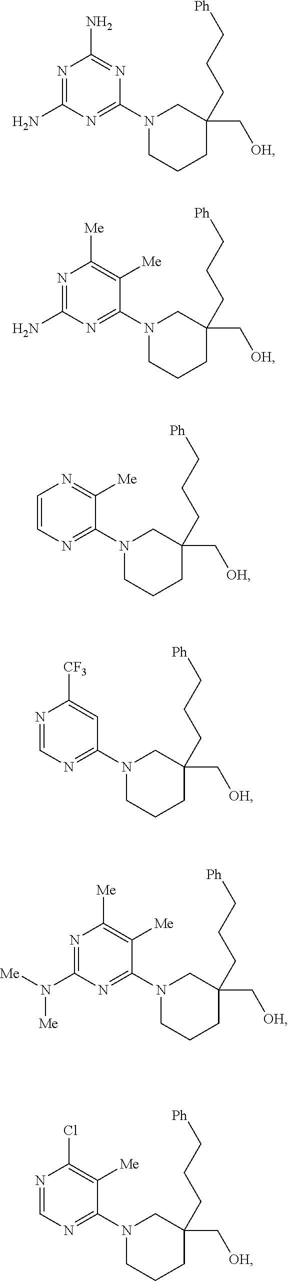 [SMILES: Nc1nc(N)nc(N2CCCC(CO)(CCCc3ccccc3)C2)n1 ; Cc1nc(N)nc(N2CCCC(CO)(CCCc3ccccc3)C2)c1C ; Cc1nccnc1N1CCCC(CO)(CCCc2ccccc2)C1 ; OCC1(CCCc2ccccc2)CCCN(c2cc(C(F)(F)F)ncn2)C1 ; Cc1nc(N(C)C)nc(N2CCCC(CO)(CCCc3ccccc3)C2)c1C ; Cc1c(Cl)ncnc1N1CCCC(CO)(CCCc2ccccc2)C1]

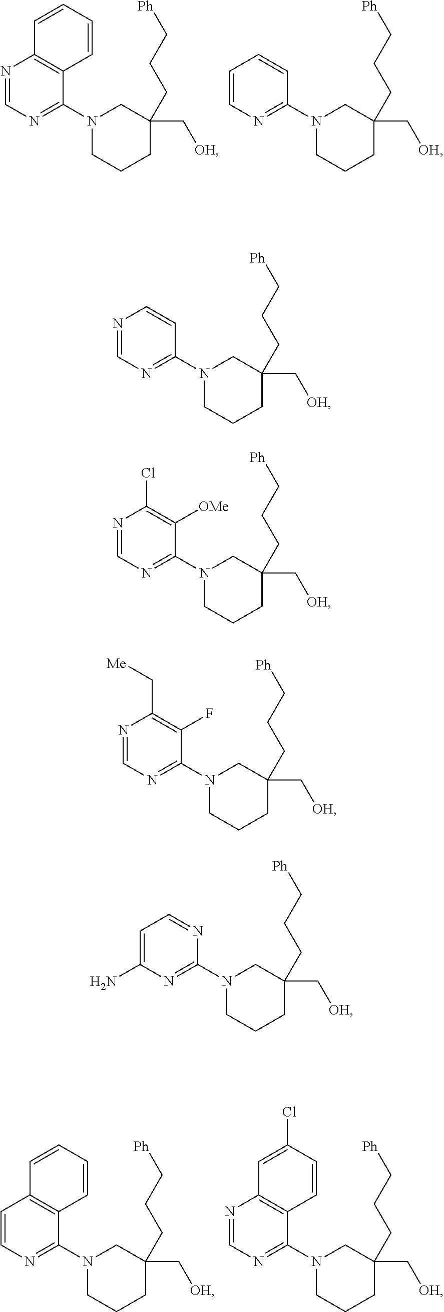

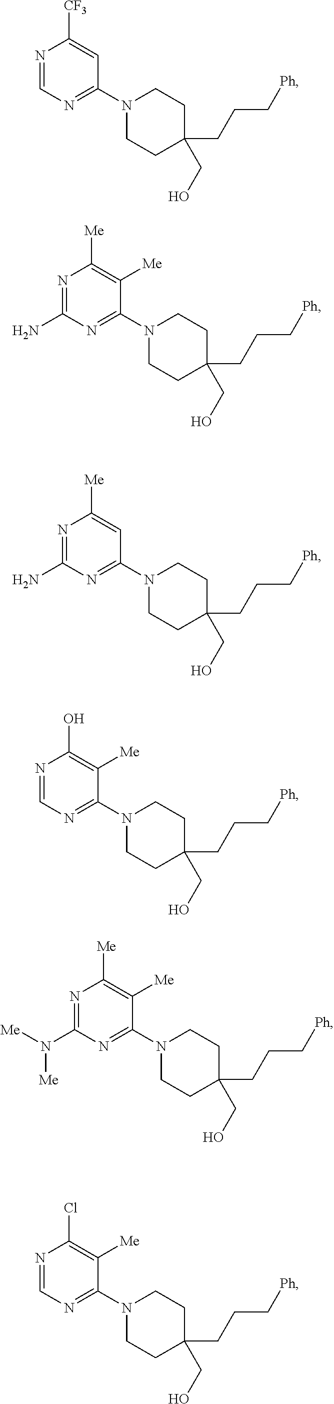 [SMILES: OCC1(CCCc2ccccc2)CCN(c2cc(C(F)(F)F)ncn2)CC1 ; Cc1nc(N)nc(N2CCC(CO)(CCCc3ccccc3)CC2)c1C ; Cc1cc(N2CCC(CO)(CCCc3ccccc3)CC2)nc(N)n1 ; Cc1c(O)ncnc1N1CCC(CO)(CCCc2ccccc2)CC1 ; Cc1nc(N(C)C)nc(N2CCC(CO)(CCCc3ccccc3)CC2)c1C ; Cc1c(Cl)ncnc1N1CCC(CO)(CCCc2ccccc2)CC1]

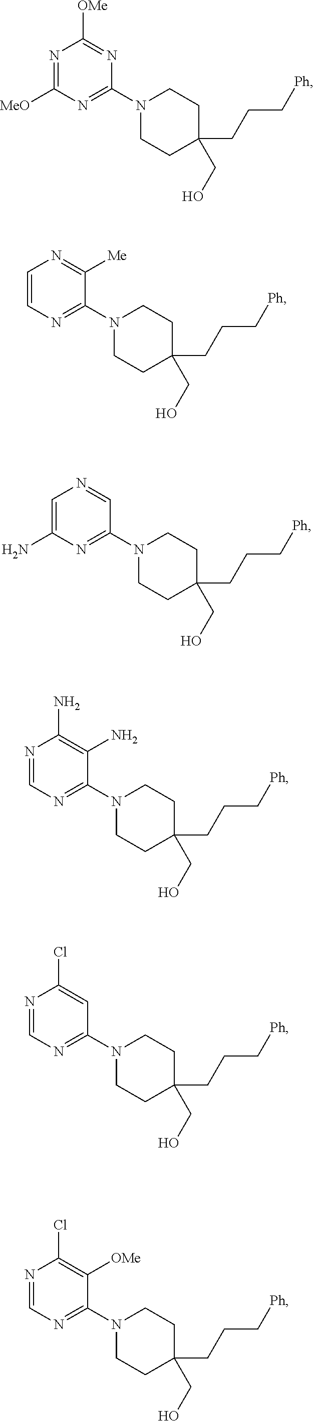 [SMILES: COc1nc(OC)nc(N2CCC(CO)(CCCc3ccccc3)CC2)n1 ; Cc1nccnc1N1CCC(CO)(CCCc2ccccc2)CC1 ; Nc1cncc(N2CCC(CO)(CCCc3ccccc3)CC2)n1 ; Nc1ncnc(N2CCC(CO)(CCCc3ccccc3)CC2)c1N ; OCC1(CCCc2ccccc2)CCN(c2cc(Cl)ncn2)CC1 ; COc1c(Cl)ncnc1N1CCC(CO)(CCCc2ccccc2)CC1]

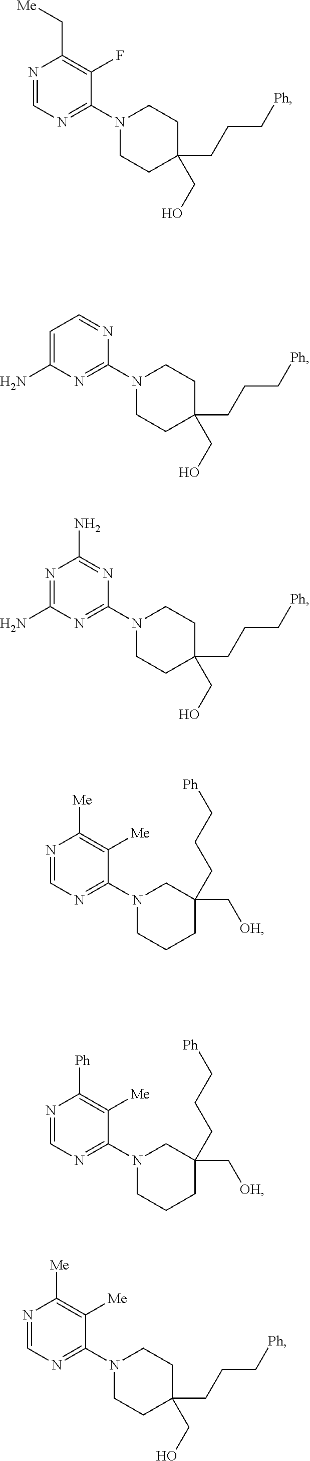 [SMILES: CCc1ncnc(N2CCC(CO)(CCCc3ccccc3)CC2)c1F ; Nc1ccnc(N2CCC(CO)(CCCc3ccccc3)CC2)n1 ; Nc1nc(N)nc(N2CCC(CO)(CCCc3ccccc3)CC2)n1 ; Cc1ncnc(N2CCCC(CO)(CCCc3ccccc3)C2)c1C ; Cc1c(-c2ccccc2)ncnc1N1CCCC(CO)(CCCc2ccccc2)C1 ; Cc1ncnc(N2CCC(CO)(CCCc3ccccc3)CC2)c1C]

or a subgroup thereof.

Aspect 72. A pharmaceutical composition comprising a therapeutically effective amount of a compound of any of Aspects 1-71, or a pharmaceutically acceptable salt, solvate, or polymorph thereof, and a pharmaceutically acceptable carrier.

Aspect 73. The pharmaceutical composition of Aspect 72, further comprising at least one agent known to treat a cancer.

Aspect 74. The pharmaceutical composition of Aspect 73, wherein the at least one agent known to treat a cancer is a hormone therapy agent; an alkylating agent, an antimetabolite agent, an antineoplastic antibiotic agent, a mitotic inhibitor agent, a mTor inhibitor agent, other chemotherapeutic agent, or combinations thereof.

Aspect 75. The pharmaceutical composition of Aspect 74, wherein the at least one agent known to treat a cancer is a hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt thereof.

Aspect 76. The pharmaceutical composition of Aspect 74, wherein the at least one agent known to treat a cancer is a antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

Aspect 77. The pharmaceutical composition of Aspect 74, wherein the at least one agent known to treat a cancer is an antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

Aspect 78. The pharmaceutical composition of Aspect 74, wherein the at least one agent known to treat a cancer is an alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt.

Aspect 79. The pharmaceutical composition of Aspect 74, wherein the at least one agent known to treat a cancer is a mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt.

Aspect 80. The pharmaceutical composition of Aspect 74, wherein the at least one agent known to treat a cancer is a mTor inhibitor agent is selected from one or more of the group consisting of everolimus, siroliumus, and temsirolimus, or a pharmaceutically acceptable salt thereof.

Aspect 81. wherein the at least one agent known to treat a cancer is selected from uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin (Eloxatin®), iressa (gefinitib, Zd1839), XELODA® (capecitabine), Tarceva® (erlotinib), azacitidine (5-Azacytidine; 5-AzaC), temozolomide (Temodar®), gemcitabine (e.g., GEMZAR® (gemcitabine HCl)), vasostatin, and combinations thereof.

Aspect 82. The pharmaceutical composition of any one of Aspects 1-81, wherein the at least one compound and the at least one agent known to treat a cancer are co-formulated.

Aspect 83. A method for the treatment of a cancer associated with an AVIL dysfunction in a mammal comprising the step of administering to the mammal a therapeutically effective amount of at least one compound of any one of Aspects 1-71, or a pharmaceutical composition of any one of Aspects 1-82.

Aspect 84. The method of Aspect 83, wherein the compound or the pharmaceutical composition modulates the activity or expression of AVIL.

Aspect 85. The method of Aspect 84, wherein the compound or the pharmaceutical composition inhibits AVIL activity.

Aspect 86. The method of Aspect 84, wherein the compound or the pharmaceutical composition decreases expression of AVIL.

Aspect 87. The method of any one of Aspects 83-86, wherein the mammal is a human.

Aspect 88. The method of any one of Aspects 83-87, wherein the mammal has been diagnosed with a need for treatment of the disorder prior to the administering step.

Aspect 89. The method of any one of Aspects 83-88, further comprising the step of identifying a mammal in need of treatment of the disorder.

Aspect 90. The method of any one of Aspects 83-89, wherein the cancer is selected from the cancer is selected from brain cancer and cancerous tumors such as glioblastomas, rhabdosarcomas, gliomas, lung cancer, bladder cancer including bladder urothelial carcinoma, and renal cancer including kidney clear cell carcinoma.

Aspect 91. A method for the treatment of a clinical condition associated with an AVIL dysfunction in a mammal comprising the step of administering to the mammal a therapeutically effective amount of at least one compound of any one of Aspects 1-71, or a pharmaceutical composition of any one of Aspects 1-82.

Aspect 92. The method of Aspect 91, wherein the mammal is a human.

Aspect 93. The method of Aspect 91, wherein the mammal has been diagnosed with a need for modulating AVIL activity prior to the administering step.

Aspect 94. The method of Aspect 91, further comprising the step of identifying a mammal in need of modulating AVIL activity.

Aspect 95. The method of any one of Aspects 91-94, wherein the mammal has been diagnosed with a need for treating a cancer prior to the administering step.

Aspect 96. The method of any one of Aspects 91-94, further comprising diagnosing the mammal with a need for treating a cancer prior to the administering step.

Aspect 97. The method of Aspect 95 or 96, wherein the cancer is selected from the cancer is selected from brain cancer and cancerous tumors such as glioblastomas, rhabdosarcomas, gliomas, lung cancer, bladder cancer including bladder urothelial carcinoma, and renal cancer including kidney clear cell carcinoma.

Aspect 98. A method for modulating of AVIL activity in at least one cell, comprising the step of contacting the at least one cell with an effective amount of at least one compound of any one of Aspects 1-71, or a pharmaceutical composition of any one of Aspects 1-82.

Aspect 99. The method of Aspect 98, wherein the cell is mammalian.

Aspect 100. The method of Aspect 98, wherein the cell is human.

Aspect 101. The method of any one of Aspects 98-100, wherein the cell has been isolated from a mammal prior to the contacting step.

Aspect 102. The method of any one of Aspects 98-101, wherein contacting is via administration of the compound or the pharmaceutical composition to a mammal.

Aspect 103. The method of any one of Aspects 98-102, wherein the mammal has been diagnosed with a need a need for modulating AVIL activity prior to the administering step.

Aspect 104. The method of any one of Aspects 98-102, wherein the mammal has been diagnosed with a need for treatment of a clinical condition related to AVIL activity prior to the administering step.

Aspect 105. The method of any one of Aspects 98-104, wherein the mammal has been diagnosed with a need for treating a cancer prior to the administering step.

Aspect 106. The method of any one of Aspects 98-104, further comprising diagnosing the mammal with a need for treating a cancer prior to the administering step.

Aspect 107. The method of Aspect 105 or 106, wherein the cancer is selected from the cancer is selected from brain cancer and cancerous tumors such as glioblastomas, rhabdosarcomas, gliomas, lung cancer, bladder cancer including bladder urothelial carcinoma, and renal cancer including kidney clear cell carcinoma.

Aspect 108. The method of any one of Aspects 83-107, wherein the compound exhibits inhibition of AVIL activity or expression with an IC50 of less than about 100 μM when determined in a cell-based proliferation assay using a U87 cell line.

Aspect 109. The method of Aspect 108, wherein the IC50 is less than about 75 μM.

Aspect 110. The method of Aspect 108, wherein the IC50 is less than about 50 μM.

Aspect 111. The method of Aspect 108, wherein the IC50 is less than about 10 μM.

Aspect 112. The method of Aspect 108, wherein the IC50 is less than about 1 μM.

Aspect 113. The method of any one of Aspects 83-112, wherein the compound exhibits inhibition of AVIL activity or expression with an IC50 of greater than about 100 μM on non-target cells having normal AVIL function when determined in an in vitro cell-based proliferation assay.

Aspect 114. The method of Aspect 113, wherein the non-target cells having normal AVIL function are primary astrocyte cells.

Aspect 115. The method of Aspects 113 or 114, wherein the IC50 is greater than about 250 μM.

Aspect 116. The method of Aspects 113 or 114, wherein the IC50 is greater than about 500 μM.

Aspect 117. The method of Aspects 113 or 114, wherein the IC50 is greater than about 1 mM.

Aspect 118. A kit comprising at least one compound of any one of Aspects 1-71, or a pharmaceutical composition of any one of Aspects 1-82; and one or more of: a) at least one agent known to decrease AVIL activity; b) at least one agent known to treat a cancer; c) instructions for treating a cancer; d) instructions for treating a clinical condition associated with an AVIL dysfunction.

Aspect 119. The kit of Aspect 118, wherein the at least one compound or the at least one product and the at least one agent are co-formulated.

Aspect 120. The kit of Aspect 118, wherein the at least one compound or the at least one product and the at least one agent are co-packaged.

Aspect 121. The kit of any one of Aspects 118-120, wherein the at least one agent known to treat a cancer is a hormone therapy agent; an alkylating agent, an antimetabolite agent, an antineoplastic antibiotic agent, a mitotic inhibitor agent, a mTor inhibitor agent, other chemotherapeutic agent, or combinations thereof.

Aspect 122. The kit of Aspect 121, wherein the at least one agent known to treat a cancer is a hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt thereof.

Aspect 123. The kit of Aspect 121, wherein the at least one agent known to treat a cancer is a antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

Aspect 124. The kit of Aspect 121, wherein the at least one agent known to treat a cancer is an antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

Aspect 125. The kit of Aspect 121, wherein the at least one agent known to treat a cancer is an alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt.

Aspect 126. The kit of Aspect 121, wherein the at least one agent known to treat a cancer is a mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt.

Aspect 127. The kit of Aspect 121, wherein the at least one agent known to treat a cancer is a mTor inhibitor agent is selected from one or more of the group consisting of everolimus, siroliumus, and temsirolimus, or a pharmaceutically acceptable salt thereof.

Aspect 128. The kit of Aspect 121, wherein the at least one agent known to treat a cancer is selected from uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin (Eloxatin®), iressa (gefinitib, Zd1839), XELODA® (capecitabine), Tarceva® (erlotinib), azacitidine (5-Azacytidine; 5-AzaC), temozolomide (Temodar®), gemcitabine (e.g., GEMZAR® (gemcitabine HCl)), vasostatin, and combinations thereof.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The various testing methods and procedures referenced in the examples described herein are discussed in more detail below.

### Example 1. Compound IC₅₀ Values in Glioblastoma Cells

IC₅₀ in glioblastoma vs control: Glioblastoma cell line U87 cells were cultured in Minimum Essential Medium Eagle (MEM), supplemented with 1 mM sodium pyruvate, 1% nonessential amino acids, 0.15% sodium bicarbonate, and 10% fetal bovine serum (FBS); Immortalized human astrocytes (non-cancer control) were grown in DMEM/F12 with 4.5 g/L of glucose, supplemented with 10% FBS. Different concentration of compounds were added to the cells. Three days later, a MTT assay (Sigma) was performed to monitor cell viability.

In this example, samples of glioblastoma cells (U87) and astrocyte cells (non-cancer control) were exposed various disclosed compounds to determine the effectiveness of the compounds for inhibiting cell growth.

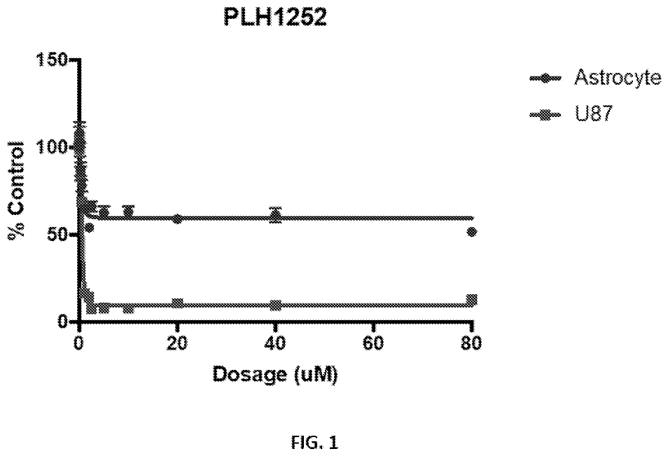

FIG. 1 shows cell survival curves comparing the effect of compound PLH1252 on U87 glioblastoma cells vs. astrocyte cells (non-cancer control) at varied doses. The results demonstrate that most if not all of the U87 cells were wiped out with less than 1 μM (e.g., 500 nm) of compound PLH1252, whereas no significant effect was seen in astrocytes.

The IC₅₀ for different test compounds were measured using Prims 7 IC₅₀ software, and is reported in the Table 1, below. The results show that the disclosed compounds are effective in inhibiting cell growth.

TABLE 1

| | Molecular | | | |
|---|---|---|---|---|
| | Weight | Compound | IC₅₀ | IC₅₀ |
| Chemical: | (g/mol) | designation | (U87) | (astrocyte) |
| 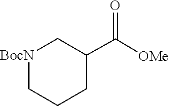 [SMILES: COC(=O)C1CCCN(C(=O)OC(C)(C)C)C1] | 243.30 | PLH1190 | >80 μM | >80 μM |
| 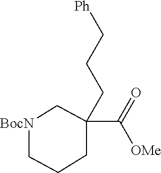 [SMILES: COC(=O)C1(CCCc2ccccc2)CCCN(C(=O)OC(C)(C)C)C1] | 361.48 | PLH1195 | ~22.5 μM | 65 μM |
| 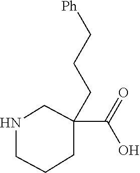 [SMILES: O=C(O)C1(CCCc2ccccc2)CCCNC1] | 261.37 | PLH1197 | ~80 μM | >80 μM |
| 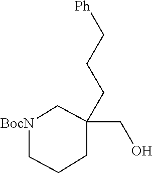 [SMILES: CC(C)(C)OC(=O)N1CCCC(CO)(CCCc2ccccc2)C1] | 333.47 | PLH1196 | ~30 μM | >80 μM |
| 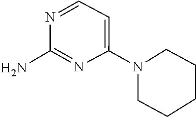 [SMILES: Nc1nccc(N2CCCCC2)n1] | 178.24 | JDDUVA12 | >80 μM | >80 μM |
| 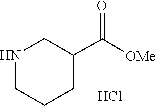 | 179.64 | PLH1155 | >80 μM | >80 μM |
| 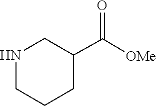 | 143.19 | JDDUVA02 | >80 μM | >80 μM |
| 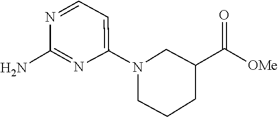 [SMILES: COC(=O)C1CCCN(c2ccnc(N)n2)C1] | 236.28 | JDDUVA16 | >80 μM | — |
| 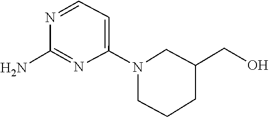 [SMILES: Nc1nccc(N2CCCC(CO)C2)n1] | 208.27 | JDDUVA18 | >80 μM | — |
| 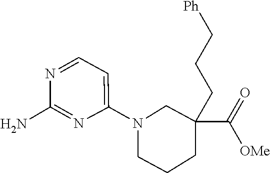 [SMILES: COC(=O)C1(CCCc2ccccc2)CCCN(c2ccnc(N)n2)C1] | 354.45 | JDDUVA20 | ~10.9 μM | 70 μM |
| 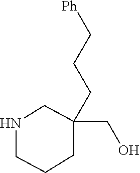 [SMILES: OCC1(CCCc2ccccc2)CCCNC1] | 233.36 | PLH1211 | >80 μM | >80 μM |
| 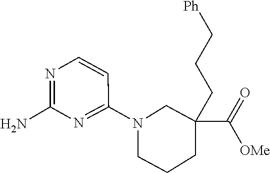 [SMILES: COC(=O)C1(CCCc2ccccc2)CCCN(c2ccnc(N)n2)C1] | 354.45 | PLH1209 | 6 μM | 76 μM |
| 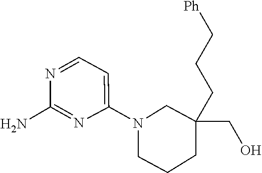 [SMILES: Nc1nccc(N2CCCC(CO)(CCCc3ccccc3)C2)n1] | 326.44 | PLH1215 | ~11.7 μM | >80 μM |
| 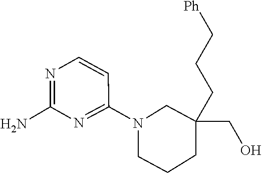 [SMILES: Nc1nccc(N2CCCC(CO)(CCCc3ccccc3)C2)n1] | 326.44 | JDDUVA28 | ~12.2 μM | >80 μM |
| JDDUVA28-P1 | | | | |
| 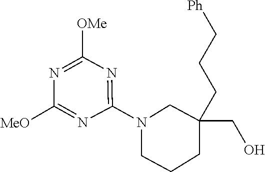 [SMILES: COc1nc(OC)nc(N2CCCC(CO)(CCCc3ccccc3)C2)n1] | 372.47 | PLH1230 | 63.6 μM | >80 μM |
| 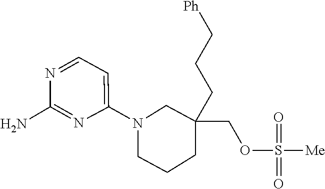 [SMILES: CS(=O)(=O)OCC1(CCCc2ccccc2)CCCN(c2ccnc(N)n2)C1] | 404.53 | PLH1224 | ~50 μM | >80 μM |
| 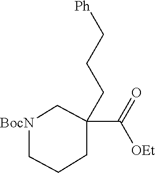 [SMILES: CCOC(=O)C1(CCCc2ccccc2)CCCN(C(=O)OC(C)(C)C)C1] | 375.51 | PLH1225 | ~15 μM | >80 μM |
| 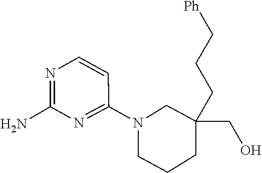 [SMILES: Nc1nccc(N2CCCC(CO)(CCCc3ccccc3)C2)n1] | 326.44 | JDDUVA38 | ~11 μM | 56 μM |
| 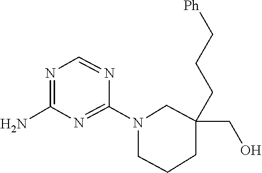 [SMILES: Nc1ncnc(N2CCCC(CO)(CCCc3ccccc3)C2)n1] | 327.43 | PLH1239 | >80 μM | — |
| 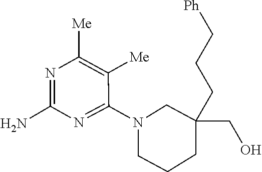 [SMILES: Cc1nc(N)nc(N2CCCC(CO)(CCCc3ccccc3)C2)c1C] | 354.50 | PLH1252 | ~1 μM ~600 nM | 45 μM ~80 μM |
| 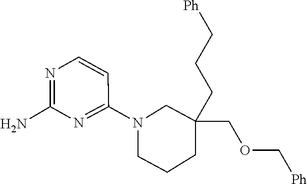 [SMILES: Nc1nccc(N2CCCC(CCCc3ccccc3)(COCc3ccccc3)C2)n1] | 416.57 | PLH1253 | ~1.7 μM ~3.4 μM | ~30 μM ~12.6 μM |
| 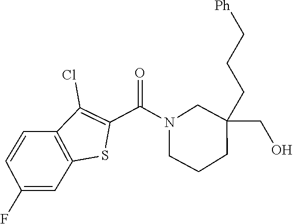 [SMILES: O=C(c1sc2cc(F)ccc2c1Cl)N1CCCC(CO)(CCCc2ccccc2)C1] | 445.98 | PLH1254 | 16 μM | 49 μM |
| 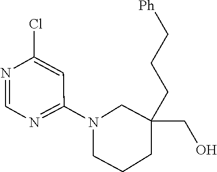 [SMILES: OCC1(CCCc2ccccc2)CCCN(c2cc(Cl)ncn2)C1] | 379.43 | PLH1259 | 2.5 μM 8 μM | 10.2 μM 80 μM |
| 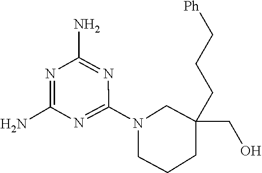 [SMILES: Nc1nc(N)nc(N2CCCC(CO)(CCCc3ccccc3)C2)n1] | 342.45 | PLH1258 | 3.6 μM | >80 μM |
| 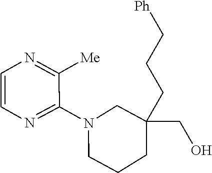 [SMILES: Cc1nccnc1N1CCCC(CO)(CCCc2ccccc2)C1] | 325.46 | PLH1234 | 20 μM | 51.8 μM |
| 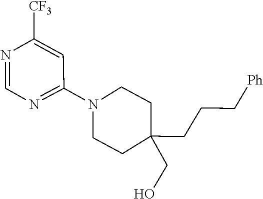 [SMILES: OCC1(CCCc2ccccc2)CCN(c2cc(C(F)(F)F)ncn2)CC1] | 379.43 | JDDUVA47 | 13 μM | >80 μM |
| 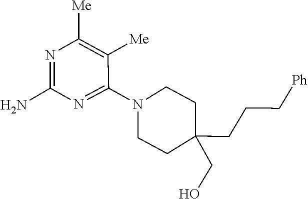 [SMILES: Cc1nc(N)nc(N2CCC(CO)(CCCc3ccccc3)CC2)c1C] | 354.50 | JDDUVA43 | 1 μM 2.5 μM | 47 μM 60 μM |
| 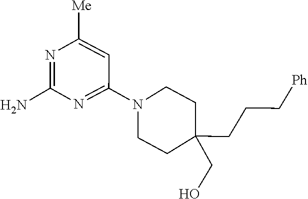 [SMILES: Cc1cc(N2CCC(CO)(CCCc3ccccc3)CC2)nc(N)n1] | 340.47 | JDDUVA44 | 1 μM 2.5 μM | 10 μM 20 μM |
| 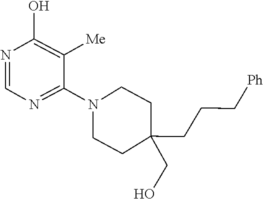 [SMILES: Cc1c(O)ncnc1N1CCC(CO)(CCCc2ccccc2)CC1] | 341.46 | JDDUVA48 | 14.1 μM | 7.32 μM |
| 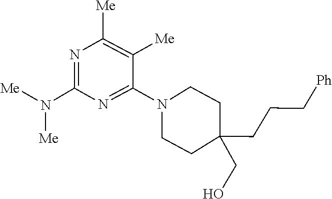 [SMILES: Cc1nc(N(C)C)nc(N2CCC(CO)(CCCc3ccccc3)CC2)c1C] | 382.55 | JDDUVA44 | 4.03 μM | 7.21 μM |
| 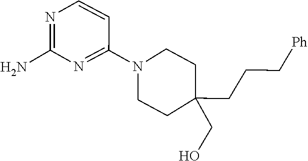 [SMILES: Nc1nccc(N2CCC(CO)(CCCc3ccccc3)CC2)n1] | 326.44 | JDDUVA49 | 3 μM | >80 μM |
| 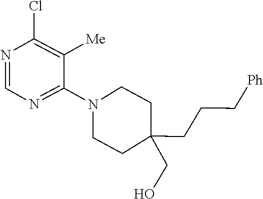 [SMILES: Cc1c(Cl)ncnc1N1CCC(CO)(CCCc2ccccc2)CC1] | 359.90 | JDDUVA50 | 21 μM | >80 μM |
| 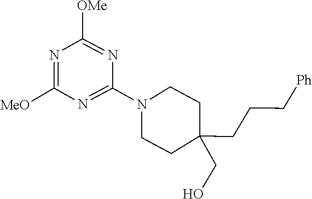 [SMILES: COc1nc(OC)nc(N2CCC(CO)(CCCc3ccccc3)CC2)n1] | 372.47 | JDDUVA52 | 41 μM | >80 μM |
| 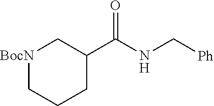 [SMILES: CC(C)(C)OC(=O)N1CCCC(C(=O)NCc2ccccc2)C1] | 318.42 | PLH1273 | >80 μM | >80 μM |
| 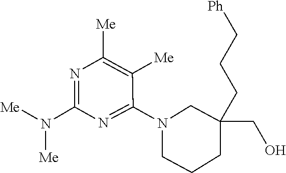 [SMILES: Cc1nc(N(C)C)nc(N2CCCC(CO)(CCCc3ccccc3)C2)c1C] | 382.55 | PLH1276 | 19 μM | >80 μM |
| 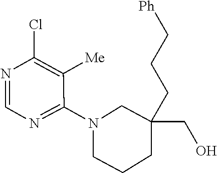 [SMILES: Cc1c(Cl)ncnc1N1CCCC(CO)(CCCc2ccccc2)C1] | 359.90 | PLH1279 | 8 μM | >80 μM (no effect) |
| 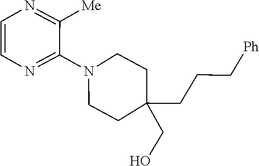 [SMILES: Cc1nccnc1N1CCC(CO)(CCCc2ccccc2)CC1] | 325.46 | JDDUVA53 | 30 μM | >80 μM |
| 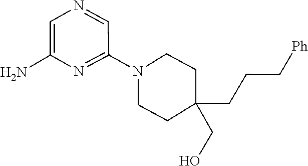 [SMILES: Nc1cncc(N2CCC(CO)(CCCc3ccccc3)CC2)n1] | 326.44 | JDDUVA55 | 16 μM | >80 μM |
| 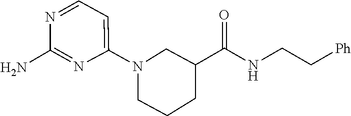 [SMILES: Nc1nccc(N2CCCC(C(=O)NCCc3ccccc3)C2)n1] | 325.42 | PLH1285 | 17 μM | >80 μM (no effect) |
| 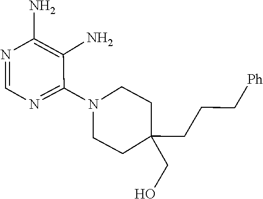 [SMILES: Nc1ncnc(N2CCC(CO)(CCCc3ccccc3)CC2)c1N] | 341.46 | JDDUVA51 | 12 μM | >80 μM |
| 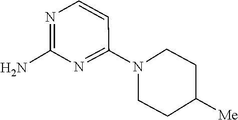 [SMILES: CC1CCN(c2ccnc(N)n2)CC1] | 192.27 | JDDUVA59 | 72 μM | >80 μM (no effect) |
| 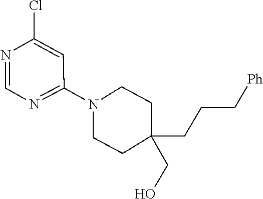 [SMILES: OCC1(CCCc2ccccc2)CCN(c2cc(Cl)ncn2)CC1] | 345.87 | JDDUVA60 | <1 μM 12 μM | >80 μM >80 μM |
| 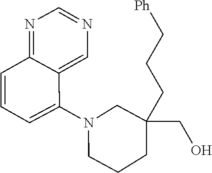 [SMILES: OCC1(CCCc2ccccc2)CCCN(c2cccc3ncncc23)C1] | 361.49 | PLH1295 | <1 μM 500 nM | 22 μM 30 μM |
| 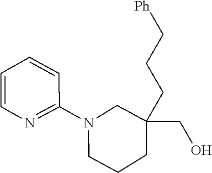 [SMILES: OCC1(CCCc2ccccc2)CCCN(c2ccccn2)C1] | 310.44 | PLH1291 | 14 μM | >80 μM |
| 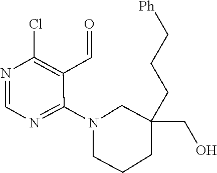 [SMILES: O=Cc1c(Cl)ncnc1N1CCCC(CO)(CCCc2ccccc2)C1] | 373.88 | PLH1293 | 8 μM | 18 μM |
| 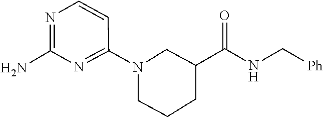 [SMILES: Nc1nccc(N2CCCC(C(=O)NCc3ccccc3)C2)n1] | 311.39 | PLH1290 | >80 μM | >80 μM |
| 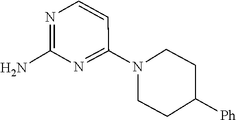 [SMILES: Nc1nccc(N2CCC(c3ccccc3)CC2)n1] | 254.34 | JDDUVA63 | 16 μM | >80 μM |
| 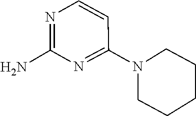 [SMILES: Nc1nccc(N2CCCCC2)n1] | 178.24 | JDDUVA12 | | >80 μM |
| 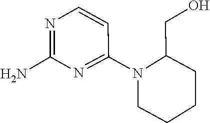 [SMILES: Nc1nccc(N2CCCCC2CO)n1] | 208.27 | JDDUVA61 | >80 μM | >80 μM (no effect) |
| 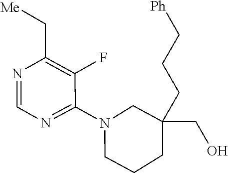 [SMILES: CCc1ncnc(N2CCCC(CO)(CCCc3ccccc3)C2)c1F] | 357.47 | PLH2002 | 1.5 μM | 1.1 μM |
| 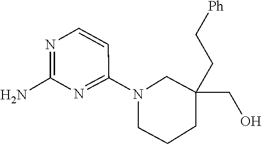 [SMILES: Nc1nccc(N2CCCC(CO)(CCc3ccccc3)C2)n1] | 312.42 | PLH1304 | 3.83 μM | ~80 μM |
| 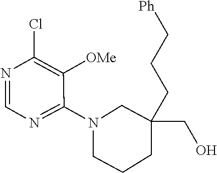 [SMILES: COc1c(Cl)ncnc1N1CCCC(CO)(CCCc2ccccc2)C1] | 375.90 | PLH1302 | ~2.5 μM 2.3 μM | ~20 μM 10 μM |
| 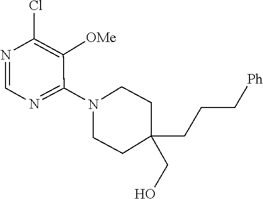 [SMILES: COc1c(Cl)ncnc1N1CCC(CO)(CCCc2ccccc2)CC1] | 375.90 | JDDUVA67 | ~2.5 μM | ~6 μM |
| 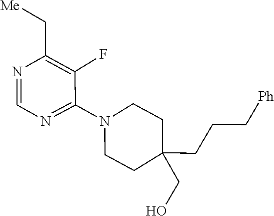 [SMILES: CCc1ncnc(N2CCC(CO)(CCCc3ccccc3)CC2)c1F] | 357.47 | JDDUVA68 | 3.6 μM | 12.5 μM |
| 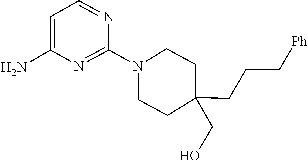 [SMILES: Nc1ccnc(N2CCC(CO)(CCCc3ccccc3)CC2)n1] | 326.44 | JDDUVA69 | 13.6 μM | >80 μM (no effect) |
| 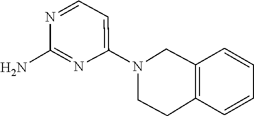 [SMILES: Nc1nccc(N2CCc3ccccc3C2)n1] | 226.28 | JDDUVA71 | >80 μM (no effect) | >80 μM (no effect) |
| 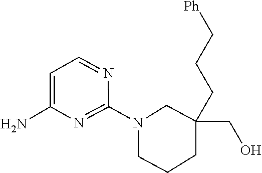 [SMILES: Nc1ccnc(N2CCCC(CO)(CCCc3ccccc3)C2)n1] | 326.44 | PLH2006 | >80 μM (no effect) | >80 μM (no effect) |
| 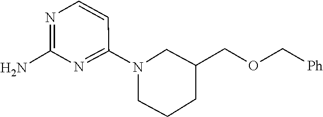 [SMILES: Nc1nccc(N2CCCC(COCc3ccccc3)C2)n1] | 298.39 | PLH2027 | ~50 μM | ~80 μM |
| 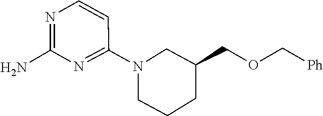 [SMILES: Nc1nccc(N2CCC[C@H](COCc3ccccc3)C2)n1] | 298.39 | PLH2032 | 4.3 μM | ~40 μM |
| 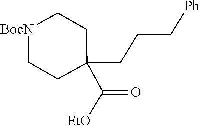 [SMILES: CCOC(=O)C1(CCCc2ccccc2)CCN(C(=O)OC(C)(C)C)CC1] | 375.51 | JDDUVA76 | ~30 μM | ~60 μM |
| 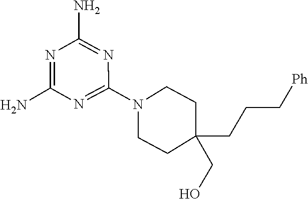 [SMILES: Nc1nc(N)nc(N2CCC(CO)(CCCc3ccccc3)CC2)n1] | 342.45 | JDDUVA78 | 12 μM | ~40 μM |
| 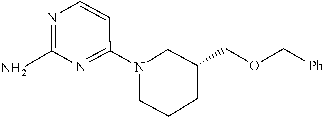 [SMILES: Nc1nccc(N2CCC[C@@H](COCc3ccccc3)C2)n1] | 298.39 | PLH2040 | 2.8 μM | >80 μM |
| 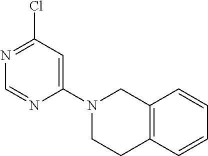 [SMILES: Clc1cc(N2CCc3ccccc3C2)ncn1] | 245.71 | JDDUVA81 | >80 μM | >80 μM |
| 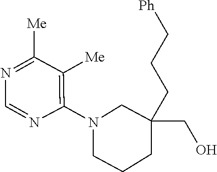 [SMILES: Cc1ncnc(N2CCCC(CO)(CCCc3ccccc3)C2)c1C] | 339.48 | PLH2056 | <2.5 μM | ~80 μM |
| 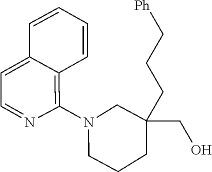 [SMILES: OCC1(CCCc2ccccc2)CCCN(c2nccc3ccccc23)C1] | 360.50 | PLH2058 | 70 μM | 80 μM |
| 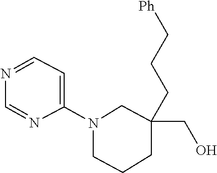 [SMILES: OCC1(CCCc2ccccc2)CCCN(c2ccncn2)C1] | 311.43 | PLH2062 | ~30 μM | >80 μM |
| 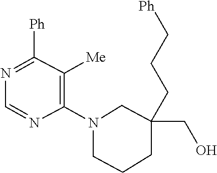 [SMILES: Cc1c(-c2ccccc2)ncnc1N1CCCC(CO)(CCCc2ccccc2)C1] | 401.55 | PLH2063 | ~80 μM | ~80 μM |
| 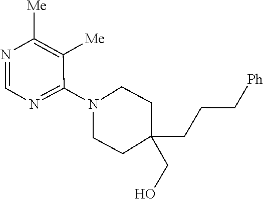 [SMILES: Cc1ncnc(N2CCC(CO)(CCCc3ccccc3)CC2)c1C] | 339.48 | JDDUVA96 | ~4 μM | ~8 μM |
| 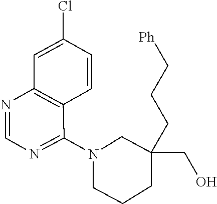 [SMILES: OCC1(CCCc2ccccc2)CCCN(c2ncnc3cc(Cl)ccc23)C1] | 395.93 | PLH2074 | ~2 μM | ~15 μM |

### Example 2: Immunoprecipitation of Protein

293T cells were first transfected with MYC tagged AVIL as follows: transfect pQCXIH-myc-AVIL plasmid in 6 well plate of 293T for 48 hrs. 200 ul opti-medium+7 ul GeneCellin (Polyplus transfection Co.)+4 ug plasmids in each well. Transfected AVIL cells were treated with the IC50 dose of the test compounds for 24 hrs. Protein extract was then prepared on ice by adding 1 ml IP lysis buffer+protease inhibitor+cocktail+PMSF, and let stand on ice for 30 min. Cells were then scraped into EP tubes and sonicated continuously for 5 sec with power setting 3. Tubes were then centrifuged in 4° C. at 13000 rpm for 20 min, and the upper aqueous phase was then transferred to pre-chilled tubes. Protein concentration was then determined using Bio-Rad protein assay. Preclear cell extracts were treated with 50 ul ProteinG/Sepharose per my lysate for 1 hr. Then 500 ug precleared cell extracts were taken and adjusted to 500 ul with lysis buffer for 2 ug myc antibody or IgG control and incubated at 4° C. overnight. Then, 25 ul ProteinG/Sepharose beads were added and incubated at 4° C. overnight. Beads were then washed four times with 1 ml IP wash buffer for 3 min, and spun at 1000 rpm to pellet beads. All wash buffer was removed with pipette, and 20 ul 2×SDS loading buffer was added and then boiled at 95° C. for 10 min to denature. This was centrifuged at 12000 rpm for 30 sec., and the supernatant was then transferred to a fresh tube and load for SDS-PAGE.

Western blot was performed with R-actin antibody. To measure protein levels, cell lysates were resolved by denaturing gel electrophoresis, before electrotransfer to a Protran nitrocellulose membrane. The membrane was subjected to western blot analysis with antibodies against the proteins of interest. The following antibodies and dilutions were used: rabbit anti-AVIL (1:2000; Sigma SAB2100191).

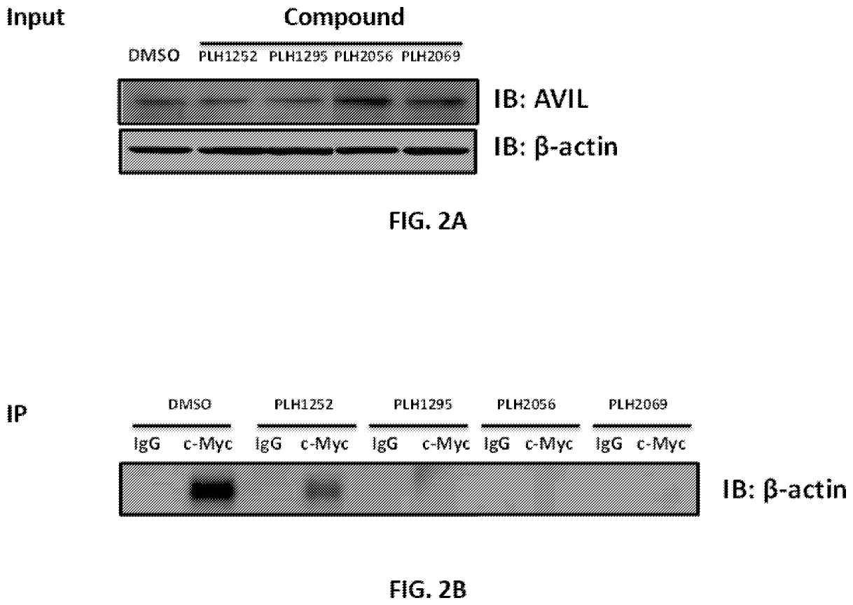

Results of immunoprecipitation and western blot are shown in FIGS. 2A and 2B.

### Example 3: LIN28B Gene Expression

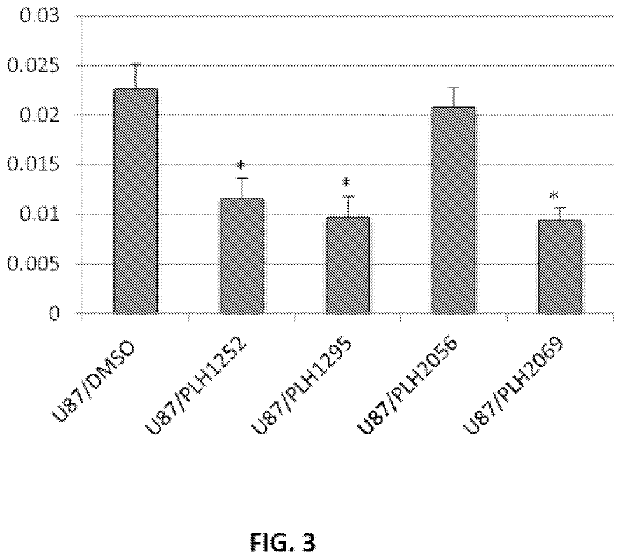

U87 cells were treated at the IC₅₀ concentration of the five test compounds. Cells were harvested. RNA was extracted using TRIzol reagent (Invitrogen) and quantified with Nanodrop (Thermo). cDNA was generated by AMV-RT kit (NEB), and a random hexamer primer. Real-time qPCR was carried out on the StepOne Plus system from Applied Biosystems using SYBR mix (Thermo). LIN28B expression was measured by qRT-PCR and is represented relative to GAPDH levels. FIG. 3 shows down-regulation of LIN28B by test compounds PLH1252, PLH1295, PLH2065, and PLH2069. The following primers were used for AVIL and GAPDH amplifications:

GAPDH-S,
5′-ATCACTGCCACCCAGAAGAC-3′,
GAPDH-AS,
5′-AAGGCCATGCCAGTGAGCTT-3′,
AVIL-S,
5′-TGCGAACTCAGGCAGAGCACTA-3′,
AVIL-AS,
5′-CAGTGTTCTCGCTGCCATCACA-3′

### Example 4: Synthesis of Aryl Chlorides

### 4-Chloro-1,3,5-triazin-2-amine

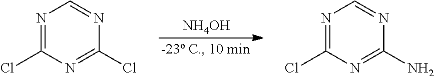 [SMILES: Clc1ncnc(Cl)n1 ; "NH4OH" ; Nc1ncnc(Cl)n1]

2,4-Dichloro-1,3,5-triazine (299.9 mg, 2 mmol) was dissolved in ammonium hydroxide (6 mL) cooled to −23° C. After 10 min precipitate was collected by vacuum filtration, washing with cold water. Dried in vacuo and used without further purification.

### 6-Chloro-1,3,5-triazine-2,4-diamine

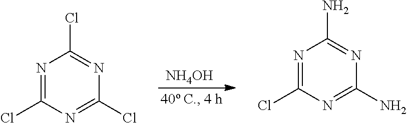 [SMILES: Clc1nc(Cl)nc(Cl)n1 ; "NH4OH" ; Nc1nc(N)nc(Cl)n1]

Cycanuric chloride (739.3 mg, 4.01 mmol) was suspended in ammonium hydroxide (10 mL) and warmed to 40° C. After 4 hr, the mixture was diluted with water (˜20 mL) and filtered, washing with water (2×20 mL), and dried in vacuo to give white crystals (531.8 mg, 91%) which was used without further purification.

### 4-Chloropyrimidine

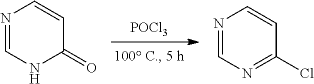

4(3H)-Pyrimidinone (401.2 mg, 4.18 mmol) and POCl₃ (4 mL) were charged to a pressure flask. Flask was flushed with nitrogen and heated to 100° C. for 5 h. Yellow precipitate was filtered and washed with 20 mL of hexane. Yellow-orange precipitate was dried in vacuo and used without further purification (332.3 mg, 70%).

### Example 5: Synthesis of Amine Cores

Reduction Procedure: Ester or carboxylic acid was dissolved in THF in a 2-neck r.b. flask under nitrogen atmosphere. The solution was cooled to −78° C. Lithium aluminum hydride (2.4 M in THF) was added dropwise over 10 min. After addition was complete, reaction was removed from bath and warmed to RT. After completion, flask was cooled back down to −78° C. and quenched slowly with × mL of water and stirred for 10 min, followed by 3× mL 6 M NaOH, stirring for 10 min, and then 3× mL of water. Filtered through celite and THF removed by rotary evaporation. Extracted with 3×20 mL EtOAc. Combined organic layers were washed with brine and dried over sodium sulfate.

Etherification Procedure: Alcohol was dissolved in DMF and cooled to 0° C. under nitrogen atmosphere. Sodium hydride (60% dispersion) was added and the reaction was stirred for 30 min warming to RT. Cooled back down and alkyl bromide was added and stirred for another 3 h. the reaction was quenched with 20 mL water and extracted with 3×20 mL EtOAc. Combined organic layers were washed with brine and dried over sodium sulfate.

### 1-(Tert-butoxycarbonyl)piperidine-3-carboxylic acid

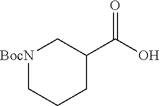 [SMILES: CC(C)(C)OC(=O)N1CCCC(C(=O)O)C1]

Nipecotic acid (1.6343 g, 12.65 mmol) was dissolved in tert-butanol (15 mL) and 1M NaOH (15 mL). Boc₂O (2.79 g, 12.78 mmol) was added and stirred vigorously for 17.3 h. reaction cooled to 0° C. and acidified with 13 mL of 1M HCl. Precipitate collected and washed with cold water to give product as a white solid (2.6784 g, 92%).

### 1-(Tert-butyl) 3-ethyl piperidine-1,3-dicarboxylate

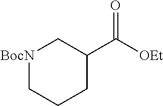 [SMILES: CCOC(=O)C1CCCN(C(=O)OC(C)(C)C)C1]

Nipecotic acid ethyl ester (11.65 mL, 75 mmol), Boc-anhydride (1.5 eq, 24.55 g, 112.5 mmol), Triethylamine (40 mL), Acetonitrile (30 mL), and water (120 mL) were combined in a round bottom flask equipped with a stir bar. To this solution was added DMAP (93.0 mg, 1 mol %) and the reaction was stirred overnight (22 hr). Solvents were removed by rotary evaporator and the oil was purified by flash chromatography (10% EtOAc/Hex) to give 1.9125 g of a clear oil that crystalized overnight in the freezer (74.3 mmol, 99%).

### Tert-butyl 3-(hydroxymethyl)-3-(3-phenylpropyl)piperidine-1-carboxylate

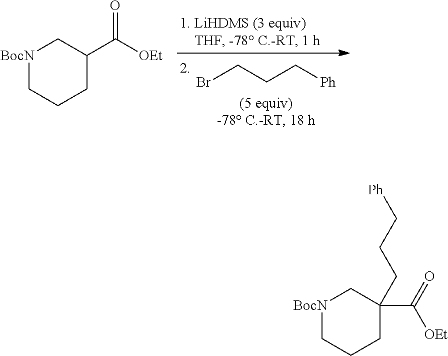 [SMILES: CCOC(=O)C1CCCN(C(=O)OC(C)(C)C)C1 ; BrCCCc1ccccc1 ; CCOC(=O)C1(CCCc2ccccc2)CCCN(C(=O)OC(C)(C)C)C1]

1-(Tert-butyl) 3-ethyl piperidine-1,3-dicarboxylate (1 eq, 1.9125 g, 74.3 mmol) was dissolved in THF (250 mL) in a 3-neck round bottom flask equipped with an addition funnel and placed under nitrogen atmosphere. The solution was cooled to −78° C. and 1M LiHMDS in THF (3 eq, 225 mL) was added via addition funnel over 15 min. After 30 min the reaction was allowed to warm to RT. After an additional 30 min the reaction was cooled back to −78° C. and 3-phenylpropylbromide (5 eq, 375 mmol, 57.2 mL) was added. The reaction was allowed to warm to RT overnight. It was cooled in an ice bath and quenched with aqueous ammonium chloride (˜30 mL). The reaction was concentrated down, diluted with 200 mL EtOAc and washed with 3×100 mL water, 100 mL of brine, and dried over sodium sulfate. Purified by flash chromatography (10% to 20% EtOAc/Hex) to give a yellow oil (25.00 g, 90%).

### Tert-butyl 3-(hydroxymethyl)-3-(3-phenylpropyl)piperidine-1-carboxylate

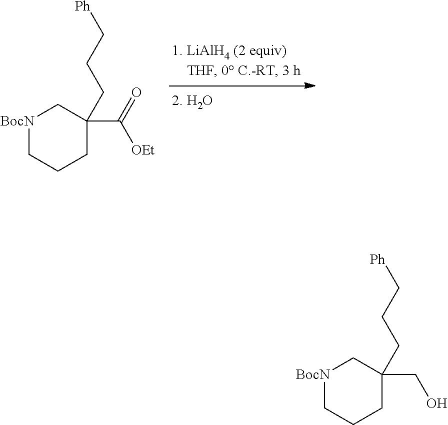 [SMILES: CCOC(=O)C1(CCCc2ccccc2)CCCN(C(=O)OC(C)(C)C)C1 ; CC(C)(C)OC(=O)N1CCCC(CO)(CCCc2ccccc2)C1]

The title compound was prepared by the reduction procedure using 1-(tert-butyl) 3-ethyl 3-(3-phenylpropyl)piperidine-1,3-dicarboxylate (25 g, 66.6 mmol), in 400 mL THF with Lithium aluminum hydride (55.5 mL, 133.2 mmol) added via addition funnel. After 3 hr reaction was quenched with 10 mL of water. Gave a pale-yellow oil that was used directly in the next reaction (quantitative yield). ¹H NMR (600 MHz, CDCl₃): δ 7.31-7.23 (m, 2H), 7.22-7.14 (m, 3H), 3.83 (d, J=13.1 Hz, 1H), 3.48 (dd, J=11.8, 4.5 Hz, 1H), 3.41-3.08 (m, 2H), 2.93 (d, J=121.0 Hz, 1H), 2.68-2.48 (m, 3H), 1.71-1.10 (m, 17H); ¹³C NMR (151 MHz, CDCl₃): δ 156.26, 142.64, 128.46, 128.37, 125.79, 80.01, 62.79, 49.80, 45.79, 38.35, 36.79, 35.60, 30.85, 28.50, 25.08, 21.61.

### (3-(3-Phenylpropyl)piperidin-3-yl)methanol

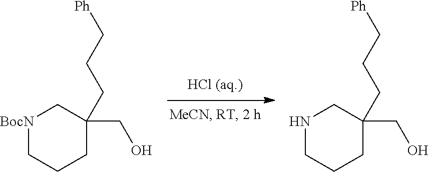 [SMILES: CC(C)(C)OC(=O)N1CCCC(CO)(CCCc2ccccc2)C1 ; OCC1(CCCc2ccccc2)CCCNC1]

Tert-butyl 3-(hydroxymethyl)-3-(3-phenylpropyl)piperidine-1-carboxylate (1.22 g, 3.66 mmol) was dissolved in 70 mL of acetonitrile. 35 mL of 6 M HCl was added and the reaction was stirred for 2 hr. The flask was cooled in an ice bath and 6 M NaOH was added (37 mL). MeCN was removed by rotary evaporator and the product was extracted with 3×20 mL EtOAc. Combined organic layers were washed with brine and dried over sodium sulfate to give a thick, clear oil (747.6 mg, 88%). ¹H NMR (600 MHz, CDCl₃): δ 7.27 (t, J=7.8 Hz, 2H), 7.20-7.15 (m, 3H), 3.65-3.57 (m, 2H), 3.29 (s, 2H), 2.91 (dd, J=11.6, 5.1 Hz, 2H), 2.61 (dd, J=10.8, 3.6 Hz, 1H), 2.56 (ddd, J=8.4, 6.9, 3.8 Hz, 2H), 2.51 (d, J=10.9 Hz, 1H), 1.85-1.77 (m, 1H), 1.65-1.47 (m, 4H), 1.34-1.17 (m, 3H); ¹³C NMR (151 MHz, CDCl₃): δ 142.51, 128.40, 128.35, 125.79, 69.96, 55.11, 46.90, 36.93, 36.82, 35.94, 32.97, 24.99, 22.92.

### (1-(2-Aminopyrimidin-4-yl)-3-phenethylpiperidin-3-yl)methanol

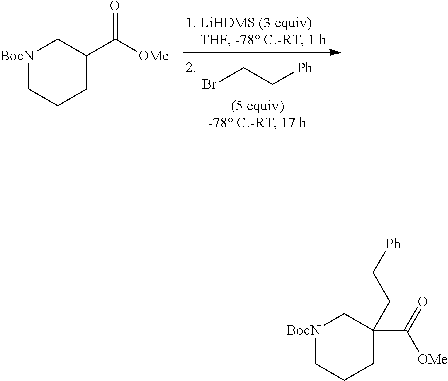 [SMILES: COC(=O)C1CCCN(C(=O)OC(C)(C)C)C1 ; BrCCc1ccccc1 ; COC(=O)C1(CCc2ccccc2)CCCN(C(=O)OC(C)(C)C)C1]

1-(Tert-butyl) 3-methyl piperidine-1,3-dicarboxylate (1.2167 g, 5.00 mmol) was dissolved in dry THF (20 mL) and placed under nitrogen atmosphere. The solution was cooled to −78° C. and 1M LiHMDS in THF (15 mL, 15 mmol) was added slowly over 5 min. Let warm to warm to RT over 1 h. The reaction was cooled back to −78° C. and (2-bromoethyl)benzene (3.414 mL, 25.00 mmol) was added. The reaction was allowed to warm to RT overnight (17 h). It was cooled in an ice bath and quenched with aqueous ammonium chloride (˜5 mL). The reaction was concentrated down, diluted with 100 mL EtOAc and washed with 3×50 mL water, 100 mL of brine, and dried over sodium sulfate. Purified by flash chromatography (Hexane) to give 1-(tert-butyl) 3-methyl 3-phenethylpiperidine-1,3-dicarboxylate as a clear oil (1.7381 g, quantitative yield).

### Methyl 3-(3-phenylpropyl)piperidine-3-carboxylate

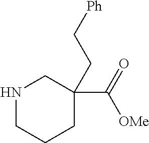 [SMILES: COC(=O)C1(CCc2ccccc2)CCCNC1]

1-(Tert-butyl) 3-methyl 3-(3-phenylpropyl)piperidine-1,3-dicarboxylate (724.4 mg, 2.00 mmol) was dissolved in DCM (20 mL). Trifluoroacetic acid was added and stirred for 1 h. Solution was neutralized with 20 mL aqueous Na₂CO₃ and organic layer was separated. Aqueous layer was washed with 2×20 mL EtOAc. Combined organic layers dried over sodium sulfate and purified by flash chromatography (50% Acetone/Hexane) to give a pale yellow oil (466.9 mg, 89%). ¹H NMR (800 MHz, CDCl₃): δ 7.34 (td, J=7.8, 2.5 Hz, 2H), 7.25 (td, J=6.6, 5.9, 2.8 Hz, 1H), 7.21 (d, J=7.4 Hz, 2H), 3.76 (d, J=3.4 Hz, 3H), 3.44 (d, J=12.1 Hz, 1H), 3.00 (d, J=12.7 Hz, 1H), 2.63 (t, J=6.7 Hz, 3H), 2.48 (dd, J=12.8, 2.6 Hz, 1H), 2.28-2.21 (m, 1H), 1.87 (s, 1H), 1.63-1.52 (m, 5H), 1.48 (dddd, J=15.2, 13.5, 7.9, 3.9 Hz, 1H), 1.39 (td, J=12.6, 4.0 Hz, 1H); ¹³C NMR (201 MHz, CDCl₃): δ 176.42, 141.90, 128.31, 128.29, 125.80, 53.60, 51.64, 46.73, 46.48, 37.91, 36.12, 33.05, 25.67, 24.38.

### Tert-butyl 3-(hydroxymethyl)-3-phenethylpiperidine-1-carboxylate

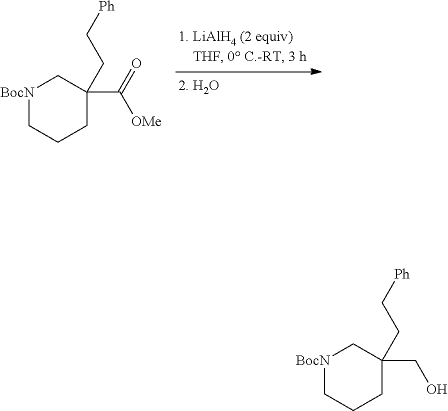 [SMILES: COC(=O)C1(CCc2ccccc2)CCCN(C(=O)OC(C)(C)C)C1 ; CC(C)(C)OC(=O)N1CCCC(CO)(CCc2ccccc2)C1]

Tert-butyl 3-(hydroxymethyl)-3-phenethylpiperidine-1-carboxylate was prepared by the reduction procedure using 1-(tert-butyl) 3-methyl 3-phenethylpiperidine-1,3-dicarboxylate (1.0465 g, 3.01 mmol), in 20 mL THF with Lithium aluminum hydride (2.5 mL, 6.00 mmol). After 3 hr reaction was quenched with 1 mL of water. Purified by flash chromatography on silica (10% Acetone/Hexane) to give a clear oil (233.3 mg, 24%).

### (3-phenethylpiperidin-3-yl)methanol

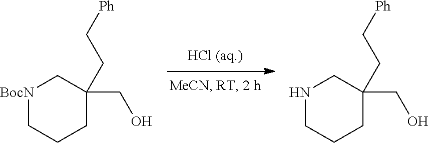 [SMILES: CC(C)(C)OC(=O)N1CCCC(CO)(CCc2ccccc2)C1 ; OCC1(CCc2ccccc2)CCCNC1]

Tert-butyl 3-(hydroxymethyl)-3-phenethylpiperidine-1-carboxylate (233.3 g, 0.73 mmol) was dissolved in 10 mL of acetonitrile. 5 mL of 6 M HCl was added and the reaction was stirred for 2 hr. The flask was cooled in an ice bath and 6 M NaOH was added (5.5 mL) was added. MeCN was removed by rotary evaporator and the product was extracted with 3×10 mL EtOAc. Combined organic layers were washed with brine and dried over sodium sulfate to give (3-phenethylpiperidin-3-yl)methanol as a pale yellow oil (130.5 mg, 82%)

### 3-((benzyloxy)methyl)-3-(3-phenylpropyl)piperidine

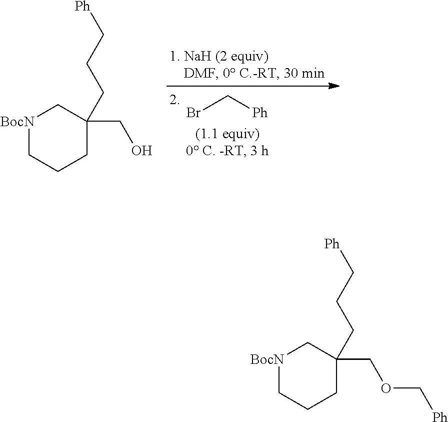 [SMILES: CC(C)(C)OC(=O)N1CCCC(CO)(CCCc2ccccc2)C1 ; BrCc1ccccc1 ; CC(C)(C)OC(=O)N1CCCC(CCCc2ccccc2)(COCc2ccccc2)C1]

Tert-butyl 3-((benzyloxy)methyl)-3-(3-phenylpropyl)piperidine-1-carboxylate was prepared by the etherification procedure using tert-butyl 3-(hydroxymethyl)-3-(3-phenylpropyl)piperidine-1-carboxylate (339.2 mg, 1.02 mmol), sodium hydride (86.1 mg, 2.15 mmol), and benzyl bromide (131 μL, 1.10 mmol) in DMF (6 mL). Purified by flash chromatography on silica (5% Acetone/Hexane) to give a pale yellow oil (267.3 mg, 63%).

### 3-((benzyloxy)methyl)-3-(3-phenylpropyl)piperidine

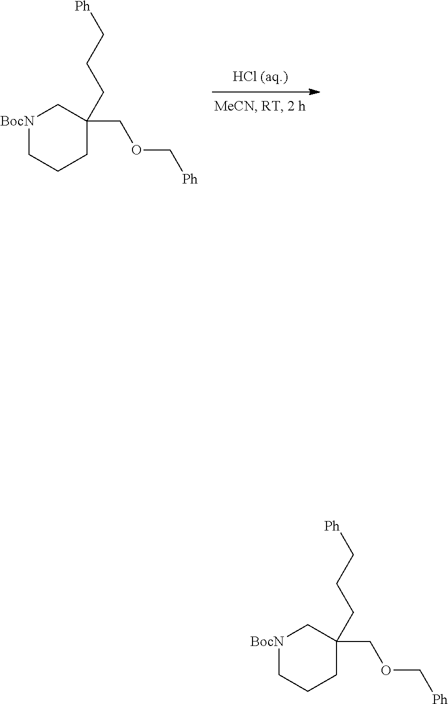 [SMILES: CC(C)(C)OC(=O)N1CCCC(CCCc2ccccc2)(COCc2ccccc2)C1 ; CC(C)(C)OC(=O)N1CCCC(CCCc2ccccc2)(COCc2ccccc2)C1]

Tert-butyl 3-((benzyloxy)methyl)-3-(3-phenylpropyl)piperidine-1-carboxylate (197.7 mg, 0.47 mmol) was dissolved in 10 mL of acetonitrile. 5 mL of 6 M HCl was added and the reaction was stirred for 2 hr. The flask was cooled in an ice bath and 6 M NaOH was added (5.5 mL) was added over 15 min. MeCN was removed by rotary evaporator and the product was extracted with 3×20 mL EtOAc. Combined organic layers were washed with brine and dried over sodium sulfate to give a clear oil (125.1 mg, 83%). ¹H NMR (600 MHz, CDCl₃): δ 7.42-7.34 (m, 4H), 7.32 (ddd, J=7.8, 6.5, 1.7 Hz, 3H), 7.23 (ddt, J=8.9, 2.9, 1.6 Hz, 3H), 4.57-4.49 (m, 2H), 3.42-3.33 (m, 2H), 2.83-2.71 (m, 3H), 2.64 (t, J=7.4 Hz, 2H), 2.60 (d, J=12.4 Hz, 1H), 1.89 (s, 1H), 1.64-1.45 (m, 7H), 1.42-1.33 (m, 1H); ¹³C NMR (151 MHz, CDCl₃): δ 142.70, 138.89, 128.34, 128.25, 128.21, 127.35, 127.34, 125.60, 73.86, 73.18, 53.69, 47.01, 36.77, 36.33, 34.69, 31.33, 25.04, 22.40.

### 3-((Benzyloxy)methyl)piperidine

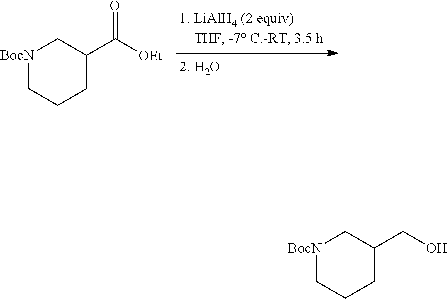 [SMILES: CCOC(=O)C1CCCN(C(=O)OC(C)(C)C)C1 ; CC(C)(C)OC(=O)N1CCCC(CO)C1]

Tert-butyl 3-(hydroxymethyl)piperidine-1-carboxylate was prepared by reduction procedure using 1-(tert-butyl) 3-ethyl piperidine-1,3-dicarboxylate (1.0294 g, 4.00 mmol), in 25 mL THF with lithium aluminum hydride (3.33 mL, 8.00 mmol). After 3.5 h reaction was quenched with 3 mL of water. Gave a white solid (652.6 mg, 76%). ¹H NMR (600 MHz, CDCl₃): δ 3.68 (d, J=102.2 Hz, 2H), 3.42 (d, J=6.8 Hz, 2H), 2.89 (d, J=98.3 Hz, 3H), 1.76-1.69 (m, 1H), 1.60 (d, J=53.3 Hz, 2H), 1.39 (s, 9H), 1.25-1.11 (m, 1H); ¹³C NMR (151 MHz, CDCl₃): δ 155.33, 79.44, 64.18, 46.09, 45.23, 38.06, 28.37, 26.93, 24.01; NMR spectra are consistent with literature reports.¹

### Tert-butyl 3-((benzyloxy)methyl)piperidine-1-carboxylate

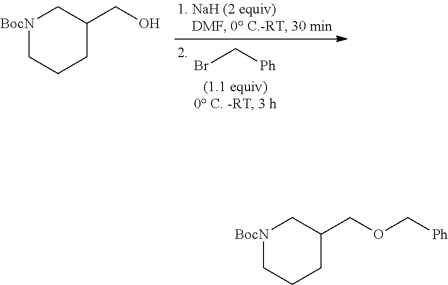 [SMILES: CC(C)(C)OC(=O)N1CCCC(CO)C1 ; BrCc1ccccc1 ; CC(C)(C)OC(=O)N1CCCC(COCc2ccccc2)C1]

Tert-butyl 3-((benzyloxy)methyl)piperidine-1-carboxylate was prepared by the etherification procedure using tert-butyl 3-(hydroxymethyl)piperidine-1-carboxylate (538.0 mg, 2.50 mmol), sodium hydride (223.7 mg, 5.59 mmol), and benzyl bromide (327 μL, 2.75 mmol) in DMF (15 mL). Purified by flash chromatography on silica (10% EtOAc/Hexane) to give a clear oil (636.7 mg, 83%). ¹H NMR (600 MHz, CDCl₃): δ 7.32 (dd, J=5.4, 2.1 Hz, 4H), 7.26 (tt, J=6.0, 2.6 Hz, 1H), 4.48 (s, 2H), 4.02 (s, 1H), 3.88 (dt, J=13.3, 4.2 Hz, 1H), 3.37-3.26 (m, 2H), 2.80 (t, J=10.7 Hz, 1H), 2.65 (s, 1H), 1.86-1.75 (m, 2H), 1.62 (dt, J=13.4, 3.8 Hz, 1H), 1.45 (s, 10H), 1.22 (q, J=10.0, 9.6 Hz, 1H); ¹³C NMR (151 MHz, CDCl₃): δ 154.91, 138.49, 128.32, 127.48, 127.43, 79.19, 73.01, 72.68, 47.29, 44.33, 36.36, 27.59, 24.52.

### 3-((benzyloxy)methyl)piperidine

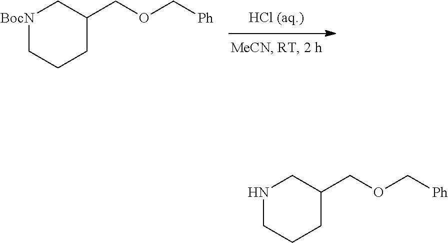 [SMILES: CC(C)(C)OC(=O)N1CCCC(COCc2ccccc2)C1 ; c1ccc(COCC2CCCNC2)cc1]

Tert-butyl 3-((benzyloxy)methyl)piperidine-1-carboxylate (617.1 mg, 2.02 mmol) was dissolved in 40 mL of acetonitrile. 20 mL of 6 M HCl was added and the reaction was stirred for 2 hr. The flask was cooled in an ice bath and 6 M NaOH was added (22 mL) was added slowly. MeCN was removed by rotary evaporator and the product was extracted with 3×30 mL EtOAc. Combined organic layers were dried over sodium sulfate to give a pale yellow oil (348.8 mg, 84%). ¹H NMR (600 MHz, CDCl₃): 1H NMR (600 MHz, Chloroform-d) δ 7.28-7.20 (m, 4H), 7.17 (ddd, J=8.7, 5.7, 2.6 Hz, 1H), 4.38 (d, J=4.1 Hz, 2H), 3.27-3.16 (m, 2H), 3.07 (d, J=12.2 Hz, 1H), 2.90 (dt, J=12.1, 3.8 Hz, 1H), 2.44 (td, J=11.8, 3.0 Hz, 1H), 2.26 (dd, J=12.2, 10.1 Hz, 1H), 2.05 (s, 1H), 1.78-1.68 (m, 2H), 1.55 (dq, J=13.7, 3.5 Hz, 1H), 1.37 (dtt, J=13.2, 11.8, 3.9 Hz, 1H), 1.04 (dtd, J=12.8, 11.6, 11.1, 4.7 Hz, 1H).

### (S)-3-((Benzyloxy)methyl)piperidine

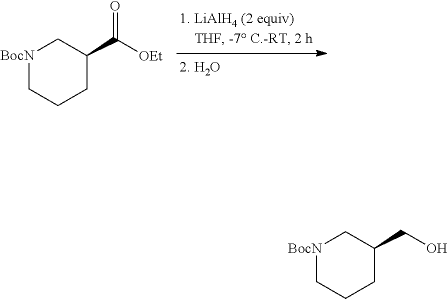 [SMILES: CCOC(=O)[C@H]1CCCN(C(=O)OC(C)(C)C)C1 ; CC(C)(C)OC(=O)N1CCC[C@H](CO)C1]

Tert-butyl (S)-3-(hydroxymethyl)piperidine-1-carboxylate was prepared by the reduction procedure using 1-Boc-L-nipecotic acid (1.1496 g, 5.01 mmol), in 50 mL THF with Lithium aluminum hydride (4.17 mL, 10.00 mmol). After 2 hr reaction was quenched with 3 mL of water. Gave a white solid (652.6 mg, 76%). ¹H NMR (600 MHz, CDCl₃): δ 3.89-3.54 (m, 2H), 3.40 (d, J=6.8 Hz, 2H), 3.10 (s, 1H), 2.81 (d, J=86.9 Hz, 2H), 1.72 (dq, J=13.5, 4.5 Hz, 1H), 1.63 (ddt, J=10.7, 7.5, 3.9 Hz, 1H), 1.56 (dt, J=13.9, 4.6 Hz, 1H), 1.38 (s, 9H), 1.23-1.10 (m, 1H); ¹³C NMR (151 MHz, CDCl₃): δ 155.16, 79.43, 64.35, 46.31, 44.85, 38.13, 28.36, 26.94, 24.04; NMR spectra are consistent with literature reports.¹

### Tert-butyl (S)-3-((benzyloxy)methyl)piperidine-1-carboxylate

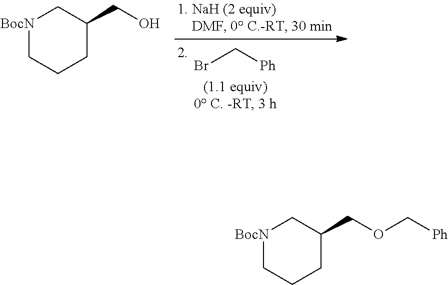 [SMILES: CC(C)(C)OC(=O)N1CCC[C@H](CO)C1 ; BrCc1ccccc1 ; CC(C)(C)OC(=O)N1CCC[C@H](COCc2ccccc2)C1]

Tert-butyl (S)-3-((benzyloxy)methyl)piperidine-1-carboxylate was prepared by the etherification procedure using tert-butyl (S)-3-(hydroxymethyl)piperidine-1-carboxylate (392.8 mg, 1.82 mmol), sodium hydride (173.2 mg, 4.33 mmol), and benzyl bromide (239 μL, 2.01 mmol) in DMF (10 mL). Purified by flash chromatography on silica (10% EtOAc/Hexane) to give a clear oil (495.1 mg, 89%). ¹H NMR (600 MHz, CDCl₃): δ 7.27 (d, J=5.1 Hz, 4H), 7.20 (hept, J=3.7 Hz, 1H), 4.43 (s, 2H), 4.00 (d, J=37.5 Hz, 1H), 3.84 (d, J=12.6 Hz, 1H), 3.31-3.22 (m, 2H), 2.76 (t, J=11.0 Hz, 1H), 2.63 (s, 1H), 1.82-1.69 (m, 2H), 1.56 (dt, J=13.3, 3.9 Hz, 1H), 1.42 (s, 10H), 1.17 (dtd, J=14.3, 10.9, 3.9 Hz, 1H); ¹³C NMR (151 MHz, CDCl₃): δ 154.60, 138.30, 128.07, 127.23, 127.16, 78.86, 72.75, 72.42, 47.04, 44.10, 36.15, 28.24, 27.34, 24.30.

### (S)-3-((benzyloxy)methyl)piperidine

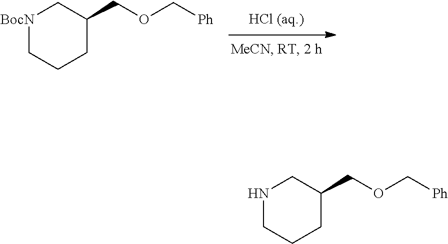 [SMILES: CC(C)(C)OC(=O)N1CCC[C@H](COCc2ccccc2)C1 ; c1ccc(COC[C@H]2CCCNC2)cc1]

Tert-butyl (S)-3-((benzyloxy)methyl)piperidine-1-carboxylate (482.7 mg, 1.58 mmol) was dissolved in 30 mL of acetonitrile. 15 mL of 6 M HCl was added and the reaction was stirred for 2 hr. The flask was cooled in an ice bath and 6 M NaOH was added (17 mL) was added slowly. MeCN was removed by rotary evaporator and the product was extracted with 3×30 mL EtOAc. Combined organic layers were washed with brine and dried over sodium sulfate to give a pale yellow oil (311.5 mg, 96%). ¹H NMR (600 MHz, CDCl₃): δ 7.28-7.20 (m, 4H), 7.17 (qq, J=5.0, 2.7 Hz, 1H), 4.38 (d, J=3.9 Hz, 2H), 3.25-3.16 (m, 2H), 3.07 (d, J=11.9 Hz, 1H), 2.90 (dt, J=12.0, 3.4 Hz, 1H), 2.65 (s, 1H), 2.44 (td, J=11.8, 3.0 Hz, 1H), 2.27 (dd, J=12.1, 10.1 Hz, 1H), 1.79-1.67 (m, 2H), 1.55 (dp, J=13.7, 3.4 Hz, 1H), 1.39 (dtt, J=13.3, 11.8, 3.9 Hz, 1H), 1.04 (tdd, J=12.7, 11.0, 4.0 Hz, 1H); ¹³C NMR (151 MHz, CDCl₃): δ 138.32, 128.00, 127.13, 127.13, 73.35, 72.64, 49.77, 46.52, 37.06, 27.73, 25.61.

### (R)-3-((Benzyloxy)methyl)piperidine

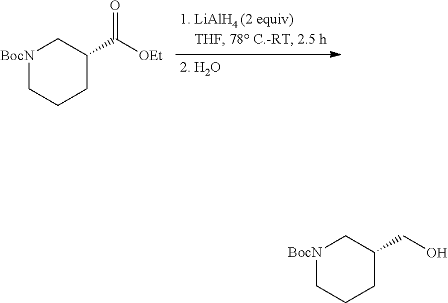 [SMILES: CCOC(=O)[C@@H]1CCCN(C(=O)OC(C)(C)C)C1 ; CC(C)(C)OC(=O)N1CCC[C@@H](CO)C1]

Tert-butyl (R)-3-(hydroxymethyl)piperidine-1-carboxylate was prepared by the reduction procedure using 1-Boc-D-nipecotic acid (2.2947 g, 10.00 mmol), in 60 mL THF with lithium aluminum hydride (8.334 mL, 20.00 mmol). After 2.5 hr reaction was quenched with 3 mL of water. Gave a white solid (1.69 g, 78%). ¹H NMR (600 MHz, CDCl₃): δ 3.81-3.46 (m, 3H), 3.26 (dd, J=6.6, 3.6 Hz, 2H), 2.83-2.44 (m, 2H), 1.60 (dq, J=13.3, 4.4 Hz, 1H), 1.55-1.39 (m, 2H), 1.25 (s, 9H), 1.03 (td, J=10.8, 10.3, 6.9 Hz, 1H); ¹³C NMR (151 MHz, CDCl₃): δ 3.83-3.46 (m, 3H), 3.27 (dd, J=6.7, 3.6 Hz, 2H), 2.86-2.42 (m, 2H), 1.61 (dt, J=13.3, 4.4 Hz, 1H), 1.55-1.40 (m, 2H), 1.35-1.14 (m, 9H), 1.11-0.97 (m, 1H); NMR spectra are consistent with literature reports.¹

### Tert-butyl (R)-3-((benzyloxy)methyl)piperidine-1-carboxylate

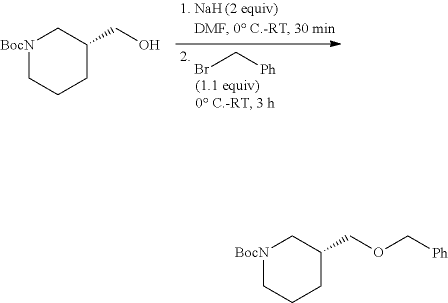 [SMILES: CC(C)(C)OC(=O)N1CCC[C@@H](CO)C1 ; BrCc1ccccc1 ; CC(C)(C)OC(=O)N1CCC[C@@H](COCc2ccccc2)C1]

Tert-butyl (R)-3-((benzyloxy)methyl)piperidine-1-carboxylate was prepared by the etherification procedure using tert-butyl (R)-3-(hydroxymethyl)piperidine-1-carboxylate (1.69 g, 7.85 mmol), sodium hydride (698.4 mg, 17.47 mmol), and benzyl bromide (1.03 mL, 8.63 mmol) in DMF (40 mL). Purified by flash chromatography on silica (10% EtOAc/Hexane) to give a clear oil (967.1 mg, 40%). ¹H NMR (600 MHz, CDCl₃): δ 7.26 (d, J=4.8 Hz, 4H), 7.20 (q, J=4.4 Hz, 1H), 4.42 (s, 2H), 4.00 (s, 1H), 3.83 (d, J=13.2 Hz, 1H), 3.26 (q, J=9.2, 7.3 Hz, 2H), 2.75 (t, J=12.8 Hz, 1H), 2.62 (s, 1H), 1.81-1.69 (m, 2H), 1.56 (dt, J=13.2, 3.9 Hz, 1H), 1.49-1.28 (m, 10H), 1.17 (dtd, J=14.3, 11.1, 3.9 Hz, 1H); ¹³C NMR (151 MHz, CDCl₃): δ 154.66, 138.34, 128.11, 127.27, 127.21, 78.91, 72.80, 72.47, 46.99, 43.98, 36.20, 28.29, 24.37.

### (R)-3-((benzyloxy)methyl)piperidine

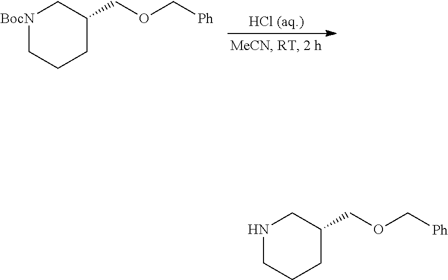 [SMILES: CC(C)(C)OC(=O)N1CCC[C@@H](COCc2ccccc2)C1 ; "HCl (aq.)" ; c1ccc(COC[C@@H]2CCCNC2)cc1]

Tert-butyl (R)-3-((benzyloxy)methyl)piperidine-1-carboxylate (922.7 mg, 3.02 mmol) was dissolved in 60 mL of acetonitrile. 30 mL of 6 M HCl was added and the reaction was stirred for 2 hr. The flask was cooled in an ice bath and 6 M NaOH was added (34 mL) was added slowly. MeCN was removed by rotary evaporator and the product was extracted with 3×50 mL EtOAc. Combined organic layers were washed with brine and dried over sodium sulfate to give a pale yellow oil (662.7 mg, quantitative yield). ¹H NMR (600 MHz, CDCl₃): δ 7.23 (d, J=5.6 Hz, 4H), 7.16 (dq, J=6.0, 3.7 Hz, 1H), 4.38 (d, J=3.8 Hz, 2H), 3.25-3.17 (m, 2H), 3.15 (s, 1H), 3.08 (dd, J=12.3, 3.8 Hz, 1H), 2.91 (d, J=12.4 Hz, 1H), 2.44 (td, J=11.9, 3.1 Hz, 1H), 2.28 (t, J=11.5, 11.1 Hz, 1H), 1.77 (ddt, J=10.5, 6.4, 3.5 Hz, 1H), 1.71 (d, J=13.1 Hz, 1H), 1.55 (dp, J=14.1, 3.6 Hz, 1H), 1.41 (qt, J=12.0, 4.0 Hz, 1H), 1.05 (qd, J=12.2, 4.0 Hz, 1H); ¹³C NMR (151 MHz, CDCl₃): δ 138.05, 127.70, 126.83, 126.81, 72.98, 72.32, 49.32, 46.10, 36.62, 27.34, 25.15.

### N-Phenethylpiperidine-3-carboxamide

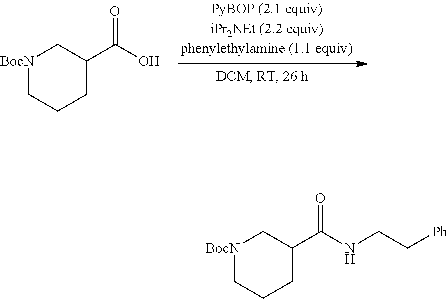 [SMILES: CC(C)(C)OC(=O)N1CCCC(C(=O)O)C1 ; CC(C)(C)OC(=O)N1CCCC(C(=O)NCCc2ccccc2)C1]

1-Tert-butoxycarbonyl)piperidine-3-carboxylic acid (344.5 mg, 1.50 mmol) was dissolved in DCM (15 mL) and DIPEA (575 μL, 3.30 mmol). PyBOP (1.64 g, 3.15 mmol) was added under nitrogen atmosphere followed by phenethylamine (208 μL, 1.65 mmol). After 26 h, reaction was washed with 3×10 mL water. Combined aqueous layers were back extracted with 10 mL DCM. Combined organic layers were washed with brine and dried over sodium sulfate. Purified by flash column chromatography on silica (10% to 0% Hexane/DCM) followed by alumina (EtOAc) to give tert-butyl 3-(phenethylcarbamoyl)piperidine-1-carboxylate as a clear oil (186.6 mg, 38%).

### N-phenethylpiperidine-3-carboxamide

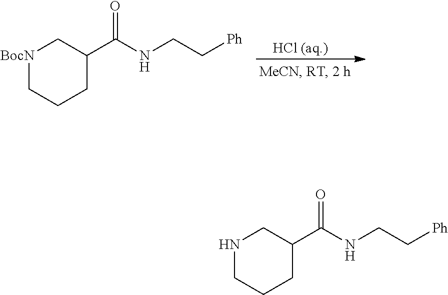 [SMILES: CC(C)(C)OC(=O)N1CCCC(C(=O)NCCc2ccccc2)C1 ; "HCl (aq.)" ; O=C(NCCc1ccccc1)C1CCCNC1]

Tert-butyl 3-(phenethylcarbamoyl)piperidine-1-carboxylate (186.6 mg, 0.56 mmol) was dissolved in 10 mL of acetonitrile. 5 mL of 6 M HCl was added and the reaction was stirred for 1.5 hr. The flask was cooled in an ice bath and 6 M NaOH was added (5.5 mL) was added over 15 min. MeCN was removed by rotary evaporator and the product was extracted with 3×20 mL EtOAc. Combined organic layers were washed with brine and dried over sodium sulfate to give N-phenethylpiperidine-3-carboxamide as a pale yellow oil (129.6 mg, 99%).

### 1-(2-Aminopyrimidin-4-yl)-N-benzylpiperidine-3-carboxamide

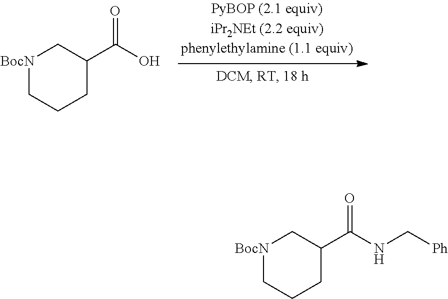 [SMILES: CC(C)(C)OC(=O)N1CCCC(C(=O)O)C1 ; CC(C)(C)OC(=O)N1CCCC(C(=O)NCc2ccccc2)C1]

1-(Tert-butoxycarbonyl)piperidine-3-carboxylic acid (345.2 mg, 1.51 mmol) dissolved in DCM (15 mL) and DIPEA (575 μL, 3.30 mmol). PyBOP (1.65 g, 3.17 mmol) added under nitrogen atmosphere followed by benzylamine (180 μL, 1.65 mmol). After 18 h, reaction was washed with 3×10 mL water. Combined aqueous layers were back extracted with 10 mL DCM. Combined organic layers were washed with brine and dried over sodium sulfate. Purified by flash column chromatography on silica (10% to 33% Acetone/Hexane) to give tert-butyl 3-(benzylcarbamoyl)piperidine-1-carboxylate as a clear oil (274.9 mg, 56%). ¹H NMR (600 MHz, CDCl₃): δ 7.29-7.22 (m, 2H), 7.19 (tt, J=10.1, 2.3 Hz, 3H), 6.79 (s, 1H), 4.34 (s, 2H), 4.05-3.52 (m, 2H), 3.18-2.59 (m, 2H), 2.26 (tt, J=9.5, 4.3 Hz, 1H), 1.97-1.63 (m, 2H), 1.63-1.50 (m, 1H), 1.50-1.17 (m, 10H)

### N-benzylpiperidine-3-carboxamide

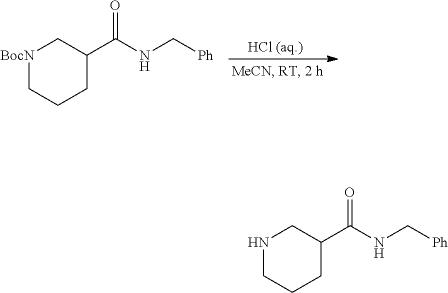 [SMILES: CC(C)(C)OC(=O)N1CCCC(C(=O)NCc2ccccc2)C1 ; O=C(NCc1ccccc1)C1CCCNC1]

Tert-butyl 3-(benzylcarbamoyl)piperidine-1-carboxylate (274.9 mg, 0.86 mmol) was dissolved in 16 mL of acetonitrile. 8 mL of 6 M HCl was added and the reaction was stirred for 2 hr. The flask was cooled in an ice bath and 6 M NaOH was added (9 mL) was added over 15 min. MeCN was removed by rotary evaporator and the product was extracted with 3×20 mL EtOAc. Combined organic layers were washed with brine and dried over sodium sulfate to give N-benzylpiperidine-3-carboxamide as a clear oil (188.9 mg, quant.).

### (4-(3-phenylpropyl)piperidin-4-yl)methanol hydrochloride

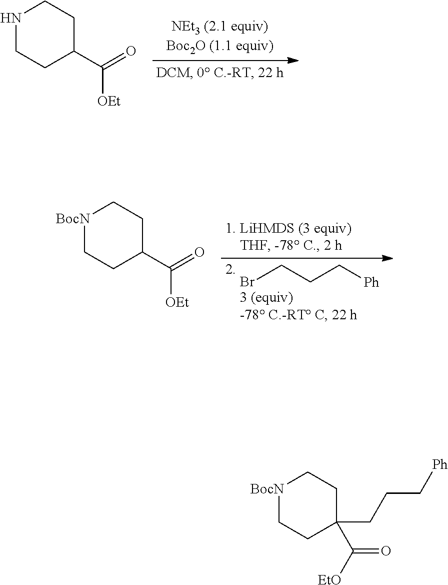 [SMILES: CCOC(=O)C1CCNCC1 ; CCOC(=O)C1CCN(C(=O)OC(C)(C)C)CC1 ; BrCCCc1ccccc1 ; CCOC(=O)C1(CCCc2ccccc2)CCN(C(=O)OC(C)(C)C)CC1]

Ethyl piperidine-4-carboxylate (6.2 mL, 40 mmol) was added to a purged flask of DCM (250 mL) in an ice bath. TEA (11.7 mL, 84.0 mmol) was then added, followed by a solution of Boc₂O (9.6 g, 44 mmol) in DCM (30 mL). The mixture was left to stir for 20 min at 0° C. The mixture was then removed from the ice bath and stirred for 22 h, warming to RT. The reaction mixture was then concentrated, diluted with EtOAc (60 mL) and washed with H₂O (3×20 mL). The water layer was back extracted with EtOAc (20 mL). The organic layers were combined, dried, filtered, concentrated. The residue was then diluted with THF (20 mL) and the resultant solution was slowly added via addition funnel to a solution of LiHMDS (120 mL, 1 M soln, 120 mmol) in THF (100 mL) that was stirring at −78° C. THF (10 mL) was used to rinse the funnel. The mixture was allowed to stir at −78° C. for 2 h before 1-bromo-3-phenylpropane (18.2 mL, 120 mmol) was slowly added. The mixture was left to stir for 20 min at −78° C. before the bath was removed and the mixture was allowed to stir, warming to RT, for another 22 h. The reaction was quenched with the addition of NH₄Cl (25 mL) and allowed to stir for 1 h. The reaction mixture was then filtered and concentrated before being diluted with H₂O (40 mL) and extracted with EtOAc (80 mL). Addition EtOAc (2×80 mL) was used to extract the aqueous layer. The organic layers were collected and combined before being dried, filtered and concentrated. The residue was purified by silica column chromatography (2% NEt₃:hexane followed by 1% NEt₃:hexane) to give the product of the sequence as an orange oil (12 g, 80% yield over two steps). ¹H NMR (600 MHz, CDCl₃): δ 7.28-7.25 (m, 2H), 7.19-7.16 (m, 1H), 7.14-7.12 (m, 2H), 4.15 (q, J=7.1 Hz, 2H), 3.84 (d, J=13.7 Hz, 2H), 2.85 (t, J=12.5 Hz, 2H), 2.56 (p, J=3.1 Hz, 2H), 2.07 (dt, J=13.0, 2.7 Hz, 2H), 1.53 (d, J=3.8 Hz, 4H), 1.44 (s, 9H), 1.31 (ddd, J=13.5, 11.6, 4.6 Hz, 2H), 1.23 (t, J=7.1 Hz, 3H); ¹³C NMR (151 MHz, CDCl₃): δ 175.6, 155.0, 142.0, 128.46, 128.45, 126.0, 79.5, 60.6, 45.6, 41.5, 39.9, 36.1, 33.5, 28.6, 25.7, 14.4; HRMS (ESI/LC-Q-TOF): m/z calcd for C₂₂H₃₃NO₄ [M+Na]⁺: 398.2302; found: 398.2307.

### (4-(3-phenylpropyl)piperidin-4-yl)methanol

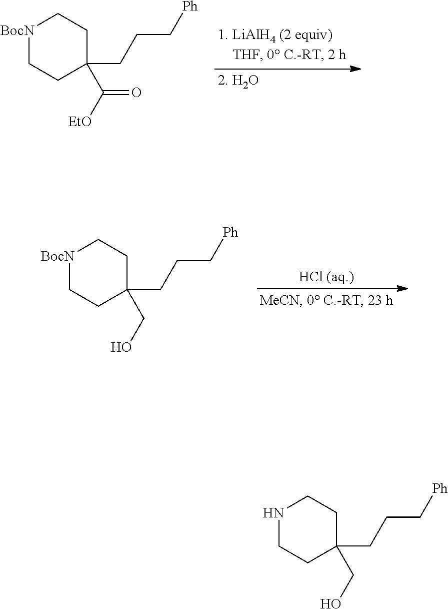 [SMILES: CCOC(=O)C1(CCCc2ccccc2)CCN(C(=O)OC(C)(C)C)CC1 ; CC(C)(C)OC(=O)N1CCC(CO)(CCCc2ccccc2)CC1 ; OCC1(CCCc2ccccc2)CCNCC1]

1-(tert-butyl) 4-ethyl 4-(3-phenylpropyl)piperidine-1,4-dicarboxylate (11 g, 29 mmol) was dissolved in THF (100 mL) and cooled to 0° C. LiAlH₄ (24 mL, 2.4 M soln in THF, 58 mmol) was slowly added and the mixture was allowed to stir for 20 min at 0° C. before being allowed to warm to RT with stirring for 2 h. Following this reaction time, the mixture was cooled to 0° C. before quenching with the sequential addition of H₂O (3 mL), NaOH (1 M, 3 mL), and H₂O (9 mL). The mixture was allowed to stir more and was then filtered through a plug of celite with EtOAc. The filtrate was concentrated in vacuo and the residue was moved to the next step without further purification. The residue was dissolved in MeCN (300 mL) and cooled to 0° C. Aqueous HCl (244 mL, 6 M soln) was slowly added, and the resulting mixtures was allowed to stir for 20 min at 0° C. before being allowed to warm to RT. After 23 h, the solution was brought back down to 0° C. and NaOH (250 mL, 6 M soln) was added to reach a pH of -10. The mixture was concentrated in vacuo and then extracted with DCM (3×200 mL). The organic layers were combined, dried, filtered and concentrated.

### (4-(3-phenylpropyl)piperidin-4-yl)methanol hydrochloride

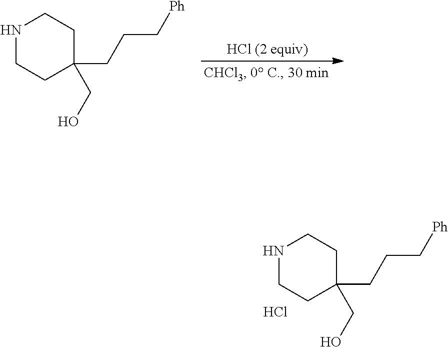 [SMILES: OCC1(CCCc2ccccc2)CCNCC1 ; Cl ; OCC1(CCCc2ccccc2)CCNCC1]

The residue was dissolved in CHCl₃ (70 mL) and brought to 0° C. Ethereal HCl (29 mL, 2 M soln) was slowly added and the mixture was allowed to stir for 30 min. The mixture was then concentrated. The material was then washed with cold EtOAc to give the product as a pale yellow solid (6.3 g, 80% yield over three steps). ¹H NMR (600 MHz, CD₃OD): δ 7.25 (t, J=7.5 Hz, 2H), 7.20 (d, J=7.1 Hz, 2H), 7.15 (t, J=7.3 Hz, 1H), 3.44 (s, 2H), 3.16 (ddd, J=12.1, 7.6, 4.1 Hz, 2H), 3.07 (ddd, J=12.8, 8.6, 4.0 Hz, 2H), 2.62 (t, J=7.5 Hz, 2H), 1.72 (ddd, J=14.8, 8.6, 4.1 Hz, 2H), 1.62-1.55 (m, 4H), 1.49-1.44 (m, 2H); ¹³C NMR (201 MHz, CD₃OD): δ 143.6, 129.5, 129.3, 126.8, 66.7, 41.3, 37.4, 36.0, 34.8, 29.7, 26.0; HRMS (ESI/LC-Q-TOF): m/z calcd for C₁₅H₂₃NO [M+H]⁺: 234.1852; found: 234.1860.

### Example 6: General Procedure 1: Coupling of Amines to Aryl Chlorides

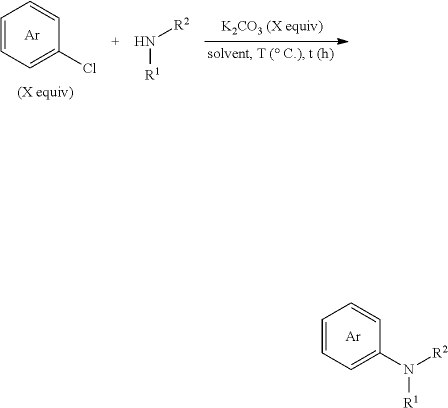

An aryl chloride, an amine, K₂CO₃, and solvent were added to a pressure vial. The vial was sealed with a screw cap, having sometimes been flushed with N₂ and placed in a preheated oil bath with stirring. After the indicated reaction time, the vial was removed from the bath and the mixture was allowed to cool. The reaction mixture was diluted with EtOAc and the resulting solution was washed with H₂O (sometimes basified) multiple times. The organic layers were sometimes back extracted and washed with brine. The organic layer was collected, dried with a drying agent, filtered, and concentrated. Purification of the residue by the indicated method(s) yielded the corresponding product.

### (1-(2-Aminopyrimidin-4-yl)-3-(3-phenylpropyl)piperidin-3-yl)methanol (PLH1215)

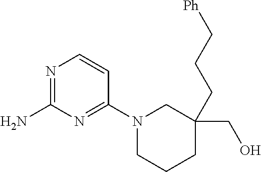 [SMILES: Nc1nccc(N2CCCC(CO)(CCCc3ccccc3)C2)n1]

The title compound was prepared according to General Procedure 1 from (3-(3-phenylpropyl)piperidin-3-yl)methanol (747.6 mg, 3.20 mmol) and 2-amino-4-chloropyridine (456.0 mg, 3.52 mmol) using K₂CO₃ (663.5 mg, 4.80 mmol) in DMSO (6.0 mL) at 120° C. for 18.0 h and was purified by flash column chromatography on silica (10% to 0% Hexane/Acetone) to give a pale yellow solid (693.4 mg, 66%). ¹H NMR (600 MHz, CDCl₃): δ 7.81 (d, J=6.2 Hz, 1H), 7.29-7.25 (m, 2H), 7.17 (ddt, J=7.1, 3.4, 1.5 Hz, 3H), 5.92 (d, J=6.3 Hz, 1H), 4.85 (s, 2H), 4.32 (d, J=13.6 Hz, 1H), 4.06 (s, 1H), 3.81 (d, J=13.1 Hz, 1H), 3.40 (d, J=11.1 Hz, 1H), 3.22 (d, J=11.8 Hz, 1H), 3.03 (t, J=11.4 Hz, 1H), 2.66-2.53 (m, 3H), 1.67 (dddd, J=13.9, 10.4, 6.9, 1.6 Hz, 1H), 1.63-1.47 (m, 5H), 1.41 (ddd, J=14.9, 12.6, 6.0 Hz, 1H), 1.22 (dd, J=12.5, 9.2 Hz, 1H).

### (1-(2-Aminopyrimidin-4-yl)-3-phenethylpiperidin-3-yl)methanol (PLH1304)

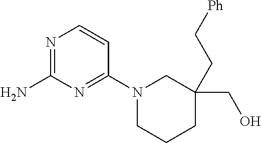 [SMILES: Nc1nccc(N2CCCC(CO)(CCc3ccccc3)C2)n1]

The title compound was prepared according to General Procedure 1 from (3-phenethylpiperidin-3-yl)methanol (130.5 mg, 0.60 mmol) and 2-amino-4-chloropyridine (85.1 mg, 0.66 mmol) using K₂CO₃ (124.6 mg, 0.90 mmol) in DMSO (1.2 mL) at 120° C. for 18.6 h and was purified by flash column chromatography on silica (10% to 2% Hexane/Acetone) to give a white foamy solid (119.9 mg, 65%). ¹H NMR (600 MHz, CDCl₃): δ 7.83 (d, J=6.2 Hz, 1H), 7.30-7.26 (m, 2H), 7.22-7.16 (m, 3H), 5.97 (d, J=6.3 Hz, 1H), 4.79 (s, 2H), 4.39 (d, J=13.7 Hz, 1H), 3.87 (d, J=13.8 Hz, 1H), 3.49 (d, J=12.0 Hz, 1H), 3.34 (d, J=12.0 Hz, 1H), 3.09 (tt, J=10.3, 4.6 Hz, 1H), 2.69 (td, J=13.6, 12.8, 6.0 Hz, 2H), 2.61 (td, J=12.9, 5.0 Hz, 1H), 1.87 (ddd, J=13.7, 12.6, 5.1 Hz, 1H), 1.70-1.51 (m, 4H), 1.50-1.42 (m, 1H); ¹³C NMR (151 MHz, CDCl₃): δ 162.49, 162.02, 156.77, 142.87, 128.47, 128.44, 125.82, 93.98, 62.49, 49.86, 46.18, 39.06, 38.32, 31.30, 29.63, 21.77; HRMS (ESI/LC-Q-TOF): m/z cald for C₁₈H₂₄N₄O [M+H]⁺: 313.2030; found 313.2026.

### 1-(2-Aminopyrimidin-4-yl)-N-benzylpiperidine-3-carboxamide (PLH1290)

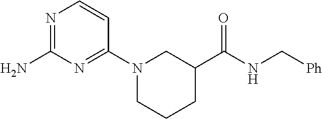 [SMILES: Nc1nccc(N2CCCC(C(=O)NCc3ccccc3)C2)n1]

The title compound was prepared according to General Procedure 1 from N-benzylpiperidine-3-carboxamide (188.6 mg, 0.86 mmol) and 2-amino-4-chloropyridine (158.0 mg, 0.96 mmol) using K₂CO₃ (180.2 mg, 1.30 mmol) in DMSO (2.0 mL) at 120° C. for 15.8 h and was purified by flash column chromatography on silica (1% to 10% MeOH/DCM) to give a white solid (247.5 mg, 92%). ¹H NMR (600 MHz, CDCl₃): δ 7.63 (t, J=5.9 Hz, 1H), 7.58 (d, J=6.3 Hz, 1H), 7.16 (t, J=7.2 Hz, 2H), 7.10 (dd, J=13.9, 7.0 Hz, 3H), 5.78 (d, J=6.5 Hz, 1H), 5.33 (s, 2H), 4.29 (dd, J=14.9, 6.0 Hz, 1H), 4.19 (dd, J=14.9, 5.5 Hz, 1H), 4.11 (d, J=11.7 Hz, 1H), 3.84 (s, 1H), 3.19 (dd, J=13.6, 9.6 Hz, 1H), 2.81 (t, J=10.9 Hz, 1H), 2.29 (tt, J=9.1, 4.0 Hz, 1H), 1.78 (qd, J=10.3, 9.4, 4.1 Hz, 2H), 1.53 (dq, J=12.7, 3.9 Hz, 1H), 1.31 (qd, J=9.7, 5.1 Hz, 1H); ¹³C NMR (151 MHz, CDCl₃): δ 173.09, 161.90, 161.78, 155.04, 138.30, 128.30, 127.30, 126.97, 93.86, 45.74, 44.34, 42.96, 42.18, 27.77, 23.81; HRMS (ESI/LC-Q-TOF): m/z cald for C₁₇H₂₁N₅O [M+H]⁺: 312.1826; found 312.1825.

### 1-(2-Aminopyrimidin-4-yl)-N-phenethylpiperidine-3-carboxamide (PLH1285)

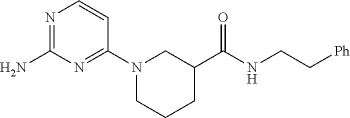 [SMILES: Nc1nccc(N2CCCC(C(=O)NCCc3ccccc3)C2)n1]

The title compound was prepared according to General Procedure 1 from N-phenethylpiperidine-3-carboxamide (129.6 mg, 0.56 mmol) and 2-amino-4-chloropyridine (80.3 mg, 0.62 mmol) using K₂CO₃ (116.6 mg, 0.84 mmol) in DMSO (1.0 mL) at 120° C. for 18.7 h and was purified by flash column chromatography on silica (5% to 10% MeOH/DCM) to give a white solid (106.1 mg, 58%). ¹H NMR (600 MHz, CDCl₃): δ 7.69 (d, J=6.2 Hz, 1H), 7.23 (t, J=7.5 Hz, 2H), 7.16 (t, J=7.5 Hz, 1H), 7.11 (d, J=7.6 Hz, 2H), 6.85 (t, J=5.9 Hz, 1H), 5.84 (d, J=6.3 Hz, 1H), 5.15 (s, 3H), 4.05 (d, J=12.6 Hz, 1H), 3.83 (d, J=13.2 Hz, 1H), 3.43 (dhept, J=20.3, 6.7 Hz, 2H), 3.33 (dd, J=13.7, 9.1 Hz, 1H), 2.96 (ddd, J=13.6, 10.3, 3.3 Hz, 1H), 2.74 (t, J=7.2 Hz, 2H), 2.25 (tt, J=9.1, 4.1 Hz, 1H), 1.86 (ddt, J=13.8, 9.9, 5.0 Hz, 1H), 1.80 (dq, J=13.5, 4.6 Hz, 1H), 1.56 (dq, J=13.5, 4.4 Hz, 1H), 1.43-1.30 (m, 1H); ¹³C NMR (151 MHz, CDCl₃): δ 7.69 (d, J=6.2 Hz, 1H), 7.23 (t, J=7.5 Hz, 2H), 7.16 (t, J=7.5 Hz, 1H), 7.11 (d, J=7.6 Hz, 2H), 6.85 (t, J=5.9 Hz, 1H), 5.84 (d, J=6.3 Hz, 1H), 5.15 (s, 3H), 4.05 (d, J=12.6 Hz, 1H), 3.83 (d, J=13.2 Hz, 1H), 3.43 (dhept, J=20.3, 6.7 Hz, 2H), 3.33 (dd, J=13.7, 9.1 Hz, 1H), 2.96 (ddd, J=13.6, 10.3, 3.3 Hz, 1H), 2.74 (t, J=7.2 Hz, 2H), 2.25 (tt, J=9.1, 4.1 Hz, 1H), 1.86 (ddt, J=13.8, 9.9, 5.0 Hz, 1H), 1.80 (dq, J=13.5, 4.6 Hz, 1H), 1.56 (dq, J=13.5, 4.4 Hz, 1H), 1.43-1.30 (m, 1H); HRMS (ESI/LC-Q-TOF): m/z cald for C₁₈H₂₃N₅O [M+H]⁺: 326.1982; found 326.1981.

### Methyl 1-(2-aminopyrimidin-4-yl)-3-(3-phenylpropyl)piperidine-3-carboxylate (PLH1209)

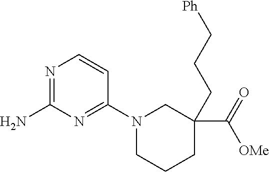 [SMILES: COC(=O)C1(CCCc2ccccc2)CCCN(c2ccnc(N)n2)C1]

The title compound was prepared according to General Procedure 1 from methyl 3-(3-phenylpropyl)piperidine-3-carboxylate (130.1 mg, 0.50 mmol) and 2-amino-4-chloropyridine (70.9 mg, 0.55 mmol) using K₂CO₃ (108.1 mg, 0.78 mmol) in DMSO (1.5 mL) at 120° C. for 22.4 h and was purified by flash column chromatography on silica (25% to 0% Hexane/Acetone) to give a pale yellow oil (112.2 mg, 63%). ¹H NMR (600 MHz, CDCl₃): δ 7.80 (d, J=6.2 Hz, 1H), 7.28-7.20 (m, 2H), 7.16 (tt, J=7.5, 1.3 Hz, 1H), 7.11 (d, J=6.7 Hz, 2H), 5.98 (d, J=6.3 Hz, 1H), 4.93 (s, 2H), 4.06 (d, J=13.8 Hz, 1H), 3.95 (s, 1H), 3.59 (s, 3H), 3.16 (d, J=13.4 Hz, 1H), 3.08 (ddd, J=13.1, 9.2, 3.8 Hz, 1H), 2.53 (t, J=7.2 Hz, 2H), 2.19-2.13 (m, 1H), 1.57 (dddd, J=22.6, 20.4, 9.6, 5.0 Hz, 3H), 1.51-1.42 (m, 2H); ¹³C NMR (151 MHz, CDCl₃): δ 175.32, 162.71, 162.66, 156.42, 141.86, 128.45, 128.42, 125.99, 94.56, 51.78, 50.78, 47.02, 43.88, 36.23, 36.14, 32.48, 26.03, 22.27; HRMS (ESI/LC-Q-TOF): m/z cald for C₂₀H₂₆N₄O₂[M+H]⁺: 355.2135; found 355.2131.

### 4-(3-((Benzyloxy)methyl)-3-(3-phenylpropyl)piperidin-1-yl)pyrimidin-2-amine (PLH1253)

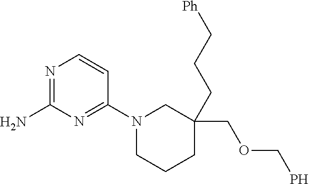

The title compound was prepared according to General Procedure 1 from 3-((benzyloxy)methyl)-3-(3-phenylpropyl)piperidine (125.2 mg, 0.39 mmol) and 2-amino-4-chloropyridine (57.1 mg, 0.44 mmol) using K₂CO₃ (81.6 mg, 0.59 mmol) in DMSO (1.0 mL) at 120° C. for 16.5 h and was purified by flash column chromatography on silica (10% to 5% Hexane/Acetone) to give a clear oil (109.6 mg, 68%). ¹H NMR (600 MHz, CDCl₃): δ 7.76 (d, J=6.2 Hz, 1H), 7.33 (dd, J=8.6, 6.5 Hz, 2H), 7.29-7.25 (m, 5H), 7.20-7.12 (m, 3H), 5.95 (d, J=6.3 Hz, 1H), 4.78 (s, 2H), 4.41 (q, J=12.1 Hz, 2H), 3.81 (s, 1H), 3.62 (d, J=13.3 Hz, 1H), 3.32 (ddd, J=12.7, 7.9, 4.3 Hz, 1H), 3.26-3.12 (m, 3H), 2.56 (t, J=7.2 Hz, 2H), 1.65-1.47 (m, 6H), 1.42 (ddd, J=13.2, 8.3, 5.1 Hz, 1H), 1.36-1.28 (m, 1H); ¹³C NMR (151 MHz, CDCl₃): δ 162.71, 162.58, 156.19, 142.54, 138.65, 128.38, 128.32, 128.29, 127.49, 127.44, 125.72, 94.25, 73.18, 72.42, 50.83, 44.11, 38.15, 36.67, 34.38, 31.44, 25.20, 21.24; HRMS (ESI/LC-Q-TOF): m/z calcd for C₂₆H₃₂N₄O [M+H]⁺: 417.2656; found 417.2658.

### 4-(3-((Benzyloxy)methyl)piperidin-1-yl)pyrimidin-2-amine (PLH2027)

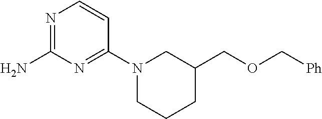 [SMILES: Nc1nccc(N2CCCC(COCc3ccccc3)C2)n1]

The title compound was prepared according to General Procedure 1 from 3-((benzyloxy)methyl)piperidine (206.3 mg, 1.00 mmol) and 2-amino-4-chloropyrimidine (142.6 mg, 1.10 mmol) using K₂CO₃ (207.5 mg, 1.50 mmol) in DMSO (2.0 mL) at 120° C. for 19.9 h and was purified by flash column chromatography on silica (10% to 0% Hexane/EtOAc) to give an off-white solid (218.5 mg, 73%). ¹H NMR (600 MHz, CDCl₃): δ 7.80 (d, J=6.2 Hz, 1H), 7.35-7.30 (m, 4H), 7.29-7.25 (m, 1H), 5.92 (d, J=6.2 Hz, 1H), 5.01 (s, 2H), 4.51-4.45 (m, 2H), 4.13 (dd, J=66.6, 12.1 Hz, 2H), 3.37-3.29 (m, 2H), 2.96 (ddd, J=13.1, 10.9, 3.2 Hz, 1H), 2.79 (dd, J=13.2, 9.8 Hz, 1H), 1.87 (dtt, J=11.3, 9.7, 4.6 Hz, 1H), 1.80 (dq, J=12.9, 4.2 Hz, 1H), 1.69-1.63 (m, 1H), 1.46 (dtt, J=13.3, 11.0, 4.0 Hz, 1H), 1.29 (dddd, J=13.0, 11.2, 10.2, 4.0 Hz, 1H); ¹³C NMR (151 MHz, CDCl₃): δ 162.84, 162.55, 156.43, 138.39, 128.37, 127.57, 127.54, 94.16, 73.04, 72.59, 47.41, 44.49, 36.10, 27.69, 24.23; HRMS (ESI/LC-Q-TOF): m/z calcd for C₁₇H₂₂N₄O [M+H]⁺: 299.1873; found 299.1872.

### (S)-4-(3-((Benzyloxy)methyl)piperidin-1-yl)pyrimidin-2-amine (PLH2032)

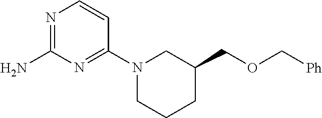 [SMILES: Nc1nccc(N2CCC[C@H](COCc3ccccc3)C2)n1]

The title compound was prepared according to General Procedure 1 from (S)-3-((benzyloxy)methyl)piperidine (267.2 mg, 1.30 mmol) and 2-amino-4-chloropyrimidine (188.0 mg, 1.45 mmol) using K₂CO₃ (272.0 mg, 1.97 mmol) in DMSO (2.5 mL) at 120° C. for 15.2 h and was purified by flash column chromatography on silica (10% to 0% Hexane/EtOAc) to give an off-white solid (312.0 mg, 80%). ¹H NMR (600 MHz, CDCl₃): δ 7.79 (d, J=6.2 Hz, 1H), 7.33-7.28 (m, 4H), 7.25 (ddd, J=8.4, 3.6, 2.5 Hz, 1H), 5.90 (d, J=6.2 Hz, 1H), 5.09 (s, 2H), 4.47 (d, J=2.8 Hz, 2H), 4.12 (dd, J=70.1, 13.8 Hz, 2H), 3.35-3.27 (m, 2H), 2.94 (ddd, J=13.1, 10.9, 3.2 Hz, 1H), 2.77 (dd, J=13.2, 9.8 Hz, 1H), 1.89-1.75 (m, 2H), 1.67-1.61 (m, 1H), 1.45 (dddd, J=15.1, 9.2, 7.6, 4.0 Hz, 1H), 1.27 (dtd, J=13.2, 10.6, 4.0 Hz, 1H); ¹³C NMR (151 MHz, CDCl₃): δ 162.84, 162.54, 156.33, 138.40, 128.36, 127.55, 127.53, 94.11, 73.03, 72.59, 47.41, 44.50, 36.10, 27.68, 24.22; HRMS (ESI/LC-Q-TOF): m/z calcd for C₁₇H₂₂N₄O [M+H]⁺: 299.1873; found 299.1880.

### (R)-4-(3-((Benzyloxy)methyl)piperidin-1-yl)pyrimidin-2-amine (PLH2040)

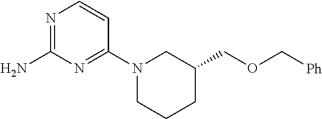 [SMILES: Nc1nccc(N2CCC[C@@H](COCc3ccccc3)C2)n1]

The title compound was prepared according to General Procedure 1 from (R)-3-((benzyloxy)methyl)piperidine (585.4 mg, 2.85 mmol) and 2-amino-4-chloropyrimidine (409.1 mg, 3.16 mmol) using K₂CO₃ (591.7 mg, 4.28 mmol) in DMSO (6 mL) at 120° C. for 18.3 h and was purified by flash column chromatography on silica (10% to 0% Hexane/EtOAc) to give an pale white solid (610.7 mg, 72%). ¹H NMR (600 MHz, CDCl₃): δ 7.80 (dd, J=6.2, 1.4 Hz, 1H), 7.37-7.30 (m, 3H), 7.30-7.23 (m, 1H), 5.94 (dd, J=6.3, 1.4 Hz, 1H), 4.83 (s, 2H), 4.52-4.44 (m, 2H), 4.13 (dd, J=60.8, 12.6 Hz, 2H), 3.38-3.28 (m, 2H), 2.98 (t, J=10.8 Hz, 1H), 2.81 (dd, J=12.6, 10.3 Hz, 1H), 1.92-1.77 (m, 2H), 1.67 (dt, J=13.4, 4.2 Hz, 1H), 1.52-1.43 (m, 1H), 1.31 (qd, J=15.7, 11.3, 6.9, 2.7 Hz, 1H); ¹³C NMR (151 MHz, CDCl₃): δ 162.64, 162.54, 156.26, 138.38, 128.37, 127.58, 127.53, 94.28, 73.07, 72.58, 47.41, 44.50, 36.12, 27.69, 24.22; HRMS (ESI/LC-Q-TOF): m/z calcd for C₁₇H₂₂N₄O [M+H]⁺: 299.1873; found 299.1870.

### 4-(3-((Benzyloxy)methyl)piperidin-1-yl)quinazoline (PLH2069)

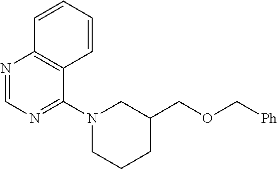 [SMILES: c1ccc(COCC2CCCN(c3ncnc4ccccc34)C2)cc1]

The title compound was prepared according to General Procedure 1 from 3-((benzyloxy)methyl)piperidine (107.1 mg, 0.522 mmol) and 4-chloroquinazoline (82.8 mg, 0.503 mmol) using K₂CO₃ (136.5 mg, 0.988 mmol) in DMSO (1.5 mL) at 80° C. for 2.3 h and was purified by flash column chromatography on deactivated silica (25% EtOAc/Hexane) to give an off-white solid (150.9 mg, 90%). ¹H NMR (800 MHz, CDCl₃): δ 8.81 (s, 1H), 7.99 (dd, J=8.5, 1.2 Hz, 1H), 7.95 (dd, J=8.3, 1.2 Hz, 1H), 7.79 (ddd, J=8.4, 6.9, 1.4 Hz, 1H), 7.48 (ddd, J=8.2, 6.9, 1.2 Hz, 1H), 7.42-7.37 (m, 4H), 7.37-7.34 (m, 1H), 4.58 (q, J=29.9, 12.0 Hz, 2H), 4.43 (d, J=13.0 Hz, 1H), 4.27 (d, J=13.1 Hz, 1H), 3.55-3.48 (m, 2H), 3.29 (ddd, J=13.0, 11.0, 3.0 Hz, 1H), 3.15 (dd, J=13.0, 10.0 Hz, 1H), 2.31-2.25 (m, 1H), 2.03 (dq, J=12.7, 4.1 Hz, 1H), 1.95 (dqd, J=11.6, 4.2, 2.9 Hz, 1H), 1.87 (dtt, J=13.4, 11.3, 4.1 Hz, 1H), 1.49 (dtd, J=13.1, 11.1, 4.2 Hz, 1H); ¹³C NMR (201 MHz, CDCl₃): δ 164.89, 154.13, 151.76, 138.33, 132.30, 128.49, 128.36, 127.57, 127.48, 125.20, 125.17, 116.83, 73.10, 72.59, 53.68, 50.60, 36.87, 27.79, 24.79; HRMS (ESI/LC-Q-TOF): m/z calcd for C₂₁H₂₃N₃O [M+H]⁺: 334.1914; found 334.1918.

### 1-(3-((Benzyloxy)methyl)piperidin-1-yl)isoquinoline (MLKUVA36)

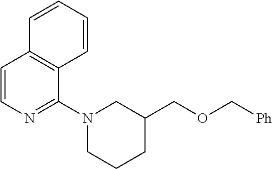 [SMILES: c1ccc(COCC2CCCN(c3nccc4ccccc34)C2)cc1]

The title compound was prepared according to General Procedure 1 from 3-((benzyloxy)methyl)piperidine (106 mg, 0.516 mmol) and 1-chloroisoquinoline (91 mg, 0.56 mmol) using K₂CO₃ (181 mg, 1.31 mmol) in DMSO (2.0 mL) at 80° C. for 18 h and purified by flash column chromatography on silica (10% EtOAc:hexane) to give a yellow oil (37 mg, 22%). ¹H NMR (600 MHz; CDCl₃): δ 8.15 (d, J=5.7 Hz, 1H), 8.11 (d, J=8.4 Hz, 1H), 7.73 (d, J=8.1 Hz, 1H), 7.60 (ddd, J=8.1, 6.8, 1.2 Hz, 1H), 7.48 (ddd, J=8.2, 6.8, 1.3 Hz, 1H), 7.34-7.30 (m, 4H), 7.28-7.25 (m, 1H), 7.22 (d, J=5.7 Hz, 1H), 4.53 (s, 2H), 3.87 (d, J=12.2 Hz, 1H), 3.70 (d, J=12.4 Hz, 1H), 3.48 (d, J=6.2 Hz, 2H), 3.00 (dt, J=13.0, 6.8 Hz, 1H), 2.89-2.83 (m, 1H), 2.29 (ttt, J=10.3, 7.0, 3.8 Hz, 1H), 1.97 (dq, J=12.8, 4.2 Hz, 1H), 1.88 (tt, J=8.5, 3.9 Hz, 2H), 1.36-1.26 (m, 1H); ¹³C NMR (151 MHz, CDCl₃): δ 162.3, 140.7, 138.7, 138.2, 129.7, 128.4, 127.59, 127.57, 127.1, 126.1, 125.9, 122.2, 115.7, 73.4, 73.2, 55.5, 52.8, 37.1, 28.1, 25.2; HRMS (ESI/LC-Q-TOF): m/z calcd for C₂₂H₂₄N₂O [M+H]⁺: 333.1961; found: 333.1968.

### (1-(4,6-Dimethoxy-1,3,5-triazin-2-yl)-3-(3-phenylpropyl)piperidin-3-yl)methanol (PLH1230)

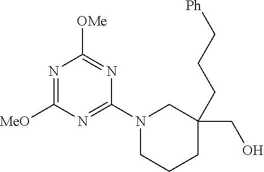 [SMILES: COc1nc(OC)nc(N2CCCC(CO)(CCCc3ccccc3)C2)n1]

The title compound was prepared according to General Procedure 1 from (3-(3-phenylpropyl)piperidin-3-yl)methanol (117.4 mg, 0.503 mmol) and 2-chloro-4,6-dimethoxytriazine (97.7 mg, 0.556 mmol) using K₂CO₃ (103.7 mg, 0.75 mmol) in DMSO (1.0 mL) at 120° C. for 14.0 h and was purified by flash column chromatography on silica (50% Hexane/Acetone) to give a pale yellow oil (693.4 mg, 55%). ¹H NMR (600 MHz, CDCl₃): δ 7.30-7.24 (m, 2H), 7.18 (ddt, J=8.6, 3.7, 1.6 Hz, 3H), 4.53 (d, J=13.0 Hz, 1H), 4.28 (dt, J=13.7, 1.7 Hz, 1H), 3.95 (s, 3H), 3.93 (s, 3H), 3.43 (d, J=12.0 Hz, 1H), 3.24 (d, J=12.0 Hz, 1H), 3.03 (ddd, J=13.2, 10.4, 4.0 Hz, 1H), 2.84 (d, J=13.6 Hz, 1H), 2.67-2.52 (m, 2H), 1.73-1.65 (m, 1H), 1.65-1.50 (m, 4H), 1.48-1.39 (m, 2H), 1.28-1.20 (m, 2H); ¹³C NMR (151 MHz, CDCl₃): δ 172.47, 166.44, 142.55, 128.51, 128.44, 125.89, 62.95, 54.75, 50.19, 45.33, 39.14, 36.83, 35.45, 31.11, 25.16, 21.69; HRMS (ESI/LC-Q-TOF): m/z calcd for C₂₀H₂₈N₄O₃ [M+H]⁺: 373.2241; found 373.2242.

### (1-(4-Amino-1,3,5-triazin-2-yl)-3-(3-phenylpropyl)piperidin-3-yl)methanol (PLH1239)

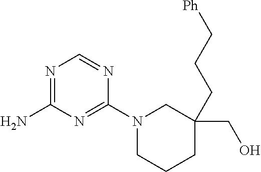 [SMILES: Nc1ncnc(N2CCCC(CO)(CCCc3ccccc3)C2)n1]

The title compound was prepared according to General Procedure 1 from (3-(3-phenylpropyl)piperidin-3-yl)methanol (117.4 mg, 0.503 mmol) and the previously prepared 4-chloro-1,3,5-triazin-2-amine, using K₂CO₃ (104.6 mg, 0.757 mmol) in DMSO (1.0 mL) at 25° C. for 19.0 h and was purified by flash column chromatography on silica (25% Hexane/Acetone) to give white crystals (20.7 mg, 13%). ¹H NMR (600 MHz, CDCl₃): δ 8.12 (d, J=14.2 Hz, 1H), 7.33-7.24 (m, 2H), 7.18 (d, J=7.5 Hz, 3H), 5.36 (s, 2H), 4.55 (dd, J=25.7, 13.1 Hz, 1H), 4.28 (dd, J=57.4, 13.7 Hz, 1H), 3.42 (t, J=13.5 Hz, 1H), 3.22 (dd, J=16.5, 11.9 Hz, 1H), 2.91 (dt, J=58.0, 12.3 Hz, 1H), 2.72 (d, J=26.9 Hz, 1H), 2.59 (dddd, J=20.3, 14.3, 10.7, 5.7 Hz, 2H), 1.68 (ddt, J=14.5, 8.7, 4.3 Hz, 1H), 1.65-1.47 (m, 5H), 1.42 (dq, J=13.9, 9.8, 6.9 Hz, 1H), 1.27-1.16 (m, 1H); ¹³C NMR (151 MHz, 50° C., CDCl₃): δ 166.28, 164.23, 142.64, 128.56, 128.48, 125.93, 125.65, 39.15, 36.94, 35.86, 34.44, 31.35, 30.58, 25.14, 21.77, 21.32; HRMS (ESI/LC-Q-TOF): m/z calcd for C₁₈H₂₅N₅O [M+H]⁺: 328.2139; found 328.2136.

### (1-(4,6-Diamino-1,3,5-triazin-2-yl)-3-(3-phenylpropyl)piperidin-3-yl)methanol (PLH1258)

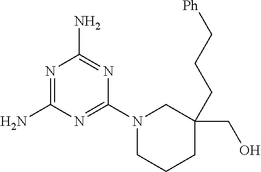 [SMILES: Nc1nc(N)nc(N2CCCC(CO)(CCCc3ccccc3)C2)n1]

The title compound was prepared according to General Procedure 1 from (3-(3-phenylpropyl)piperidin-3-yl)methanol (236.6 mg, 1.01 mmol) and 6-chloro-1,3,5-triazine-2,4-diamine (170.0, 1.17 mmol) using K₂CO₃ (210.7 mg, 1.52 mmol) in DMSO (2.0 mL) at 120° C. for 21.5 h and was purified by flash column chromatography on silica (5% to 10% MeOH/DCM) followed by alumina (10% MeOH/DCM) to give a white oily solid (119.8 mg, 35%). ¹H NMR (598 MHz, Methanol-d4) δ 7.23 (t, J=7.6 Hz, 2H), 7.17-7.10 (m, 3H), 4.00 (dt, J=13.2, 4.8 Hz, 1H), 3.82 (d, J=13.4 Hz, 1H), 3.39 (d, J=11.7 Hz, 1H), 3.34-3.29 (m, 1H), 3.27 (d, J=11.7 Hz, 1H), 3.15 (d, J=13.4 Hz, 1H), 2.55 (ddd, J=8.5, 6.9, 1.9 Hz, 2H), 1.66 (dddt, J=12.6, 8.3, 6.8, 4.5 Hz, 1H), 1.62-1.34 (m, 6H), 1.24 (td, J=13.2, 4.4 Hz, 1H); ¹³C NMR (150 MHz, Methanol-d4) δ 168.33, 166.40, 143.68, 129.36, 129.18, 126.59, 64.63, 50.53, 45.21, 39.58, 37.69, 35.34, 32.05, 26.11, 22.24; HRMS (ESI/LC-Q-TOF): m/z calcd for C₁₈H₂₆N₆O [M+H]⁺: 343.2248; found 343.2255.

### (1-(2-Amino-5,6-dimethylpyrimidin-4-yl)-3-(3-phenylpropyl)piperidin-3-yl)methanol (PLH1252)

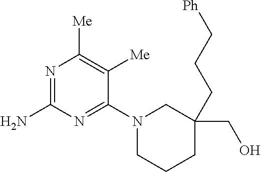 [SMILES: Cc1nc(N)nc(N2CCCC(CO)(CCCc3ccccc3)C2)c1C]

The title compound was prepared according to General Procedure 1 from (3-(3-phenylpropyl)piperidin-3-yl)methanol (233.5 mg, 1.00 mmol) and 2-amino-4-chloro-5,6-dimethylpyrimidine (173.4 mg, 1.10 mmol) using K₂CO₃ (212.2 mg, 1.54 mmol) in DMSO (2.0 mL) at 150° C. for 24.2 h and was purified by flash column chromatography on silica (10% to 0% Hexane/Acetone) followed by alumina (2% to 10% MeOH/EtOAc) to give white crystals (122.5 mg, 35%). ¹H NMR (600 MHz, CDCl₃): δ 7.27 (dd, J=8.1, 6.9 Hz, 2H), 7.20-7.14 (m, 3H), 5.33 (s, 1H), 4.69 (s, 2H), 3.91 (d, J=13.2 Hz, 1H), 3.63-3.56 (m, 2H), 3.28 (d, J=11.7 Hz, 1H), 3.07 (ddd, J=12.8, 11.4, 3.3 Hz, 1H), 2.67-2.53 (m, 3H), 2.21 (s, 3H), 1.97 (s, 3H), 1.72-1.43 (m, 6H), 1.32 (td, J=13.2, 12.0, 4.7 Hz, 1H), 1.26-1.16 (m, 1H); ¹³C NMR (151 MHz, CDCl₃): δ 166.40, 165.43, 159.43, 142.63, 128.39, 128.28, 125.68, 104.22, 62.74, 53.44, 49.48, 37.86, 36.81, 35.96, 30.72, 25.05, 22.63, 22.42, 15.13; HRMS (ESI/LC-Q-TOF): m/z calcd for C₂₁H₃₀N₄O [M+H]⁺: 355.2499; found 355.2505.

### (1-(3-Methylpyrazin-2-yl)-3-(3-phenylpropyl)piperidin-3-yl)methanol (PLH1234)

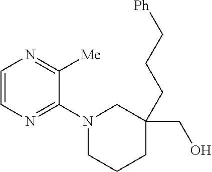 [SMILES: Cc1nccnc1N1CCCC(CO)(CCCc2ccccc2)C1]

The title compound was prepared according to General Procedure 1 from (3-(3-phenylpropyl)piperidin-3-yl)methanol (116.8 mg, 0.50 mmol) and 2-chloro-3-methylpyrazine (57.3 μL, 0.55 mmol) using K₂CO₃ (103.7 mg, 0.75 mmol) in DMSO (1.0 mL) at 120° C. for 26.0 h and was purified by flash column chromatography on silica (50% Hexane/Acetone) followed by alumina (50% to 10% Hexane/EtOAc) to give a clear oil (27.6 mg, 17%). ¹H NMR (600 MHz, CDCl₃): δ 7.94 (d, J=2.9 Hz, 2H), 7.31-7.24 (m, 2H), 7.18 (dd, J=7.4, 2.8 Hz, 3H), 3.66-3.56 (m, 2H), 3.45 (t, J=11.8 Hz, 2H), 3.06 (ddd, J=12.9, 10.5, 3.0 Hz, 1H), 2.79 (dd, J=13.3, 3.1 Hz, 1H), 2.61 (qdd, J=14.0, 9.6, 6.6 Hz, 2H), 2.50 (s, 3H), 1.70-1.49 (m, 6H), 1.43-1.27 (m, 2H); ¹³C NMR (151 MHz, CDCl₃): δ 157.26, 145.91, 142.51, 138.46, 135.16, 128.39, 128.30, 125.74, 64.55, 54.78, 50.10, 37.82, 36.75, 35.40, 30.74, 25.01, 22.76, 22.27; HRMS (ESI/LC-Q-TOF): m/z calcd for C₂₀H₂₇N₃O [M+H]⁺: 326.2234; found 326.2228.

### (3-(3-Phenylpropyl)-1-(6-(trifluoromethyl)pyrimidin-4-yl)piperidin-3-yl)methanol (PLH1259)

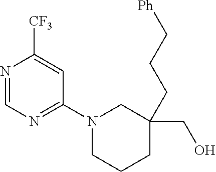 [SMILES: OCC1(CCCc2ccccc2)CCCN(c2cc(C(F)(F)F)ncn2)C1]

The title compound was prepared according to General Procedure 1 from (3-(3-phenylpropyl)piperidin-3-yl)methanol (235.2 mg, 1.01 mmol) and 4-chloro-6-(trifluoromethyl)pyrimidine (140 μL, 1.1 mmol) using K₂CO₃ (207.7 mg, 1.50 mmol) in DMSO (2.0 mL) at 120° C. for 17.4 h and was purified by flash column chromatography on silica (25% to 33% EtOAc/Hexane) followed by alumina (67% to 0% Hexane/EtOAc) to give a thick yellow oil (170.0 mg, 45%). ¹H NMR (600 MHz, CDCl₃): δ 8.66 (s, 1H), 7.35 (t, J=7.7 Hz, 2H), 7.26 (t, J=7.9 Hz, 3H), 6.92 (s, 1H), 4.62-3.69 (m, 2H), 3.65-3.15 (m, 3H), 3.08 (s, 1H), 2.69 (dddd, J=28.7, 14.0, 7.6, 4.6 Hz, 2H), 1.82-1.52 (m, 7H), 1.35 (t, J=11.1 Hz, 1H); ¹³C NMR (151 MHz, 50° C., CDCl₃): δ 161.94, 159.17, 154.73 (q, J=34.6 Hz), 142.33, 128.47, 128.45, 121.20 (q, J=274.8 Hz), 99.13 (q, J=3.4 Hz), 63.81, 50.41, 45.92, 39.15, 36.71, 34.73, 30.85, 25.01, 21.62; ¹⁹F NMR (564 MHz, CDCl₃) δ −70.73; HRMS (ESI/LC-Q-TOF): m/z calcd for C₂₀H₂₄F₃N₃O [M+H]⁺: 380.1951; found 380.1970.

### (1-(2-(Dimethylamino)-5,6-dimethylpyrimidin-4-yl)-3-(3-phenylpropyl)piperidin-3-yl)methanol (PLH1276)

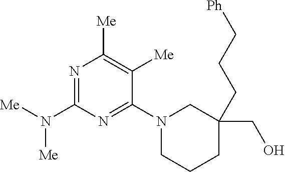 [SMILES: Cc1nc(N(C)C)nc(N2CCCC(CO)(CCCc3ccccc3)C2)c1C]

The title compound was prepared according to General Procedure 1 from (3-(3-phenylpropyl)piperidin-3-yl)methanol (235.8 mg, 1.01 mmol) and 4-chloro-N,N,5,6-tetramethylpyrimidin-2-amine (205.6 mg, 1.11 mmol) using K₂CO₃ (207.4 mg, 1.50 mmol) in DMSO (2.0 mL) at 150° C. for 21.6 h and was purified by flash column chromatography on silica (10% to 50% to 80% EtOAc/Hexane) to give a yellow oil (231.0 mg, 60%). ¹H NMR (600 MHz, CDCl₃): δ 7.30-7.26 (m, 2H), 7.20-7.16 (m, 3H), 3.80 (d, J=13.3 Hz, 1H), 3.63 (d, J=11.4 Hz, 1H), 3.53 (dt, J=12.9, 4.1 Hz, 1H), 3.39 (d, J=11.4 Hz, 1H), 3.11 (s, 6H), 3.04 (ddd, J=12.7, 10.9, 3.2 Hz, 1H), 2.74 (d, J=13.4 Hz, 1H), 2.60 (dddd, J=29.1, 13.7, 8.9, 6.2 Hz, 2H), 2.25 (s, 3H), 1.99 (s, 3H), 1.71-1.58 (m, 3H), 1.54 (dddd, J=16.7, 13.6, 6.5, 3.7 Hz, 3H), 1.37-1.26 (m, 2H); ¹³C NMR (151 MHz, CDCl₃): δ 166.01, 165.63, 160.02, 142.60, 128.37, 128.27, 125.68, 102.02, 64.35, 54.26, 49.69, 37.87, 37.03, 36.77, 35.60, 30.93, 25.05, 22.88, 22.22, 14.76; HRMS (ESI/LC-Q-TOF): m/z calcd for C₂₃H₃₄N₄O [M+H]⁺: 383.2812; found 383.2830.

### (1-(6-Chloro-5-methylpyrimidin-4-yl)-3-(3-phenylpropyl)piperidin-3-yl)methanol (PLH1279)

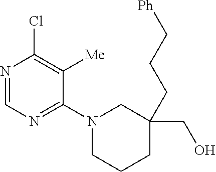 [SMILES: Cc1c(Cl)ncnc1N1CCCC(CO)(CCCc2ccccc2)C1]

The title compound was prepared according to General Procedure 1 from (3-(3-phenylpropyl)piperidin-3-yl)methanol (234.2 mg, 1.00 mmol) and 4,6-dichloro-5-methylpyrimidine (181.7 mg, 1.11 mmol) using K₂CO₃ (207.9 mg, 1.50 mmol) in DMSO (2.0 mL) at 120° C. for 19.2 h and was purified by flash column chromatography on silica (25% to 40% EtOAc/Hexane) to give a pale yellow oil (291.8 mg, 81%). ¹H NMR (600 MHz, CDCl₃): δ 8.21 (s, 1H), 7.24 (td, J=7.2, 1.5 Hz, 2H), 7.16-7.12 (m, 3H), 4.42 (s, 1H), 3.92 (d, J=13.6 Hz, 1H), 3.58 (d, J=12.9 Hz, 1H), 3.52 (d, J=11.7 Hz, 1H), 3.28 (d, J=11.7 Hz, 1H), 3.19-3.12 (m, 1H), 2.79 (d, J=13.8 Hz, 1H), 2.63-2.51 (m, 2H), 2.17 (s, 3H), 1.69-1.48 (m, 6H), 1.40-1.32 (m, 1H), 1.25-1.16 (m, 1H); ¹³C NMR (151 MHz, CDCl₃) δ 165.39, 161.07, 154.01, 142.40, 128.34, 128.25, 125.69, 114.57, 62.79, 53.88, 49.53, 38.18, 36.65, 35.19, 30.44, 24.86, 22.40, 16.99; HRMS (ESI/LC-Q-TOF): m/z calcd for C₂₀H₂₆ClN₃O [M+H]⁺: 360.1844; found 360.1860.

### (3-(3-Phenylpropyl)-1-(quinazolin-4-yl)piperidin-3-yl)methanol (PLH1295)

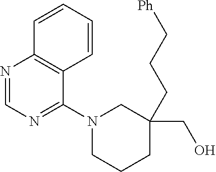 [SMILES: OCC1(CCCc2ccccc2)CCCN(c2ncnc3ccccc23)C1]

The title compound was prepared according to General Procedure 1 from (3-(3-phenylpropyl)piperidin-3-yl)methanol (234.8 mg, 1.01 mmol) and 4-chloroquinazoline (180.9 mg, 1.10 mmol) using K₂CO₃ (207.4 mg, 1.50 mmol) in DMSO (2.0 mL) at 80° C. for 1.6 h and was purified by flash column chromatography on silica (33% Acetone/Hexane) to give a white, foamy solid (75.1 mg, 21%). ¹H NMR (600 MHz, CDCl₃): δ 8.56 (s, 1H), 7.84 (ddd, J=14.2, 8.4, 1.3 Hz, 2H), 7.68 (ddd, J=8.3, 6.9, 1.3 Hz, 1H), 7.39 (ddd, J=8.3, 6.9, 1.3 Hz, 1H), 7.26 (t, J=7.6 Hz, 2H), 7.21-7.13 (m, 3H), 5.30 (s, 1H), 4.34 (d, J=13.7 Hz, 1H), 4.24 (d, J=13.0 Hz, 1H), 3.60 (d, J=11.9 Hz, 1H), 3.54 (ddd, J=12.9, 11.1, 4.1 Hz, 1H), 3.31 (d, J=11.9 Hz, 1H), 2.94 (d, J=13.7 Hz, 1H), 2.69-2.55 (m, 2H), 1.76-1.50 (m, 6H), 1.49-1.41 (m, 1H), 1.29-1.22 (m, 1H); ¹³C NMR (151 MHz, CDCl₃): δ 163.55, 153.34, 152.03, 142.48, 132.68, 128.46, 128.38, 128.28, 125.71, 125.44, 125.01, 116.13, 62.30, 54.04, 51.73, 38.38, 36.69, 35.70, 30.68, 24.90, 22.99; HRMS (ESI/LC-Q-TOF): m/z calcd for C₂₃H₂₇N₃O [M+H]⁺: 362.2234; found 362.2241.

### (3-(3-Phenylpropyl)-1-(pyridin-2-VI)piperidin-3-VI)methanol (PLH1291)

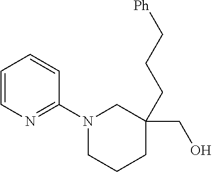 [SMILES: OCC1(CCCc2ccccc2)CCCN(c2ccccn2)C1]

The title compound was prepared according to General Procedure 1 from (3-(3-phenylpropyl)piperidin-3-yl)methanol (235.5 mg, 1.01 mmol) and 2-chloropyridine (104 μL, 1.10 mmol) using K₂CO₃ (207.6 mg, 1.50 mmol) in DMSO (2.0 mL) at 120° C. for 25.8 h then 140° C. for 15.3 h and was purified by flash column chromatography on silica (50% Acetone/Hexane) to give a yellow oil (95.9 mg, 31%). ¹H NMR (600 MHz, CDCl₃): δ 8.03 (d, J=5.1 Hz, 1H), 7.41 (ddd, J=8.7, 6.8, 1.9 Hz, 1H), 7.29 (d, J=7.5 Hz, 2H), 7.24-7.13 (m, 3H), 6.61 (d, J=8.8 Hz, 1H), 6.52-6.45 (m, 1H), 4.73 (s, 1H), 4.40 (dt, J=13.9, 2.2 Hz, 1H), 3.87 (d, J=14.3 Hz, 1H), 3.45 (d, J=11.8 Hz, 1H), 3.22 (d, J=11.8 Hz, 1H), 3.04 (ddd, J=13.8, 11.9, 3.3 Hz, 1H), 2.71-2.51 (m, 3H), 1.77-1.67 (m, 1H), 1.66-1.46 (m, 5H), 1.42 (td, J=12.9, 4.5 Hz, 1H), 1.23 (dd, J=12.5, 9.2 Hz, 1H); ¹³C NMR (151 MHz, CDCl₃): δ 158.49, 147.64, 142.70, 137.84, 128.41, 128.27, 125.67, 111.63, 106.69, 62.54, 50.50, 46.91, 36.84, 36.02, 31.32, 25.08, 21.69; HRMS (ESI/LC-Q-TOF): m/z calcd for C₂₀H₂₆N₂O [M+H]⁺: 311.2125; found 311.2135.

### (3-(3-Phenylpropyl)-1-(pyrimidin-4-VI)piperidin-3-yl)methanol (PLH2062)

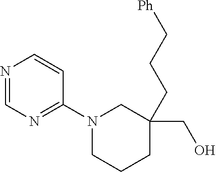 [SMILES: OCC1(CCCc2ccccc2)CCCN(c2ccncn2)C1]

The title compound was prepared according to General Procedure 1 from (3-(3-phenylpropyl)piperidin-3-yl)methanol (235.4 mg, 1.01 mmol) and 4-chloropyrimidine (127.8 mg, 1.12 mmol) using K₂CO₃ (210.4 mg, 1.52 mmol) in DMSO (2.0 mL) at 120° C. for 15.3 h and was purified by flash column chromatography on silica (0% to 10% MeOH/EtOAc) to give a yellow oil (44.0 mg, 14%). ¹H NMR (600 MHz, CDCl₃): δ 8.49 (d, J=1.2 Hz, 1H), 8.10 (d, J=6.3 Hz, 1H), 7.29-7.24 (m, 2H), 7.20-7.14 (m, 3H), 6.49 (dd, J=6.4, 1.3 Hz, 1H), 4.28 (s, 1H), 3.84 (dd, J=12.6, 4.9 Hz, 1H), 3.39 (d, J=11.8 Hz, 1H), 3.25 (d, J=11.8 Hz, 1H), 3.13 (p, J=5.8, 4.1 Hz, 1H), 2.78 (d, J=13.6 Hz, 1H), 2.67-2.51 (m, 2H), 1.73-1.64 (m, 1H), 1.63-1.38 (m, 7H), 1.29-1.20 (m, 1H); ¹³C NMR (151 MHz, CDCl₃): δ 160.76, 158.32, 155.31, 142.39, 128.34, 128.28, 125.73, 102.92, 62.74, 49.85, 45.71, 38.77, 36.64, 35.16, 30.86, 24.97, 21.57; HRMS (ESI/LC-Q-TOF): m/z calcd for C₁₉H₂₅N₃O [M+H]⁺: 312.2077; found 312.2090.

### (1-(6-Chloro-5-methoxypyrimidin-4-yl)-3-(3-phenylpropyl)piperidin-3-yl)methanol (PLH1302)

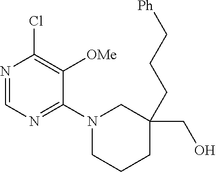 [SMILES: COc1c(Cl)ncnc1N1CCCC(CO)(CCCc2ccccc2)C1]

The title compound was prepared according to General Procedure 1 from (3-(3-phenylpropyl)piperidin-3-yl)methanol (234.7 mg, 1.01 mmol) and 4,6-dichloro-5-methoxypyrimidine (197.2 mg, 1.1 mmol) using K₂CO₃ (207.2, 1.50 mmol) in DMSO (2.0 mL) at 120° C. for 16.5 h and was purified by flash column chromatography on silica (10% to 25% to 50% EtOAc/Hexane) to give a pale yellow oil (114.9 mg, 31%). ¹H NMR (600 MHz, CDCl₃): δ 8.10 (s, 1H), 7.30-7.24 (m, 2H), 7.20-7.15 (m, 3H), 4.72 (s, 1H), 4.52 (d, J=14.1 Hz, 1H), 4.25 (dt, J=13.7, 1.8 Hz, 1H), 3.68 (s, 3H), 3.47 (d, J=12.0 Hz, 1H), 3.30-3.21 (m, 2H), 2.82 (d, J=13.7 Hz, 1H), 2.68-2.51 (m, 2H), 1.75-1.64 (m, 1H), 1.64-1.51 (m, 5H), 1.46-1.37 (m, 1H), 1.20 (dd, J=12.6, 9.3 Hz, 1H); ¹³C NMR (151 MHz, CDCl₃): δ 156.77, 152.98, 151.95, 142.35, 128.36, 128.29, 125.74, 62.51, 59.77, 52.81, 47.31, 38.71, 36.61, 35.32, 30.80, 24.80, 22.65; HRMS (ESI/LC-Q-TOF): m/z calcd for C₂₀H₂₆ClN₃O₂[M+H]⁺: 376.1793; found 376.1807.

### (1-(6-Ethyl-5-fluoropyrimidin-4-yl)-3-(3-phenylpropyl)piperidin-3-yl)methanol (PLH2002)

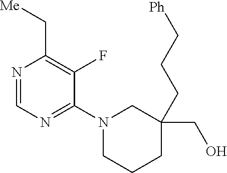 [SMILES: CCc1ncnc(N2CCCC(CO)(CCCc3ccccc3)C2)c1F]

The title compound was prepared according to General Procedure 1 from (3-(3-phenylpropyl)piperidin-3-yl)methanol (234.5 mg, 1.00 mmol) and 4-chloro-6-ethyl-5-fluoropyrimidine (137 μL, 1.1 mmol) using K₂CO₃ (207.8, 1.50 mmol) in DMSO (2.0 mL) at 120° C. for 17.3 h and was purified by flash column chromatography on silica (25% to 50% EtOAc/Hexane) to give a pale yellow oil (289.6 mg, 81%). ¹H NMR (600 MHz, CDCl₃): δ 8.22 (d, J=2.3 Hz, 1H), 7.29-7.24 (m, 2H), 7.20-7.15 (m, 3H), 4.25 (dt, J=13.8, 1.8 Hz, 1H), 4.18 (d, J=13.6 Hz, 1H), 4.11 (dd, J=10.1, 4.2 Hz, 1H), 3.50 (dd, J=11.9, 3.9 Hz, 1H), 3.29-3.19 (m, 2H), 2.76 (d, J=13.7 Hz, 1H), 2.70 (qd, J=7.6, 2.9 Hz, 2H), 2.67-2.53 (m, 2H), 1.74-1.65 (m, 1H), 1.65-1.52 (m, 5H), 1.44-1.36 (m, 1H), 1.25 (t, J=7.6 Hz, 3H); ¹³C NMR (151 MHz, CDCl₃): δ 156.25 (d, J=15.7 Hz), 152.43 (d, J=9.6 Hz), 151.27 (d, J=5.6 Hz), 143.90 (d, J=256.3 Hz), 142.44, 128.35, 128.27, 125.71, 62.55, 52.16, 47.97 (d, J=13.8 Hz), 38.56, 35.40, 30.87, 24.88, 23.94, 22.52, 12.15 (d, J=1.3 Hz); HRMS (ESI/LC-Q-TOF): m/z calcd for C₂₁H₂₈FN₃O [M+H]⁺: 358.2296; found 358.2309.

### (1-(4-Aminopyrimidin-2-yl)-3-(3-phenylpropyl)piperidin-3-yl)methanol (PLH2006)

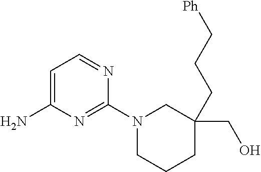 [SMILES: Nc1ccnc(N2CCCC(CO)(CCCc3ccccc3)C2)n1]

The title compound was prepared according to General Procedure 1 from (3-(3-phenylpropyl)piperidin-3-yl)methanol (237.3 mg, 1.02 mmol) and 4-amino-2-chloropyrimidine (146.3 mg, 1.13 mmol) using K₂CO₃ (208.3, 1.51 mmol) in DMSO (2.0 mL) at 120° C. for 22.3 h and was purified by flash column chromatography on silica (33% to 50% Acetone/Hexane) to give an off-white, foamy solid (40.0 mg, 12%). ¹H NMR (600 MHz, CDCl₃): δ 7.82 (dt, J=5.7, 3.1 Hz, 1H), 7.27 (ddd, J=8.5, 6.8, 1.9 Hz, 2H), 7.18 (dd, J=7.5, 5.5 Hz, 3H), 5.66 (dd, J=5.8, 1.7 Hz, 1H), 4.87 (d, J=4.9 Hz, 2H), 4.70-4.59 (m, 1H), 4.35 (ddd, J=13.8, 4.5, 2.3 Hz, 1H), 3.44 (d, J=12.1 Hz, 1H), 3.18 (d, J=12.1 Hz, 1H), 2.87-2.76 (m, 1H), 2.68-2.49 (m, 3H), 1.74-1.34 (m, 6H), 1.26-1.12 (m, 1H); ¹³C NMR (151 MHz, CDCl₃): δ 163.38, 161.30, 142.66, 128.39, 128.26, 125.65, 102.68, 94.12, 62.23, 50.14, 45.10, 38.51, 36.82, 36.19, 31.37, 25.08, 21.64; HRMS (ESI/LC-Q-TOF): m/z calcd for C₁₉H₂₆N₄O [M+H]⁺: 327.2186; found 327.2197.

### (3-(3-Phenylpropyl)-1-(quinolin-4-yl)piperidin-3-yl)methanol (PLH2058)

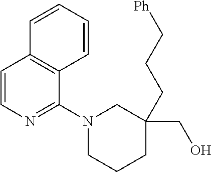 [SMILES: OCC1(CCCc2ccccc2)CCCN(c2nccc3ccccc23)C1]

The title compound was prepared according to General Procedure 1 from (3-(3-phenylpropyl)piperidin-3-yl)methanol (234.1 mg, 1.00 mmol) and 4-chloroquinoline (181.1 mg, 1.11 mmol) using K₂CO₃ (204.9 mg, 1.48 mmol) in DMSO (2.0 mL) at 120° C. for 15.9 h and was purified by flash column chromatography on silica (10% to 20% Acetone/Hexane) to give a pale yellow oil (103.0 mg, 29%). ¹H NMR (600 MHz, CDCl₃): δ 7.99 (t, J=6.7 Hz, 2H), 7.70 (dd, J=8.1, 1.2 Hz, 1H), 7.58 (ddd, J=8.1, 6.8, 1.2 Hz, 1H), 7.45 (ddd, J=8.3, 6.8, 1.3 Hz, 1H), 7.29 (t, J=7.6 Hz, 2H), 7.24-7.16 (m, 3H), 7.10 (dd, J=5.9, 0.7 Hz, 1H), 5.11 (s, 1H), 4.00 (d, J=13.4 Hz, 1H), 3.92 (d, J=12.9 Hz, 1H), 3.80-3.72 (m, 1H), 3.45 (dd, J=11.7, 6.3 Hz, 1H), 3.38 (ddd, J=12.8, 11.5, 3.3 Hz, 1H), 2.95 (d, J=13.4 Hz, 1H), 2.71-2.58 (m, 2H), 1.80-1.57 (m, 5H), 1.53 (dt, J=13.7, 3.8 Hz, 1H), 1.45 (ddd, J=13.5, 12.4, 4.6 Hz, 1H), 1.39-1.29 (m, 1H); ¹³C NMR (151 MHz, CDCl₃): δ 160.63, 142.65, 139.89, 138.83, 129.70, 128.41, 128.26, 127.01, 126.10, 125.65, 125.40, 120.87, 113.94, 63.95, 55.43, 52.93, 37.82, 36.82, 36.14, 31.19, 25.05, 22.78; HRMS (ESI/LC-Q-TOF): m/z calcd for C₂₄H₂₈N₂O [M+H]⁺: 361.2281; found 361.2289.

### (1-(7-Chloroquinazolin-4-yl)-3-(3-phenylpropyl)piperidin-3-yl)methanol (PLH2074)

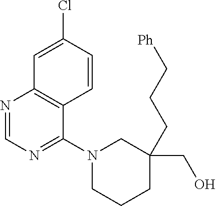 [SMILES: OCC1(CCCc2ccccc2)CCCN(c2ncnc3cc(Cl)ccc23)C1]

The title compound was prepared according General Procedure 1 from (3-(3-phenylpropyl)piperidin-3-yl)methanol (234.3 mg, 1.00 mmol) and 4,7-dichloroquinazoline (219.5 mg, 1.10 mmol) using K₂CO₃ (211.3 mg, 1.53 mmol) in DMSO (2.0 mL) at 80° C. for 2 h and was purified by flash column chromatography on silica (50% to 25% Hexane/EtOAc) to give a pale yellow oil (157.3 mg, 40%). ¹H NMR (600 MHz, CDCl₃): δ 8.54 (s, 1H), 7.81 (d, J=2.2 Hz, 1H), 7.79 (d, J=9.0 Hz, 1H), 7.34 (dd, J=8.9, 2.2 Hz, 1H), 7.30-7.26 (m, 2H), 7.21-7.16 (m, 3H), 4.99 (dd, J=10.1, 4.3 Hz, 1H), 4.36 (dt, J=13.7, 1.9 Hz, 1H), 4.26-4.16 (m, 1H), 3.63-3.50 (m, 2H), 3.35-3.26 (m, 1H), 2.94 (dd, J=13.7, 1.2 Hz, 1H), 2.71-2.54 (m, 2H), 1.78-1.53 (m, 6H), 1.51-1.42 (m, 1H), 1.29-1.22 (m, 1H); ¹³C NMR (151 MHz, CDCl₃): δ 163.27, 154.33, 153.02, 142.40, 138.65, 128.36, 128.28, 127.57, 126.81, 125.73, 125.73, 114.43, 62.27, 54.02, 51.77, 38.43, 36.64, 35.64, 30.61, 24.84, 22.98; HRMS (ESI/LC-Q-TOF): m/z calcd for C₂₃H₂₆ClN₃O [M+H]⁺: 396.1844; found 396.1860.

### (4-(3-Phenylpropyl)-1-(6-(trifluoromethyl)pyrimidin-4-yl)piperidin-4-yl)methanol (JDDUVA47-P1)

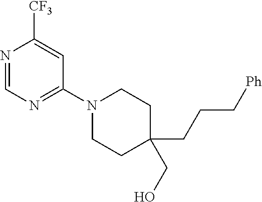 [SMILES: OCC1(CCCc2ccccc2)CCN(c2cc(C(F)(F)F)ncn2)CC1]

The title compound was prepared according to General Procedure 1 from (4-(3-phenylpropyl)piperidin-4-yl)methanol hydrochloride (233 mg, 0.864 mmol) and 4-chloro-6-(trifluoromethyl)pyrimidine (0.14 mL, 1.1 mmol) using K₂CO₃ (345 mg, 2.50 mmol) in DMSO (2.0 mL) at 120° C. for 24 h and was purified by flash column chromatography on alumina (30% EtOAc:hexane then 10%-40% EtOAc:hexane) to give an opaque oil (178 mg, 55%). ¹H NMR (600 MHz, CDCl₃): δ 8.63 (s, 1H), 7.29 (tq, J=7.7, 1.0 Hz, 2H), 7.21-7.17 (m, 3H), 6.76 (s, 1H), 3.75 (br s, 1H), 3.60-3.51 (m, 4H), 2.63 (t, J=7.5 Hz, 2H), 1.65-1.55 (m, 4H), 1.54-1.48 (m, 4H), 1.43 (br s, 1H); ¹³C NMR (151 MHz, CDCl₃): δ 161.6, 158.9, 154.1 (q, J=34.4 Hz), 142.2, 128.43, 128.39, 125.9, 121.1 (q, J=274.7 Hz), 98.9 (q, J=3.4 Hz), 66.9, 40.2, 36.5, 36.0, 33.8, 31.2, 25.1; ¹⁹F NMR (564 MHz; CDCl₃): δ −70.5; HRMS (ESI/LC-Q-TOF): m/z calcd for C₂₀H₂₄F₃N₃O [M+H]⁺: 380.1944; found: 380.1935.

### (1-(2-Amino-5,6-dimethylpyrimidin-4-yl)-4-(3-phenylpropyl)piperidin-4-yl)methanol (JDDUVA43-2BP1)

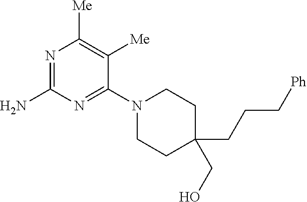 [SMILES: Cc1nc(N)nc(N2CCC(CO)(CCCc3ccccc3)CC2)c1C]

The title compound was prepared according to General Procedure 1 from (4-(3-phenylpropyl)piperidin-4-yl)methanol hydrochloride (117 mg, 0.434 mmol) and 4-chloro-5,6-dimethylpyrimidin-2-amine (87 mg, 0.55 mmol) using K₂CO₃ (104 mg, 0.753 mmol) in DMSO (1.0 mL) at 120° C. for 18 h then 150° C. for 5 h and was purified by flash column chromatography on alumina (0.1% MeOH:EtOAc to 5% MeOH:EtOAc) to give a yellow solid (117 mg, 77%). ¹H NMR (600 MHz, CDCl₃): δ 7.28-7.24 (m, 2H), 7.17 (tq, J=4.9, 1.6 Hz, 3H), 4.97 (s, 2H), 3.78 (br s, 1H), 3.48 (s, 2H), 3.19 (ddd, J=13.1, 7.2, 3.8 Hz, 2H), 3.13 (ddd, J=12.8, 8.0, 3.6 Hz, 2H), 2.58 (t, J=7.6 Hz, 2H), 2.22 (s, 3H), 1.98 (s, 3H), 1.58 (dddd, J=17.5, 10.0, 7.6, 4.5 Hz, 4H), 1.48 (ddd, J=17.6, 8.0, 4.3 Hz, 4H); ¹³C NMR (151 MHz, CDCl₃): δ 167.8, 164.9, 159.9, 142.6, 128.5, 125.9, 106.8, 67.3, 44.7, 36.8, 36.0, 34.2, 32.1, 25.2, 22.0, 14.3; HRMS (ESI/LC-Q-TOF): m/z calcd for C₂₁H₃₀N₄O [M+H]⁺: 355.2492; found: 355.2497.

### (1-(2-Amino-6-methylpyrimidin-4-yl)-4-(3-phenylpropyl)piperidin-4-yl)methanol (JDDUVA44-2BP1)

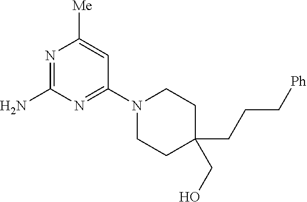 [SMILES: Cc1cc(N2CCC(CO)(CCCc3ccccc3)CC2)nc(N)n1]

The title compound was prepared according to General Procedure 1 from (4-(3-phenylpropyl)piperidin-4-yl)methanol hydrochloride (117 mg, 0.434 mmol) and 4-chloro-6-methylpyrimidin-2-amine (79 mg, 0.55 mmol) using K₂CO₃ (173 mg, 1.25 mmol) in DMSO (1.0 mL) at 150° C. for 18 and was purified by flash column chromatography on alumina (5% MeOH:EtOAc) to give a yellow solid (45 mg, 31%). ¹H NMR (600 MHz, CDCl₃): δ 7.29-7.25 (m, 2H), 7.19-7.16 (m, 3H), 5.79 (s, 1H), 4.85 (s, 2H), 3.63-3.56 (m, 2H), 3.47 (s, 2H), 3.41 (ddd, J=13.0, 8.3, 3.9 Hz, 2H), 2.99 (br s, 1H), 2.60 (t, J=7.6 Hz, 2H), 2.19 (s, 3H), 1.63-1.55 (m, 2H), 1.46 (dddd, J=30.0, 13.5, 7.7, 4.1 Hz, 6H); ¹³C NMR (151 MHz, CDCl₃): δ 165.4, 163.3, 162.4, 142.5, 128.47, 128.46, 126.0, 93.0, 67.0, 39.9, 36.7, 36.1, 34.0, 31.5, 25.2, 23.9; HRMS (ESI/LC-Q-TOF): m/z calcd for C₂₀H₂₈N₄O [M+H]⁺: 341.2336; found: 341.2335.

### 6-(4-(Hydroxymethyl)-4-(3-phenylpropyl)piperidin-1-yl)-5-methylpyrimidin-4-ol (JDDUVA48-P1)

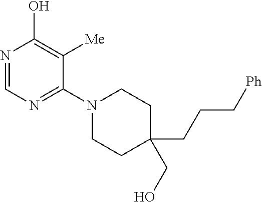 [SMILES: Cc1c(O)ncnc1N1CCC(CO)(CCCc2ccccc2)CC1]

The title compound was prepared according to General Procedure 1 from (4-(3-phenylpropyl)piperidin-4-yl)methanol hydrochloride (233 mg, 0.864 mmol) and 6-chloro-5-methylpyrimidin-4-ol (159 mg, 1.10 mmol) using K₂CO₃ (346 mg, 2.50 mmol) in DMSO (2.0 mL) at 120° C. for 23 followed by 150° C. for 16 h and was purified by trituration (EtOAc) to give a pink solid (27 mg, 9%). ¹H NMR (600 MHz, CDCl₃): δ 13.37 (br s, 1H), 7.88 (s, 1H), 7.30-7.26 (m, 2H), 7.20-7.16 (m, 3H), 3.50 (s, 2H), 3.39 (ddd, J=13.3, 7.0, 3.8 Hz, 2H), 3.30 (ddd, J=12.9, 8.3, 3.6 Hz, 2H), 2.62 (t, J=7.5 Hz, 2H), 2.00 (s, 3H), 1.64-1.53 (m, 5H), 1.49 (ddt, J=16.2, 7.0, 3.7 Hz, 4H); ¹³C NMR (201 MHz, CDCl₃): δ 166.3, 164.4, 144.4, 142.5, 128.51, 128.49, 126.0, 102.2, 67.5, 44.4, 36.8, 36.0, 34.0, 32.3, 25.2, 12.6; HRMS (ESI/LC-Q-TOF): m/z calcd for C₂₀H₂₇N₃O₂ [M+H]⁺: 342.2176; found: 342.2178.

### (1-(2-(Dimethylamino)-5,6-dimethylpyrimidin-4-yl)-4-(3-phenylpropyl)piperidin-4-yl)methanol (JDDUVA44-2AP1)

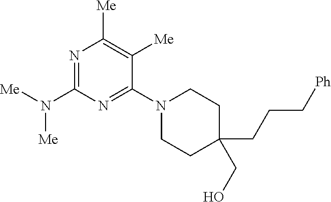 [SMILES: Cc1nc(N(C)C)nc(N2CCC(CO)(CCCc3ccccc3)CC2)c1C]

The title compound was prepared according to General Procedure 1 from (4-(3-phenylpropyl)piperidin-4-yl)methanol hydrochloride (117 mg, 0.434 mmol) and 4-chloro-N,N,5,6-tetramethylpyrimidin-2-amine (102 mg, 0.549 mmol) using K₂CO₃ (173 mg, 1.25 mmol) in DMSO (1.0 mL) at 150° C. for 18 and was purified by flash column chromatography on alumina (20% EtOAc:hexane then 15%-30% EtOAc:hexane) to give a pale yellow amorphous solid (64 mg, 39%). ¹H NMR (600 MHz, CDCl₃): δ 7.31-7.27 (m, 2H), 7.21-7.17 (m, 3H), 3.50 (s, 2H), 3.22 (ddd, J=13.0, 7.0, 3.9 Hz, 2H), 3.12 (s, 8H), 2.62 (t, J=7.6 Hz, 2H), 2.26 (s, 3H), 1.98 (s, 3H), 1.65-1.54 (m, 4H), 1.54-1.47 (m, 4H); ¹³C NMR (151 MHz; CDCl₃): δ 167.0, 165.3, 160.2, 142.6, 128.5, 128.4, 125.9, 104.1, 67.4, 44.6, 37.0, 36.8, 36.1, 34.2, 32.0, 25.2, 22.7, 14.0; HRMS (ESI/LC-Q-TOF): m/z calcd for C₂₃H₃₄N₄O [M+H]⁺: 383.2805; found: 383.2818.

### (1-(2-Aminopyrimidin-4-yl)-4-(3-phenylpropyl)piperidin-4-yl)methanol (JDDUVA49-P1)

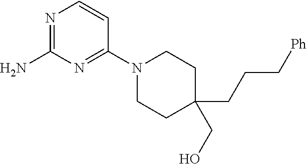 [SMILES: Nc1nccc(N2CCC(CO)(CCCc3ccccc3)CC2)n1]

The title compound was prepared according to General Procedure 1 from (4-(3-phenylpropyl)piperidin-4-yl)methanol hydrochloride (233 mg, 0.864 mmol) and 4-chloropyrimidin-2-amine (141 mg, 1.09 mmol) using K₂CO₃ (347 mg, 2.51 mmol) in DMSO (2.0 mL) at 120° C. for 19 h and was purified by flash column chromatography on alumina (2.5%-10% MeOH:EtOAc) followed by recrystallization (EtOAc) to give a yellow solid (74 mg, 26%). ¹H NMR (600 MHz, CDCl₃): δ 7.81 (d, J=6.1 Hz, 1H), 7.30-7.26 (m, 2H), 7.20-7.16 (m, 3H), 5.91 (d, J=6.2 Hz, 1H), 4.69 (s, 2H), 3.61 (dd, J=13.1, 6.7 Hz, 2H), 3.48 (s, 2H), 3.42 (ddd, J=13.0, 8.3, 4.0 Hz, 2H), 2.61 (t, J=7.6 Hz, 2H), 2.14 (br s, 1H), 1.64-1.56 (m, 2H), 1.52-1.42 (m, 6H); ¹³C NMR (201 MHz, CDCl₃): δ 162.74, 162.65, 156.5, 142.5, 128.50, 128.47, 126.0, 94.5, 67.1, 39.8, 36.7, 36.1, 33.9, 31.5, 25.2; HRMS (ESI/LC-Q-TOF): m/z calcd for C₁₉H₂₆N₄O [M+H]⁺: 327.2179; found: 327.2196.

### (1-(6-Chloro-5-methylpyrimidin-4-yl)-4-(3-phenylpropyl)piperidin-4-yl)methanol (JDDUVA50-P1)

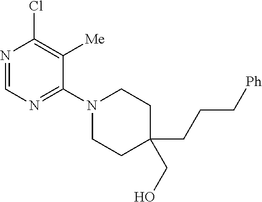 [SMILES: Cc1c(Cl)ncnc1N1CCC(CO)(CCCc2ccccc2)CC1]

The title compound was prepared according to General Procedure 1 from (4-(3-phenylpropyl)piperidin-4-yl)methanol hydrochloride (233 mg, 0.864 mmol) and 4,6-dichloro-5-methylpyrimidine (179 mg, 1.10 mmol) using K₂CO₃ (346 mg, 2.50 mmol) in DMSO (2.0 mL) at 120° C. for 23 h and was purified by flash column chromatography on alumina (30%-50% EtOAc:hexane) to give a white solid (218 mg, 70%). ¹H NMR (600 MHz, CDCl₃): δ 8.34 (s, 1H), 7.30-7.26 (m, 2H), 7.20-7.16 (m, 3H), 3.51 (s, 2H), 3.39 (ddd, J=13.3, 7.2, 3.8 Hz, 2H), 3.29 (ddd, J=13.3, 8.4, 3.6 Hz, 2H), 2.61 (t, J=7.6 Hz, 2H), 2.22 (d, J=0.7 Hz, 3H), 1.73 (br s, 1H), 1.64-1.58 (m, 4H), 1.55-1.48 (m, 4H); ¹³C NMR (151 MHz, CDCl₃): δ 166.9, 160.7, 154.5, 142.4, 128.5, 128.4, 126.0, 116.2, 67.2, 44.6, 36.7, 35.9, 34.0, 31.8, 25.2, 16.5; HRMS (ESI/LC-Q-TOF): m/z calcd for C₂₀H₂₆ClN₃O [M+H]⁺: 360.1837; found: 360.1839.

### (1-(4,6-Dimethoxy-1,3,5-triazin-2-yl)-4-(3-phenylpropyl)piperidin-4-yl)methanol (JDDUVA52-P1)

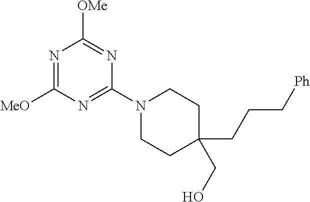 [SMILES: COc1nc(OC)nc(N2CCC(CO)(CCCc3ccccc3)CC2)n1]

The title compound was prepared according to General Procedure 1 from (4-(3-phenylpropyl)piperidin-4-yl)methanol hydrochloride (233 mg, 0.864 mmol) and 2-chloro-4,6-dimethoxy-1,3,5-triazine (194 mg, 1.10 mmol) using K₂CO₃ (346 mg, 2.50 mmol) in DMSO (2.0 mL) at 120° C. for 22 h and was purified by flash column chromatography on alumina (30%-70% EtOAc:hexane) to give a white solid (31 mg, 10%). ¹H NMR (600 MHz, CDCl₃₁H NMR (600 MHz, Chloroform-d) δ 7.28 (dd, J=8.6, 6.5 Hz, 2H), 7.20-7.16 (m, 3H), 3.93-3.87 (m, 8H), 3.64 (ddd, J=13.1, 8.3, 4.0 Hz, 2H), 3.49 (s, 2H), 2.62 (t, J=7.5 Hz, 2H), 1.63-1.57 (m, 2H), 1.52-1.35 (m, 7H); ¹³C NM R (151 MHz, CDCl₃): δ 172.4, 166.5, 142.4, 128.50, 128.46, 126.0, 67.3, 54.5, 39.6, 36.6, 36.2, 33.7, 31.6, 25.1; HRMS (ESI/LC-Q-TOF): m/z calcd for C₂₀H₂₈N₄O₃ [M+H]⁺: 373.2234; found: 373.2250.

### (1-(3-Methylpyrazin-2-yl)-4-(3-phenylpropyl)piperidin-4-yl)methanol (JDDUVA53-P1)

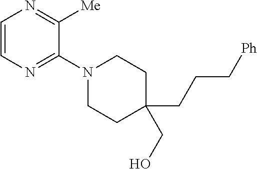 [SMILES: Cc1nccnc1N1CCC(CO)(CCCc2ccccc2)CC1]

The title compound was prepared according to General Procedure 1 from ((4-(3-phenylpropyl)piperidin-4-yl)methanol hydrochloride (233 mg, 0.864 mmol) and 2-chloro-3-methylpyrazine (0.11 mL, 1.1 mmol) using K₂CO₃ (346 mg, 2.50 mmol) in DMSO (2.0 mL) at 150° C. for 22 h and was purified by flash column chromatography on alumina (Combiflash 10%-20% EtOAc:hexane then 5%-50% EtOAc:hexane) to give a yellow oil (149 mg, 53%). ¹H NMR (600 MHz, CDCl₃): δ 8.03 (dd, J=2.6, 0.7 Hz, 1H), 8.01 (d, J=2.7 Hz, 1H), 7.30-7.26 (m, 2H), 7.20-7.16 (m, 3H), 3.53 (s, 2H), 3.17 (ddd, J=12.8, 6.9, 3.9 Hz, 2H), 3.12 (ddd, J=12.6, 8.2, 3.6 Hz, 2H), 2.62 (t, J=7.6 Hz, 2H), 2.50 (d, J=0.7 Hz, 3H), 1.67-1.60 (m, 4H), 1.59-1.51 (m, 5H); ¹³C NMR (151 MHz, CDCl₃): δ 158.4, 147.4, 142.6, 139.2, 136.4, 128.48, 128.47, 125.9, 67.5, 45.3, 36.8, 35.9, 34.0, 32.1, 25.2, 21.9; HRMS (ESI/LC-Q-TOF): m/z calcd for C₂₀H₂₇N₃O [M+H]⁺: 326.2227; found: 326.2228.

### (1-(6-Aminopyrazin-2-yl)-4-(3-phenylpropylpiperidin-4-yl)methanol (JDDUVA55-P1)

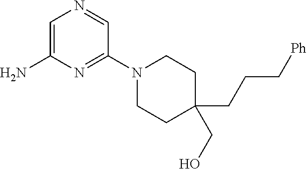 [SMILES: Nc1cncc(N2CCC(CO)(CCCc3ccccc3)CC2)n1]

The title compound was prepared according to General Procedure 1 from (4-(3-phenylpropyl)piperidin-4-yl)methanol hydrochloride (117 mg, 0.434 mmol) and 6-chloropyrazin-2-amine (71 mg, 0.55 mmol) using K₂CO₃ (173 mg, 1.25 mmol) in DMSO (1.0 mL) at 150° C. for 19 h and was purified by flash column chromatography on alumina (Combiflash 20%-100% EtOAc:hexane then 40%-50% EtOAc:hexane then 0%-100% EtOAc:hexane followed by 1%-35% MeOH:EtOAc) to give a yellow amorphous solid (54 mg, 38%). ¹H NMR (600 MHz, CDCl₃): δ 7.43 (s, 1H), 7.30-7.25 (m, 2H), 7.22 (s, 1H), 7.20-7.15 (m, 3H), 4.29 (br s, 2H), 3.56 (ddd, J=13.2, 6.9, 4.2 Hz, 2H), 3.47 (s, 2H), 3.36 (ddd, J=12.9, 8.4, 4.0 Hz, 2H), 2.61 (t, J=7.6 Hz, 2H), 2.25 (br s, 1H), 1.65-1.56 (m, 2H), 1.55-1.43 (m, 6H); ¹³C NMR (151 MHz, CDCl₃): δ 154.0, 152.8, 142.5, 128.45, 128.44, 125.9, 119.3, 118.6, 67.2, 40.4, 36.7, 35.9, 33.8, 31.3, 25.1; HRMS (ESI/LC-Q-TOF): m/z calcd for C₁₉H₂₆N₄O [M+H]⁺: 327.2179; found: 327.2195.

### (1-(5,6-Diaminopyrimidin-4-yl)-4-(3-phenylpropyl)piperidin-4-yl)methanol (JDDUVA51-P1)

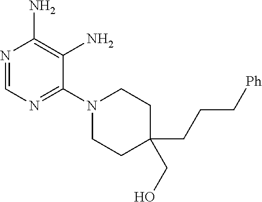 [SMILES: Nc1ncnc(N2CCC(CO)(CCCc3ccccc3)CC2)c1N]

The title compound was prepared according to General Procedure 1 from (4-(3-phenylpropyl)piperidin-4-yl)methanol hydrochloride (233 mg, 0.864 mmol) and 6-chloropyrimidine-4,5-diamine (159 mg, 1.10 mmol) using K₂CO₃ (346 mg, 2.50 mmol) in DMSO (2.0 mL) at 120° C. for 19 h followed by 150° C. for 21 h and was purified by flash column chromatography on alumina (Combiflash 0%-100% MeOH:EtOAc) followed by recrystallization (CHCl₃) to give an orange solid (75 mg, 26%). ¹H-NMR (600 MHz, CD₃OD): δ 7.76 (s, 1H), 7.23 (tt, J=7.6, 1.7 Hz, 2H), 7.20-7.17 (m, 2H), 7.14-7.10 (m, 1H), 3.46 (s, 2H), 3.07 (ddd, J=11.3, 7.1, 3.8 Hz, 2H), 3.01 (ddd, J=12.3, 8.2, 3.6 Hz, 2H), 2.60 (t, J=7.6 Hz, 2H), 1.66-1.58 (m, 4H), 1.55-1.47 (m, 4H); ¹³C NMR (151 MHz; CD₃OD): δ 154.90, 154.85, 148.0, 143.9, 129.4, 129.3, 126.7, 117.4, 67.4, 45.2, 37.8, 36.7, 35.3, 33.3, 26.1; HRMS (ESI/LC-Q-TOF): m/z calcd for C₁₉H₂₇N₅O [M+H]⁺: 342.2288; found: 342.2297.

### (1-(6-Chloropyrimidin-4-yl)-4-(3-phenylpropyl)piperidin-4-yl)methanol (JDDUVA60-P1)

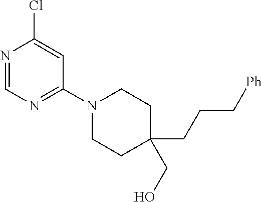 [SMILES: OCC1(CCCc2ccccc2)CCN(c2cc(Cl)ncn2)CC1]

The title compound was prepared according to General Procedure 1 from (4-(3-phenylpropyl)piperidin-4-yl)methanol hydrochloride (233 mg, 0.864 mmol) and 4,6-dichloropyrimidine (164 mg, 1.10 mmol) using K₂CO₃ (346 mg, 2.50 mmol) in DMSO (2.0 mL) at 120° C. for 17 h and was purified by recrystallization (EtOAc at −20° C.) to give a yellow solid (264 mg, 89%). ¹H-NMR (600 MHz, CDCl₃): δ 8.32 (d, J=0.9 Hz, 1H), 7.30-7.25 (m, 2H), 7.20-7.16 (m, 3H), 6.44 (d, J=1.0 Hz, 1H), 3.66 (br s, 2H), 3.49-3.45 (m, 4H), 2.61 (t, J=7.6 Hz, 2H), 2.04 (br s, 1H), 1.64-1.52 (m, 4H), 1.51-1.44 (m, 4H); ¹³C NMR (151 MHz, CDCl₃): δ 162.3, 160.0, 158.2, 142.3, 128.53, 128.46, 126.1, 101.4, 67.2, 40.3, 36.6, 36.1, 33.9, 31.3, 25.2; HRMS (ESI/LC-Q-TOF): m/z calcd for C₁₉H₂₄ClN₃O [M+H]⁺: 346.1681; found: 346.1683.

### (1-(6-Chloro-5-methoxypyrimidin-4-yl)-4-(3-phenylpropyl)piperidin-4-yl)methanol (JDDUVA67-P1)

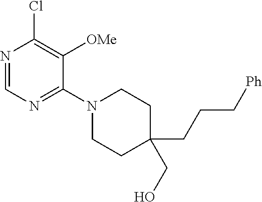 [SMILES: COc1c(Cl)ncnc1N1CCC(CO)(CCCc2ccccc2)CC1]

The title compound was prepared according to General Procedure 1 from (4-(3-phenylpropyl)piperidin-4-yl)methanol hydrochloride (117 mg, 0.434 mmol) and 4,6-dichloro-5-methoxypyrimidine (99 mg, 0.55 mmol) using K₂CO₃ (173 mg, 1.25 mmol) in DMSO (1.0 mL) at 120° C. for 21 h and was purified by column chromatography on alumina (Combiflash 0%-20% EtOAc:hexane) to give a colorless oil (83 mg, 51%). ¹H NMR (600 MHz, CDCl₃): δ 8.13 (s, 1H), 7.30-7.26 (m, 2H), 7.20-7.16 (m, 3H), 3.86 (ddd, J=13.5, 7.2, 3.8 Hz, 2H), 3.71 (s, 3H), 3.65 (ddd, J=13.5, 8.5, 3.6 Hz, 2H), 3.50 (s, 2H), 2.62 (t, J=7.6 Hz, 2H), 1.70 (br s, 1H), 1.63-1.54 (m, 4H), 1.52-1.46 (m, 4H); ¹³C NMR (151 MHz, CDCl₃): δ 157.4, 152.8, 152.2, 142.4, 137.0, 128.48, 128.45, 126.0, 67.2, 59.6, 42.4, 36.7, 36.0, 34.0, 32.1, 25.1; HRMS (ESI/LC-Q-TOF): m/z calcd for C₂₀H₂₆ClN₃O₂[M+H]⁺: 376.1786; found: 376.1790.

### (1-(6-Ethyl-5-fluoropyrimidin-4-yl)-4-(3-phenylpropyl)piperidin-4-yl)methanol (JDDUVA68-P1)

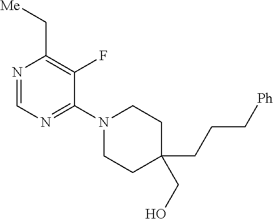 [SMILES: CCc1ncnc(N2CCC(CO)(CCCc3ccccc3)CC2)c1F]

The title compound was prepared according to General Procedure 1 from (4-(3-phenylpropyl)piperidin-4-yl)methanol hydrochloride (117 mg, 0.434 mmol) and 4-chloro-6-ethyl-5-fluoropyrimidine (69 L, 0.55 mmol) using K₂CO₃ (173 mg, 1.25 mmol) in DMSO (1.0 mL) at 120° C. for 18 h and was purified by column chromatography on alumina (Combiflash; 0%-20% EtOAc:hexane and then 0%-15% EtOAc:hexane) to give a pale yellow oil (114 mg, 74%). ¹H NMR (600 MHz, CDCl₃): δ 8.27 (d, J=2.5 Hz, 1H), 7.30-7.26 (m, 2H), 7.20-7.16 (m, 3H), 3.77 (ddd, J=13.6, 7.3, 3.9 Hz, 2H), 3.59 (ddd, J=13.6, 8.4, 3.7 Hz, 2H), 3.50 (d, J=4.4 Hz, 2H), 2.70 (qd, J=7.6, 2.8 Hz, 2H), 2.62 (t, J=7.5 Hz, 2H), 1.79 (t, J=5.5 Hz, 1H), 1.64-1.54 (m, 4H), 1.52-1.45 (m, 4H), 1.25 (t, J=7.6 Hz, 3H); ¹³C NMR (151 MHz, CDCl₃): δ 156.1 (d, J=15.8 Hz), 152.7 (d, J=9.3 Hz), 151.7 (d, J=5.1 Hz), 144.6 (d, J=257.1 Hz), 142.4, 128.49, 128.46, 126.0, 67.2, 42.3 (d, J=7.8 Hz), 36.7, 36.1, 34.0, 31.9, 25.2, 24.1, 12.4 (d, J=1.0 Hz); 19F NMR (564 MHz, CDCl3): δ −146.0 (q, J=2.7 Hz); HRMS (ESI/LC-Q-TOF): m/z calcd for C₂₁H₂₈FN₃O [M+H]⁺: 358.2289; found: 358.2292.

### (1-(4-Aminopyrimidin-2-yl)-4-(3-phenylpropyl)piperidin-4-yl)methanol (JDDUVA69-P1)

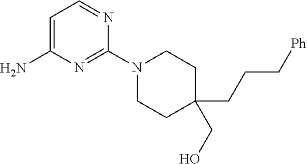 [SMILES: Nc1ccnc(N2CCC(CO)(CCCc3ccccc3)CC2)n1]

The title compound was prepared according to General Procedure 1 from (4-(3-phenylpropyl)piperidin-4-yl)methanol hydrochloride (117 mg, 0.434 mmol) and 2-chloropyrimidin-4-amine (71 mg, 0.55 mmol) using K₂CO₃ (173 mg, 1.25 mmol) in DMSO (1.0 mL) at 120° C. for 18 h and was purified by column chromatography on alumina (Combiflash 30%-100% EtOAc:hexane) to give a pale yellow solid (67 mg, 48%). ¹H NMR (600 MHz, CDCl₃): δ 7.90 (d, J=5.6 Hz, 1H), 7.29-7.26 (m, 2H), 7.19-7.16 (m, 3H), 5.70 (d, J=5.6 Hz, 1H), 4.60 (s, 2H), 3.81 (ddd, J=13.5, 6.9, 4.2 Hz, 2H), 3.57 (ddd, J=13.5, 8.2, 4.1 Hz, 2H), 3.47 (s, 2H), 2.60 (t, J=7.6 Hz, 2H), 1.79 (br s, 1H), 1.64-1.55 (m, 2H), 1.45 (dddd, J=25.9, 13.5, 7.0, 3.6 Hz, 6H); ¹³C NMR (201 MHz, CDCl₃): δ 163.4, 161.9, 156.9, 142.6, 128.5, 128.4, 125.9, 94.4, 67.4, 39.6, 36.7, 36.1, 33.8, 31.7, 25.1; HRMS (ESI/LC-Q-TOF): m/z calcd for C₁₉H₂₆N₄O [M+H]⁺: 327.2179; found: 327.2183.

### (1-(4,6-Diamino-1,3,5-triazin-2-yl)-4-(3-phenylpropyl)piperidin-4-yl)methanol (JDDUVA78-P1)

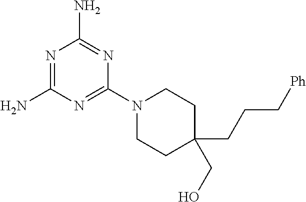 [SMILES: Nc1nc(N)nc(N2CCC(CO)(CCCc3ccccc3)CC2)n1]

The title compound was prepared according to General Procedure 1 from (4-(3-phenylpropyl)piperidin-4-yl)methanol hydrochloride (117 mg, 0.434 mmol) and 6-chloro-1,3,5-triazine-2,4-diamine (80 mg, 0.55 mmol) using K₂CO₃ (173 mg, 1.25 mmol) in DMSO (1.0 mL) at 120° C. for 19 h and was purified by recrystallization (DCM at −20° C.) to give a pale yellow solid (66 mg, 45%). ¹H NMR (600 MHz, MeOD): ¹H NMR (600 MHz, CD₃OD) δ 7.25-7.22 (m, 2H), 7.19-7.16 (m, 2H), 7.14-7.11 (m, 1H), 3.79 (ddd, J=13.5, 7.1, 4.0 Hz, 2H), 3.54-3.47 (m, 2H), 3.41 (s, 2H), 2.59 (t, J=7.6 Hz, 2H), 1.62-1.54 (m, 2H), 1.48-1.39 (m, 4H), 1.35 (ddd, J=13.6, 7.1, 3.9 Hz, 2H); ¹³C NMR (201 MHz, CD₃OD): δ 168.5, 166.3, 143.8, 129.4, 129.3, 126.7, 67.3, 40.1, 37.7, 37.1, 35.0, 32.8, 26.1; HRMS (ESI/LC-Q-TOF): m/z calcd for C₁₈H₂₆N₆O [M+H]⁺: 343.2241; found: 343.2247.

### 4-(Piperidin-1-yl)pyrimidin-2-amine (JDDUVA12-P1)

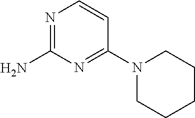 [SMILES: Nc1nccc(N2CCCCC2)n1]

The title compound was prepared according to General Procedure 1 from piperidine (1.63 mL, 16.5 mmol) and 4-chloropyrimidin-2-amine (1.93 g, 15.0 mmol) using K₂CO₃ (3.11 g, 22.5 mmol) in NMP (6.0 mL) at 120° C. for 18 h and purified by flash column chromatography on silica (1% NEt₃:DCM) followed by recrystallization (EtOAc:hexane) to give an pale orange solid (1.01 g, 38%). ¹H-NMR (600 MHz, CDCl₃): δ 7.80 (d, J=6.2 Hz, 1H), 5.90 (d, J=6.2 Hz, 1H), 4.80 (s, 2H), 3.57-3.46 (m, 4H), 1.67-1.60 (m, 2H), 1.57-1.50 (m, 4H); ¹³C NMR (151 MHz, CDCl₃): δ 163.0, 162.6, 156.6, 94.3, 44.9, 25.6, 24.8; HRMS (ESI/LC-Q-TOF): m/z calcd for C₉H₁₄N₄[M+H]⁺: 179.1291; found: 179.1299.

### 4-(4-Methylpiperidin-1-yl)pyrimidin-2-amine (JDDUVA59-P1)

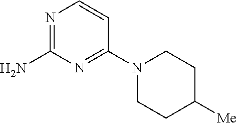 [SMILES: CC1CCN(c2ccnc(N)n2)CC1]

The title compound was prepared according to General Procedure 1 from 4-methylpiperidine (0.12 mL, 1.0 mmol) and 4-chloropyrimidin-2-amine (141 mg, 1.09 mmol) using K₂CO₃ (346 mg, 2.50 mmol) in DMSO (2.0 mL) at 120° C. for 19 h and was purified by recrystallization (EtOAc at −20° C.) to give a pale yellow solid (131 mg, 68%). ¹H-NMR (600 MHz, CDCl₃): δ 7.81 (d, J=6.2 Hz, 1H), 5.92 (d, J=6.2 Hz, 1H), 4.78 (s, 2H), 4.29 (d, J=11.8 Hz, 2H), 2.77 (ddd, J=13.3, 12.3, 2.7 Hz, 2H), 1.69-1.64 (m, 2H), 1.64-1.56 (m, 1H), 1.11 (dddd, J=13.3, 12.2, 11.1, 4.3 Hz, 2H), 0.93 (d, J=6.6 Hz, 3H); ¹³C NMR (151 MHz, CDCl₃): δ 162.8, 162.6, 156.5, 94.4, 44.3, 33.9, 31.3, 21.9; HRMS (ESI/LC-Q-TOF): m/z calcd for C₁₀H₁₆N₄ [M+H]⁺: 193.1448; found: 193.1452.

### 4-(4-Phenylpiperidin-1-yl)pyrimidin-2-amine (JDDUVA63-P1)

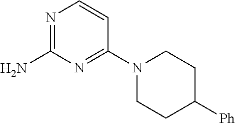 [SMILES: Nc1nccc(N2CCC(c3ccccc3)CC2)n1]

The title compound was prepared according to General Procedure 1 from 4-phenylpiperidine (161 mg, 0.998 mmol) and 4-chloropyrimidin-2-amine (141 mg, 1.09 mmol) using K₂CO₃ (346 mg, 2.50 mmol) in DMSO (2.0 mL) at 120° C. for 18 h and was purified by recrystallization (EtOAc) to give a pale yellow solid (138 mg, 54%). ¹H-NMR (600 MHz, CDCl₃): δ 7.88 (d, J=6.1 Hz, 1H), 7.31 (tq, J=7.8, 1.0 Hz, 2H), 7.24-7.19 (m, 3H), 6.00 (d, J=6.2 Hz, 1H), 4.74 (s, 2H), 4.51 (d, J=12.0 Hz, 2H), 2.91 (td, J=13.0, 2.6 Hz, 2H), 2.78 (tt, J=12.2, 3.7 Hz, 1H), 1.94-1.89 (m, 2H), 1.68 (dtd, J=13.4, 12.4, 4.3 Hz, 2H); ¹³C NMR (151 MHz, CDCl₃): δ 163.0, 162.7, 156.9, 145.6, 128.7, 126.9, 126.6, 94.4, 44.7, 43.1, 33.1; HRMS (ESI/LC-Q-TOF): m/z calcd for C₁₅H₁₈N₄[M+H]⁺: 255.1604; found: 255.1616.

### (1-(2-Aminopyrimidin-4-yl)piperidin-2-yl)methanol (JDDUVA61-P2)

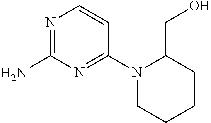 [SMILES: Nc1nccc(N2CCCCC2CO)n1]

The title compound was prepared according to General Procedure 1 from 2-piperidinemethanol (115 mg, 0.998 mmol) and 4-chloropyrimidin-2-amine (141 mg, 1.09 mmol) using K₂CO₃ (346 mg, 2.50 mmol) in DMSO (2.0 mL) at 120° C. for 17 h and was purified by recrystallization (EtOAc) after the aqueous layer was back extracted (using EtOAc and brine) to give a pale yellow solid (63 mg, 30%). ¹H-NMR (600 MHz, CDCl₃): δ 7.79 (d, J=6.2 Hz, 1H), 5.98 (d, J=6.2 Hz, 1H), 4.80 (s, 2H), 4.72 (s, 1H), 4.02 (d, J=13.7 Hz, 1H), 3.91 (dd, J=10.9, 8.9 Hz, 1H), 3.69 (dd, J=11.0, 5.6 Hz, 1H), 3.63 (br s, 1H), 3.02 (ddd, J=13.5, 12.1, 3.3 Hz, 1H), 1.80-1.68 (m, 2H), 1.68-1.46 (m, 4H); ¹³C NMR (151 MHz, CDCl₃): δ 163.8, 162.3, 156.3, 95.0, 62.2, 52.7, 40.2, 25.5, 25.0, 19.6; HRMS (ESI/LC-Q-TOF): m/z calcd for C₁₀H₁₆N₄O [M+H]⁺: 209.1397; found: 209.1401.

### 4-(3,4-Dihydroisoquinolin-2(1H)-yl)pyrimidin-2-amine (JDDUVA71-P1)

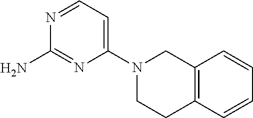 [SMILES: Nc1nccc(N2CCc3ccccc3C2)n1]

The title compound was prepared according to General Procedure 1 from 1,2,3,4-tetrahydroisoquinoline (0.14 mL, 1.0 mmol) and 2-chloropyrimidin-4-amine (141 mg, 1.09 mmol) using K₂CO₃ (346 mg, 2.50 mmol) in DMSO (2.0 mL) at 120° C. for 21 h and was purified by recrystallization (EtOAc) to give a white solid (126 mg, 56%). ¹H NMR (600 MHz, CDCl₃): δ 7.90 (d, J=6.1 Hz, 1H), 7.23-7.15 (m, 4H), 5.99 (d, J=6.1 Hz, 1H), 4.78 (s, 2H), 4.70 (s, 2H), 3.80 (t, J=5.9 Hz, 2H), 2.92 (t, J=5.9 Hz, 2H); ¹³C NMR (151 MHz, CDCl₃): δ 162.7, 162.5, 156.7, 135.3, 133.8, 128.5, 126.8, 126.6, 126.5, 94.6, 46.0, 41.7, 29.0; HRMS (ESI/LC-Q-TOF): m/z calcd for C₁₃H₁₄N₄[M+H]⁺: 227.1291; found: 227.1302.

### 4-(4-(3-Phenylpropyl)piperidin-1-yl)pyrimidin-2-amine (MLKUVA13)

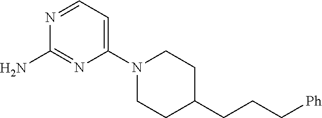 [SMILES: Nc1nccc(N2CCC(CCCc3ccccc3)CC2)n1]

The title compound was prepared according to General Procedure 1 from 4-(3-phenylpropyl)piperidine (0.06 mL, 0.28 mmol) and 4-chloropyrimidin-2-amine (36 mg, 0.28 mmol) using K₂CO₃ (87 mg, 0.63 mmol) in DMSO (1.0 mL) at 80° C. for 20 h and was purified by recrystallization (EtOAc at −20° C.) to give a pale yellow solid (37 mg, 45%). ¹H NMR (600 MHz, CDCl₃): δ 7.83 (d, J=6.2 Hz, 1H), 7.30-7.26 (m, 2H), 7.20-7.15 (m, 3H), 5.93 (d, J=6.2 Hz, 1H), 4.69 (s, 2H), 4.31 (d, J=12.9 Hz, 2H), 2.77 (td, J=12.9, 2.7 Hz, 2H), 2.60 (t, J=7.7 Hz, 2H), 1.73 (d, J=13.0 Hz, 2H), 1.68-1.61 (m, 2H), 1.52 (ttt, J=10.7, 7.0, 3.8 Hz, 1H), 1.33-1.27 (m, 2H), 1.12 (qd, J=12.8, 4.3 Hz, 2H); ¹³C NMR (151 MHz, CDCl₃): δ 162.8, 162.6, 156.6, 142.6, 128.5, 128.4, 125.8, 94.5, 44.3, 36.3, 36.2, 32.0, 28.7; HRMS (ESI/LC-Q-TOF): m/z calcd for C₁₈H₂₄N₄[M+H]⁺: 297.2074; found 297.2075.

### Example 7. General Procedure 2: Suzuki Cross-Coupling of Derivatives

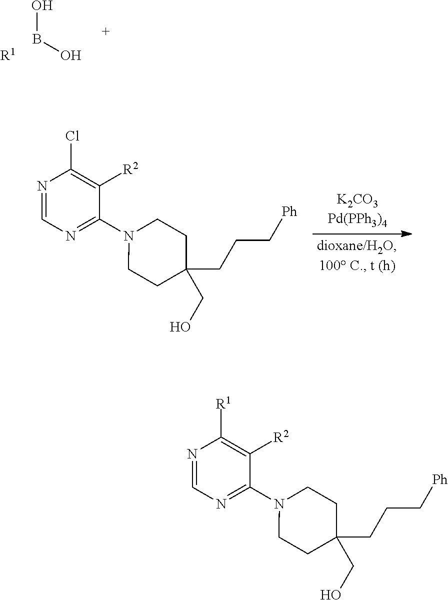

Method A: Aryl chloride, boronic acid, K₂CO₃, dioxane, and water combined in a pressure flask. The solution was degassed with a stream of nitrogen for 15 min. Palladium was added and the vessel was flushed with nitrogen, sealed, and heated to 100° C. for the indicated time. Following that time, the reaction mixture was cooled, diluted with H₂O, and extracted with EtOAc. The organic layers were combined and washed with brine, dried with a drying agent, filtered, and concentrated. Purification of the residue by the noted method(s) gave the corresponding product.

Method B: An aryl chloride, boronic acid (2 equiv), Pd(PPh₃)₄ (5 mol %), and K₂CO₃ (3 equiv) were combined in a vial. The vial was sealed and purged with N₂. Dioxane and degassed H₂O were added (1:1 ratio), and the vial was placed in a preheated oil bath to heat with stirring at 100° C. for the indicated time. Following that time, the reaction mixture was cooled, diluted with H₂O, and extracted with DCM. The organic layers were combined and washed with brine, dried with a drying agent, filtered, and concentrated. Purification of the residue by the noted method(s) gave the corresponding product.

### (1-(5,6-Dimethylpyrimidin-4-yl)-3-(3-phenylpropyl)piperidin-3-yl)methanol (PLH2056)

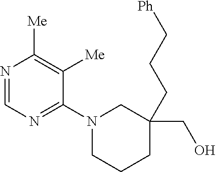 [SMILES: Cc1ncnc(N2CCCC(CO)(CCCc3ccccc3)C2)c1C]

The title compound was prepared according to General Procedure 2 Method A from (1-(6-chloro-5-methylpyrimidin-4-yl)-3-(3-phenylpropyl)piperidin-3-yl)methanol (357.9 mg, 0.99 mmol), methylboronic acid (120.5 mg, 2.01 mmol), dioxane (0.75 mL), water (0.75 mL), K₂CO₃ (414.9 mg, 3.00 mmol), and Tetrakis(triphenylphosphine)palladium(0) (57.9 mg, 0.050 mmol). Reaction ran at 100° C. for 16.3 h. Purified by flash chromatography on silica (33% Acetone/Hexane) to give a clear oil that slowly crystalized to a white solid (88.2 mg, 26%). ¹H NMR (600 MHz, CDCl₃): δ 8.36 (s, 1H), 7.28-7.24 (m, 2H), 7.19-7.14 (m, 3H), 4.85 (s, 1H), 3.98 (dt, J=13.7, 1.9 Hz, 1H), 3.58 (ddd, J=11.6, 8.1, 3.4 Hz, 2H), 3.28 (t, J=10.5 Hz, 1H), 3.12 (ddd, J=12.9, 10.8, 4.0 Hz, 1H), 2.70 (d, J=13.7 Hz, 1H), 2.66-2.53 (m, 2H), 2.35 (s, 3H), 2.08 (s, 3H), 1.72-1.55 (m, 2H), 1.51 (dddd, J=18.5, 10.8, 6.2, 3.0 Hz, 3H), 1.39-1.32 (m, 1H), 1.25-1.16 (m, 1H); ¹³C NMR (151 MHz, CDCl₃): δ 165.44, 164.47, 154.11, 142.71, 128.51, 128.39, 125.81, 114.57, 62.82, 53.70, 49.69, 38.05, 36.88, 35.91, 30.80, 30.45, 25.08, 22.78, 15.83; HRMS (ESI/LC-Q-TOF): m/z calcd for C₂₁H₂₉N₃O [M+H]⁺:340.2390; found 340.2393.

### (1-(5-methyl-6-phenylpyrimidin-4-yl)-3-(3-phenylpropyl)piperidin-3-yl)methanol (PLH2063)

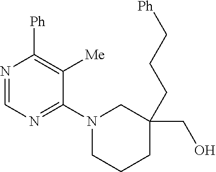 [SMILES: Cc1c(-c2ccccc2)ncnc1N1CCCC(CO)(CCCc2ccccc2)C1]

The title compound was prepared according to General Procedure 2 Method A from (1-(6-chloro-5-methylpyrimidin-4-yl)-3-(3-phenylpropyl)piperidin-3-yl)methanol (182.4 mg, 0.51 mmol), phenylboronic acid (122.9 mg, 1.01 mmol), dioxane (0.5 mL), water (0.5 mL), K₂CO₃ (209.7 mg, 1.52 mmol), and Tetrakis(triphenylphosphine)palladium(0) (30.0 mg, 0.026 mmol). Reaction ran at 100° C. for 5.5 h. Purified by flash chromatography on silica (33% to 50% to 75% EtOAc/Hexane) to give a foamy solid (163.8 mg, 82%). ¹H NMR (600 MHz, CDCl₃): δ 8.53 (d, J=1.7 Hz, 1H), 7.55 (dt, J=7.8, 1.9 Hz, 2H), 7.46-7.37 (m, 3H), 7.29-7.22 (m, 2H), 7.21-7.12 (m, 3H), 4.98 (s, 1H), 4.04 (d, J=13.6 Hz, 1H), 3.72 (d, J=12.4 Hz, 1H), 3.60 (d, J=11.7 Hz, 1H), 3.30 (dd, J=12.3, 5.0 Hz, 1H), 3.19 (tt, J=12.7, 2.6 Hz, 1H), 2.78 (d, J=13.6 Hz, 1H), 2.69-2.51 (m, 2H), 2.12 (d, J=1.8 Hz, 3H), 1.75-1.49 (m, 6H), 1.38 (dtd, J=14.3, 6.6, 3.7 Hz, 1H), 1.29-1.18 (m, 1H); ¹³C NMR (151 MHz, CDCl₃): δ 166.19, 165.49, 154.48, 142.53, 139.08, 129.36, 129.03, 128.38, 128.30, 128.27, 128.20, 125.69, 114.09, 62.70, 53.77, 49.35, 38.10, 36.74, 35.59, 30.64, 24.94, 22.63, 18.46; HRMS (ESI/LC-Q-TOF): m/z calcd for C₂₆H₃₁N₃O [M+H]⁺: 402.2547; found 402.2558.

### (1-(5,6-Dimethylpyrimidin-4-yl)-4-(3-phenylpropyl)piperidin-4-yl)methanol (JDDUVA96-P1)

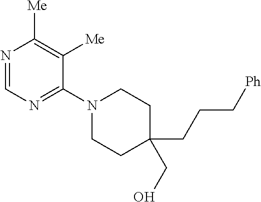 [SMILES: Cc1ncnc(N2CCC(CO)(CCCc3ccccc3)CC2)c1C]

The title compound was prepared according to General Procedure 2 Method B from (1-(6-chloro-5-methylpyrimidin-4-yl)-4-(3-phenylpropyl)piperidin-4-yl)methanol (180 mg, 0.500 mmol) and methylboronic acid (60 mg, 1.0 mmol) at 100° C. for 21 h and was purified by column chromatography on alumina (30%-100% EtOAc:hexane to 10% MeOH:EtOAc) followed by recrystallization (EtOAc at −20° C.) to give a pale yellow solid (46 mg, 27%). ¹H NMR (600 MHz; CDCl₃): δ 8.50 (s, 1H), 7.30-7.26 (m, 2H), 7.18 (ddt, J=7.1, 3.3, 1.6 Hz, 3H), 3.51 (s, 2H), 3.28 (ddd, J=13.2, 7.1, 3.9 Hz, 2H), 3.20 (ddd, J=12.8, 8.4, 3.6 Hz, 2H), 2.61 (t, J=7.6 Hz, 2H), 2.39 (s, 3H), 2.12 (s, 3H), 1.69 (br s, 1H), 1.60 (ddt, J=10.8, 6.6, 3.1 Hz, 4H), 1.52 (ddt, J=12.2, 8.7, 4.3 Hz, 4H); ¹³C NMR (151 MHz, CDCl₃): δ 166.4, 164.7, 154.6, 142.5, 128.49, 128.47, 126.0, 117.0, 67.4, 44.8, 36.8, 36.0, 34.0, 32.0, 25.2, 22.5, 15.0; HRMS (ESI/LC-Q-TOF): m/z calcd for C₂₁H₂₉N₃O [M+H]⁺: 340.2383; found: 340.2401.

### Synthesis of (1-(2-Aminopyrimidin-4-yl)piperidin-3-yl)methanol (JDDUVA18-P1)

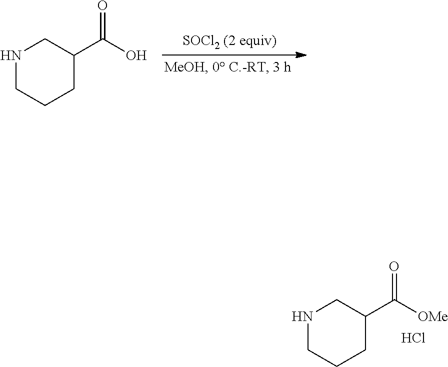 [SMILES: O=C(O)C1CCCNC1 ; COC(=O)C1CCCNC1 ; Cl]

Piperidine-3-carboxylic acid (2.71 g, 21.0 mmol) and MeOH (100 mL) were added to a flask and the flask was purged. The mixture was cooled to 0° C. and thionyl chloride (3.1 mL, 42 mmol) was slowly added. Once added, the mixture was allowed to warm to RT with stirring for 3 h. The mixture was concentrated in vacuo to give a white solid (3.69 g, 98%) that was used without further purification.

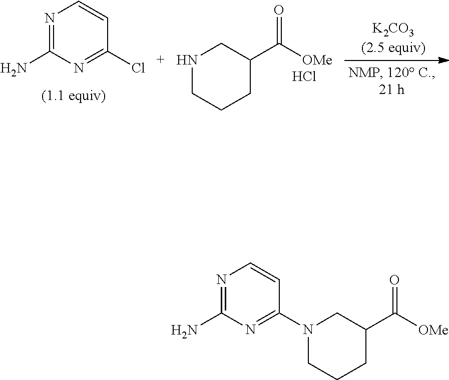 [SMILES: Nc1nccc(Cl)n1 ; COC(=O)C1CCCNC1 ; Cl ; COC(=O)C1CCCN(c2ccnc(N)n2)C1]

4-Chloropyrimidin-2-amine (1.41 g, 10.9 mmol), methyl piperidine-3-carboxylate hydrochloride (1.80 g, 10.0 mmol), and K₂CO₃ (3.46 g, 25.0 mmol), and NMP (8.0 mL) were combined in a pressure vial and the vial was capped. The mixture was allowed to stir at 120° C. for 21 h before being cooled and diluted with DCM. The residual solids were filtered and rinsed with DCM and the filtrate was washed with H₂O (40 mL) before being dried with MgSO₄, filtered, and concentrated. The residue was purified by flash column chromatography on silica (49% acetone:2% NEt₃:49% hexane to 2% NEt₃:acetone) to give an orange solid (517 mg, 22%) that was used in the next step without further purification.

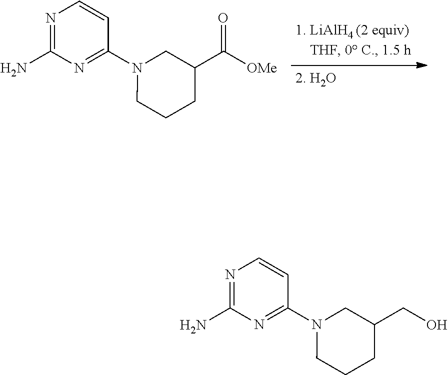 [SMILES: COC(=O)C1CCCN(c2ccnc(N)n2)C1 ; Nc1nccc(N2CCCC(CO)C2)n1]

LiAlH₄ (0.80 mL, 2.4 M soln in THF, 1.9 mmol) was added to THF (5 mL) in a purged flask and the solution was cooled to 0° C. Methyl 1-(2-aminopyrimidin-4-yl)piperidine-3-carboxylate (236 mg, 0.999 mmol) in THF (3 mL) was slowly added and the mixture was stirred at 0° C. for 1.5 h. The reaction was quenched with the sequential addition of H₂O (1 mL), NaOH (1 M, 2 mL), and H₂O (3 mL) and the mixture was allowed to stir for about 20 min before being filtered through a plug of celite with DCM and concentrated in vacuo. The residue was recrystallized (EtOAc/hexane) to give the product as a white solid (153 mg, 74%). ¹H NMR (600 MHz; CD₃OD): δ 7.71-7.67 (m, 1H), 6.07 (d, J=6.4 Hz, 1H), 4.26 (dd, J=72.8, 9.3 Hz, 2H), 3.48 (dd, J=10.9, 5.5 Hz, 1H), 3.41 (dd, J=10.9, 7.7 Hz, 1H), 2.96 (ddd, J=13.6, 11.4, 2.7 Hz, 1H), 2.76 (dd, J=13.1, 10.2 Hz, 1H), 1.83 (dt, J=13.0, 4.2 Hz, 1H), 1.75-1.64 (m, 2H), 1.52-1.44 (m, 1H), 1.30 (qd, J=11.2, 3.9 Hz, 1H); ¹³C NMR (151 MHz, CD₃OD): δ 164.0, 163.9, 156.1, 94.8, 65.5, 39.8, 28.5, 25.5; HRMS (ESI/LC-Q-TOF): m/z calcd for C₂₀H₂₆N₄O [M+H]⁺: 209.1397; found: 209.1399.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A compound having a structure represented by a formula: 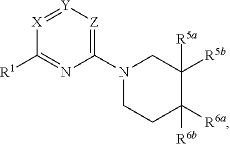
wherein X is selected from N and CR³⁰; and
wherein R³⁰ is selected from hydrogen, halogen, amino, hydroxy, cyano, nitro, —SF₅, aryl, C1-C3 alkyl, C1-C3 alkoxy, C1-C3 aminoalkyl, C1-C3 hydroxyalkyl, and C1-C3 haloalkyl;
wherein Y is selected from N and CR⁴⁰;
wherein R⁴⁰ is selected from hydrogen, halogen, amino, hydroxy, cyano, nitro, —SF₅, aryl, C1-C3 alkyl, C1-C3 alkoxy, C1-C3 aminoalkyl, C1-C3 hydroxyalkyl, and C1-C3 haloalkyl; or
wherein R³⁰ and R⁴⁰ are covalently bonded and, together with the intermediate carbons, comprise an optionally substituted fused ring selected from 5- to 7-membered heteroaryl and 6-membered aryl;
wherein Z is selected from N and CR⁵⁰;
wherein R⁵⁰ is selected from hydrogen, halogen, amino, hydroxy, cyano, nitro, —SF⁵, aryl, C1-C3 alkyl, C1-C3 alkoxy, C1-C3 aminoalkyl, C1-C3 hydroxyalkyl, and C1-C3 haloalkyl; or
wherein R⁴⁰ and R⁵⁰ are covalently bonded and, together with the intermediate carbons, comprise an optionally substituted fused ring selected from 5- to 7-membered heteroaryl and 6-membered aryl;
wherein R¹ is selected from hydrogen, halogen, amino, hydroxy, cyano, nitro, —SF⁵, C1-C3 alkyl, C1-C3 alkoxy, C1-C3 aminoalkyl, C1-C3 hydroxyalkyl, C1-C3 haloalkyl;
wherein each of R^{5a} and R^{6a} is independently selected from hydrogen, C1-C6 hydoxyalkyl, C1-C6 aminoalkyl, and C1-C6 haloalkyl; and
wherein each of R^{5b} and R^{6b} is independently selected from hydrogen, —(C1-C8 alkanediyl)-aryl, —(C1-C8 alkanediyl)-heteroaryl, —(C1-C4 alkanediyl)-O—(C1-C4 alkanediyl)-aryl, and —(C1-C4 alkanediyl)-O—(C1-C4 alkanediyl)-heteroaryl provided that at least one one of R^{5b} and R^{6b} is not hydrogen;
wherein the aryl is optionally substituted with 1, 2, or 3 groups selected from halogen, C1-C3 alkyl, and C1-C3 haloalkyl; and
wherein the heteroaryl is optionally substituted with 1, 2, or 3 groups selected from halogen, C1-C3 alkyl, and C1-C3 haloalkyl;
or a pharmaceutically acceptable salt thereof.

2. The compound of claim 1, wherein X is N.

3. The compound of claim 2, wherein Y is CR⁴⁰; and wherein Z is CR⁵⁰.

4. The compound of claim 2, wherein each of Y and Z is CH.

5. The compound of claim 2, wherein Y is CR⁴⁰; and wherein Z is CH.

6. The compound of claim 2, wherein Y is CH; and wherein Z is CR⁵⁰.

7. The compound of claim 1, wherein X is N; and wherein Y is N.

8. The compound of claim 7, wherein Z is CR⁵⁰.

9. The compound of claim 7, wherein Z is CH.

10. The compound of claim 1, wherein X is N; and wherein Z is N.

11. The compound of claim 7, wherein Y is CR⁵⁰.

12. The compound of claim 7, wherein Y is CH.

13. The compound of claim 1, wherein R¹ is selected from fluoro, chloro, amino, hydroxy, cyano, nitro, —SF⁵, methyl, —NHCH₃, —N(CH₃)₂, —OCH₃, —CH₂OH, —CH₂F, and —CH₂Cl.

14. The compound of claim 1, wherein each of R^{6a} and R^{6b} are hydrogen; wherein R^{5a} is selected from hydrogen, C1-C3 hydoxyalkyl, C1-C3 aminoalkyl, and C1-C3 haloalkyl; and wherein R^{5b} is selected from —(C1-C6 alkanediyl)-aryl, —(C1-C6 alkanediyl)-heteroaryl, —(C1-C3 alkanediyl)-O—(C1-C3 alkanediyl)-aryl, and —(C1-C3 alkanediyl)-O—(C1-C3 alkanediyl)-heteroaryl.

15. The compound of claim 14, wherein the aryl is unsubstituted; or wherein the heteroaryl is unsubstituted.

16. The compound of claim 15, wherein the aryl is phenyl; or wherein the heteroaryl is pydrinyl, pyrimidinyl, pyrazinyl, or triazinyl.

17. The compound of claim 14, wherein R^{5a} is selected from methyl, —CH₂F, —CH₂Cl, —CHF₂, —CF₃, —CHCl₂, —CCl₃, —CH₂OH, —CH(OH)CH₃, —CH₂NH₂, —CH₂NHCH₃, and —CH₂N(CH₃).

18. The compound of claim 17, wherein R^{5a} is selected from —CH₂OH and —CH(OH)CH₃.

19. The compound of claim 14, wherein R^{5b} is selected from —(C3-C6 alkanediyl)-aryl, —(C3-C6 alkanediyl)-heteroaryl, —(C3 alkanediyl)-O—(C3 alkanediyl)-aryl, and —(C1-C3 alkanediyl)-O—(C1-C3 alkanediyl)-heteroaryl.

20. The compound of claim 19, wherein R^{5b} is selected from —(C3-C6 alkanediyl)-phenyl, and —(C3 alkanediyl)-O—(C3 alkanediyl)-phenyl.

21. The compound of any one of claims 1-20, wherein R³⁰ is selected from hydrogen, halogen, amino, C1-C3 alkyl, and C1-C3 haloalkyl.

22. The compound of claim 21, wherein R³⁰ is selected from hydrogen, methyl, ethyl and C1-C2 haloalkyl.

23. The compound of claim 21, wherein R³⁰ is hydrogen.

24. The compound of claim 1, wherein each of R⁴⁰ and R⁵⁰ is independently selected from halogen, amino, C1-C3 alkyl, and C1-C3 haloalkyl.

25. The compound of claim 24, wherein each of R⁴⁰ and R⁵⁰ is independently selected from methyl, ethyl and C1-C2 haloalkyl.

26. The compound of claim 24, wherein each of R⁴⁰ and R⁵⁰ is methyl.

27. The compound of claim 1, having a structure represented by a formula:

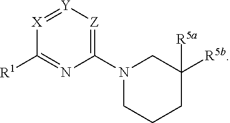

28. The compound of claim 27, wherein the compound is not present as:

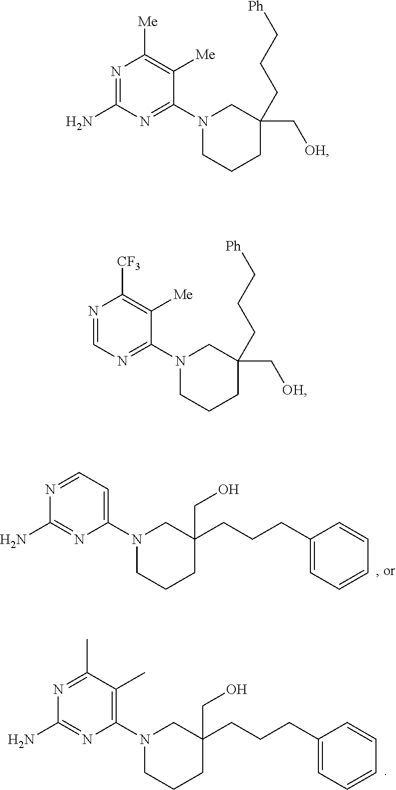 [SMILES: Cc1nc(N)nc(N2CCCC(CO)(CCCc3ccccc3)C2)c1C ; Cc1c(N2CCCC(CO)(CCCc3ccccc3)C2)ncnc1C(F)(F)F ; Nc1nccc(N2CCCC(CO)(CCCc3ccccc3)C2)n1 ; Cc1nc(N)nc(N2CCCC(CO)(CCCc3ccccc3)C2)c1C]

29. The compound of claim 27, having a structure represented by a formula:

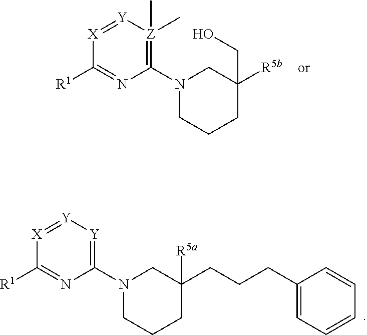

30. The compound of claim 27, having a structure represented by a formula:

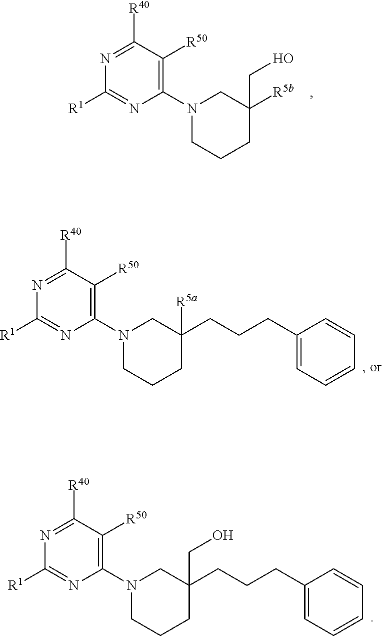

31. A pharmaceutical composition comprising a therapeutically effective amount of a compound of claim 1, or a pharmaceutically acceptable salt, solvate, or polymorph thereof, and a pharmaceutically acceptable carrier.

32. The pharmaceutical composition of claim 31, further comprising at least one agent known to treat a cancer.

33. The pharmaceutical composition of claim 32, wherein the at least one agent known to treat a cancer is a hormone therapy agent; an alkylating agent, an antimetabolite agent, an antineoplastic antibiotic agent, a mitotic inhibitor agent, a mTor inhibitor agent, other chemotherapeutic agent, or combinations thereof.

34. The pharmaceutical composition of claim 33, wherein the at least one agent known to treat a cancer is a hormone therapy agent is selected from one or more of the group consisting of leuprolide, tamoxifen, raloxifene, megestrol, fulvestrant, triptorelin, medroxyprogesterone, letrozole, anastrozole, exemestane, bicalutamide, goserelin, histrelin, fluoxymesterone, estramustine, flutamide, toremifene, degarelix, nilutamide, abarelix, and testolactone, or a pharmaceutically acceptable salt thereof.

35. The pharmaceutical composition of claim 33, wherein the at least one agent known to treat a cancer is a antineoplastic antibiotic agent is selected from one or more of the group consisting of doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

36. The pharmaceutical composition of claim 33, wherein the at least one agent known to treat a cancer is an antimetabolite agent is selected from one or more of the group consisting of gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

37. The pharmaceutical composition of claim 33, wherein the at least one agent known to treat a cancer is an alkylating agent is selected from one or more of the group consisting of carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt.

38. The pharmaceutical composition of claim 33, wherein the at least one agent known to treat a cancer is a mitotic inhibitor agent is selected from one or more of the group consisting of irinotecan, topotecan, rubitecan, cabazitaxel, docetaxel, paclitaxel, etopside, vincristine, ixabepilone, vinorelbine, vinblastine, and teniposide, or a pharmaceutically acceptable salt.

39. The pharmaceutical composition of claim 33, wherein the at least one agent known to treat a cancer is a mTor inhibitor agent is selected from one or more of the group consisting of everolimus, siroliumus, and temsirolimus, or a pharmaceutically acceptable salt thereof.

40. The pharmaceutical composition of claim 33, wherein the at least one agent known to treat a cancer is selected from uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.

41. A method for the treatment of a cancer associated with an AVIL dysfunction in a mammal comprising the step of administering to the mammal a therapeutically effective amount of at least one compound of claim 1.

42. The method of claim 41, wherein the cancer is selected from the cancer is selected from brain cancer and cancerous tumors such as glioblastomas, rhabdosarcomas, gliomas, lung cancer, bladder cancer including bladder urothelial carcinoma, and renal cancer including kidney clear cell carcinoma.
